SEQUENCE LISTING (29 sequences)

SEQ ID NO: 1 - DNA, 106 bp, Homo sapiens
ctcgtcctcctaccgcaggatgttcggcggcccgggcaccgcgagccggccgagctccagccggagctacgtgactacgtccacccgcacctacagcctgggcagc

SEQ ID NO: 2 - DNA, 23 bp, Homo sapiens
  misc_feature (1)...(23): n = A,T,C or G
gttgtttaggttgtaggtgnggg

SEQ ID NO: 3 - DNA, 25 bp, Homo sapiens
  misc_feature (1)...(25): n = A,T,C or G
ctcntcctcctaccncaaaatattc

SEQ ID NO: 4 - DNA, 33 bp, Homo sapiens
cgccaccctccgcagccatgtccaccaggtccg

SEQ ID NO: 5 - DNA, 35 bp, Homo sapiens
ggccatcgccaccctccgcagccatgtccaccagg

SEQ ID NO: 6 - DNA, 21 bp, Homo sapiens
tggcacatatacaccatggaa

SEQ ID NO: 7 - DNA, 21 bp, Homo sapiens
tgatggtttccaatttcatcc

SEQ ID NO: 8 - DNA, 21 bp, Homo sapiens
tggcacatatacaccatggaa

SEQ ID NO: 9 - DNA, 24 bp, Homo sapiens
tgtccctacaaaggatatgaactc

SEQ ID NO: 10 - DNA, 20 bp, Homo sapiens
gggtggacgtagtcacgtag

SEQ ID NO: 11 - DNA, 19 bp, Homo sapiens
cctcctaccgcaggatgtt

SEQ ID NO: 12 - DNA, 18 bp, Homo sapiens
ctgtaggtgcgggtggac

SEQ ID NO: 13 - DNA, 19 bp, Homo sapiens
cctcctaccgcaggatgtt

SEQ ID NO: 14 - DNA, 19 bp, Homo sapiens
cctcctaccgcaggatgtt

SEQ ID NO: 15 - DNA, 18 bp, Homo sapiens
ctgcccaggctgtaggtg

SEQ ID NO: 16 - DNA, 45 bp, Homo sapiens
tcccgcgaaattaatacgacctcgtcctcctaccgcaaaatattc

SEQ ID NO: 17 - DNA, 23 bp, Homo sapiens
gttgtttaggttgtaggtgcggg

SEQ ID NO: 18 - DNA, 45 bp, Homo sapiens
tcccgcgaaattaatacgacctcatcctcctaccacaaaatattc

SEQ ID NO: 19 - DNA, 23 bp, Homo sapiens
gttgtttaggttgtaggtgtggg

SEQ ID NO: 20 - DNA, 20 bp, Homo sapiens
tcccgcgaaattaatacgac

SEQ ID NO: 21 - DNA, 23 bp, Homo sapiens
gttgtttaggttgtaggtgcggg

SEQ ID NO: 22 - DNA, 23 bp, Homo sapiens
gttgtttaggttgtaggtgtggg

SEQ ID NO: 23 - DNA, 20 bp, Homo sapiens
tcccgcgaaattaatacgac

SEQ ID NO: 24 - DNA, 22 bp, Homo sapiens
tcggtcggttcgcggtgttcga

SEQ ID NO: 25 - DNA, 22 bp, Homo sapiens
ttggttggtttgtggtgtttga

SEQ ID NO: 26 - DNA, 35 bp, Homo sapiens
gtgygggtggaygtagttaygtagtttyggttgga

SEQ ID NO: 27 - DNA, 94168 bp, Homo sapiens
aacagtgagagggccctcacgtatacactgatccatgggtaaatggaaccgagagagtggcattgtctctgcttttagattcttgccagtaacaccactgtaagtgagtgaaggcttaactcttttgtttttggcttgtggccttaatcagctactttaacgctcttgacagctgagctttctctttcccagctctgctgagaccctgacaacaacactgaataatgtcctaaaaaattgatagtttcatttatctgcaaagacatattttttgtgtctcactgcctcttcccactccgattacattattatttctacactgcaaagctttacaacattttcagctattttctaaccatagttttcctcctaacacggcagcttcattcctgggttttctactttgattcatctcttgctcagctagattaagtgatgcagtcatctgttccagaagggctcatggatgttatattgtaaatttcattctagaaaaatttttatctatttttattaagagtttttgttccactggagtcattctcctcttcagaggcattaatgatatttatcacggatcactctgtcttcggtagctatcatttactctacagtatgtaactggagaaagtgaagaaagagaacagagattatttttgaagcaataatggtcaaaccccccaaatttgatgaaaattataaataagaaaagcaccacaaggtccaagcactacaaataaagaaaaacatacagagcacatcaaaataaaacttttcaaaacacgtgataaagataaaatcttaaatgtagacaaagaaagacacaatatattaagaggaatgaaagcagaaattattacagaccttaagtcagaaattaagaaacccagataacactggaaagtattaaaagaaaacaaacagatttttacaaaaatatctttcaaaatgaatgcaatcaaagattcagtgggccaggcacagtggctcatgcctgtaatcccagcactttgagaggctgatatgggaggaccagttgagcccaagagtttgagaccagcctgggcaacatagtgagaccctgcctctccaaaaaagaaaaaaaaatgagtaaaaattatatttcacagtgaagacaaaataaaacacttttttactcaaaagcaccaaggtaatttgtccctttccacatataaccaacaagagacactaaagaaggtttttcaggaagaagaaaaattatatcacatgcaaactttgatctaaaacacaaggataaaaagtgctgaaaattgtaggtatatagataaatataaaagactttgtgctttttaaaagttctttaatagatatttgactatttaatccaaaaaacagtaacaatgtatgttggtgcctataacattcgtagcagtaaagtgcaagacaaaatatcaaaatagaccagtaggggaaattggaggaatactgttaaaatttaaacataaagtggtataatgttatttgaaggcgggcttccacaagtaaaagatgcatcttgtaaaccctagagaatccaacaggaaaataaaacaaagatagatatagatatttagatatttaaaaataaatttcagtttacattgaataaatattatgactaattgtctttaaaaagacattagtgaagaataatgaattttttaaaaatccaaataaaaatagtgacaatgtaaaatggtaaaaacacttcaaaaactttgaaaatttggtattttctcataaagtttaataaatgtaggtatttatccacaataaatgaaaactaagatccacacagatttgtacatatatgttcattgcaactttacttgtaatggtaaaaaactagaaactacttaaatgtctataataatgtgaatacatacacatattgtagtatatgatattctactgtacaaaatatcactcaacaataaaaaagagcaaattattgatatacaacaaaacatgatgaattccaaaaccatcaacactaaacaaaagcagttccactcaaaagaactacactcttatgattttatttgtgatatactaggaagggaaaacttaatccataatgaaaagaaagcagatccattgtcgcctgggttggggttggagactgacagaaatgtggcactaagaacaatttagcatgatggaagtgttctaagtcttgattgtggtggtagtagttacacagttgatgattgagtcgaaactcaaactgcccacttaaaattacgtacaataaactacatatttattgcatgtaaattatgagaaatttcatgagaaatttgtttgtaaaatttttcatgataaattaggagaaacaacacaatttcttttttttttttttttttttgagacaaagtcgaccaggctggagtgcagtggcgcgatctcagctcactgcaacctccgcctcctgggttcaagcgattctcctgcctcagcctcccaagtagctgggactacaggcatgtgccaacacacctggctaatttttgtatttttagtagagaccaggttttgctatgtcagccaggctggtctcgaactcataacttcgtgatccacctgtctcggcctcccaaagtgctggcattacaggcgtcagccaccgcgcccagccaatttcacattatttaaaaaataactcaacataaatcagtgtttcaatacttgtgctgaagtcaccacaggtgaaaaaggttaaccttgacaggtttgctaaaataagaagcattcagtaaaagccaatgattttctcccttgttcttactgcatgctatatatattgcataagtatttaataaaagtaaatactttttcgaagttcaatatcaccacagaaccaagcatttagaagtcaagcatatccttacatttcaattatttctctgaaagaccaaaatattgatctccctgtctatagctggacttttttttaaaagcatgctatcttctgataggatataaaatgttaatctccttctcctgaactcttaaatttatatatgctttatcttccaattaagacaacggaaaccagtattaggcagtgtttttcaatcacatttcccttgctacatacttattaaaacaatgacaattactcacttcaatcgtcttggcaagtaactgtgtgtgaggaaaattatacttgtatacttcattagcgacagtgttgacatcaatggcagccaccacttgtgcaggtatacagctttctgtaatgataaatggagtatcttgaaaagaaaagtccttaagttcattatagaaagaaatcaaaatagcagaatttagtaatccatttccttatttcttcatttattagcctcatctcagactgtcagtggaaggtcaggccaggggaagccaggcatgaagtgaaatatttgaacatggacttctggttcatctctctaaacagcattccaatactaaagagaagatttaaatattttagtgcttacctcttatatttgctttcctccctcattaagagacacacttataagtcaaatagaaagttttcctaagttaaaacaaactaaaaattgaaaagtgagatatattctaaaagccctatgatttagaactttatataaataaactaccatttgttctgcaccttctcacattaatacaatgttgcttcacagtaccattgaaaatgctttaattgtttttttagtaacagctttattgagatgtaattcacatttgacaaaattcacccaattagagcttacataagtttttaaatgagatgaacaattaattggtatccaaatgtaaatggttcatttaccaacattttaaatgttgacagaccagaagagaatataagaagaataagggaaataattataaaggaatgccttcactgcccttctgtcttgctggtgaagaaaaccattttcaaaatgttttcagatccagagaattctagcatattctttaatgttagccgaaattggaactccacaattgtagccgtagctatattttacaatgaccaccctctttccccaaatatgtataaaaccaattatgtgcatgccctgacccaagtcaggagaaagtgtgaagttttaaaaatgaaagtatttgagacttcagaaaatcatcagagaaagcaatacaatattacatagtagatccaagtagccgattctttaataccattagttgattaatagttagctgattagcttaccccaaatatcaaaaaaggaaaaaaaaaaaaaaagagtggtgtgaggaactaggactcatgcagcaggcacccggaaataaaagcagaccacaggagacagggcaaggagaaaggctcacacacgctcagaaaacaatcataaccttccactgaggcctagaagagatgaggccaggaagaaaagaaaggcacaaatgtctccaaggattgatactaaatggctgctaggctcaagacactcagcaggtatcctgctgaaaagtaagatcccctgcgtgctgcaaacttggtggtctaatcagtaatgccatagacagctgcctcttctgcattgaagtgcaataactagttactggccttagtataaaataaatttgaggttataaattgtatctgttaaagccatcagatcttgaaacataaatcaagaaggcttctaaacaaaagaaaatattaatatataaggatccatttgcaaacacaaaactatcataattcaagtcaagaattaagctactactaatcactgtaagagagctgaaaattaaataaacttcattgtactattttactgttttgttaaatatgttgtaatcagcatgatgtagcctgcttgtatttccaccatatatgaagcaaactgggattgcctgacattggttaataggacttgtatcttttttcaggcagagtgaaaatgtacagtgatgtgctgaatctgcactgtaatcaataattaattttatgttcttgtgttatatgcctggaggtcataaaaactgacaaaaatcaactgccaaatgagtatttcatatggtgatccaaaaactgatgaactgaaatttctttctcaggaaagaacagattttcaaagccaataagcaaatgcactgtttgcatggattgatgcacatattttagactgttataaaattaggacctactgcataacttaaaaagttatttctattaagagaaatggatgtactaagatattactgacactaattcaacttgccttctcaaaatagaattggggggtggttattgtttctaaggaaaaggttaaacttacagataagttttaaaaagcaaacctgtcaattctgtcaaaatcttaacttgaattccaaagactagttgccttgatatttcttagtatacagtttaaatctacaatctccaaaattctttacttttaagttacatatatagattttgaaaatgtataattaaaaatgtcaaagtgaatataattctatagaattttgcctatgtgagctccatcacttgagtgatccaagagtagtaattctcaaaaaggaggcagaatttgggaaagggattcgtgagtatcttttcattgaaacagaggaagaggatgtgttttcagtactttgatgtattactttgcctagactttattcatgatatttcagttattattcaggatttcatatttattaacaggaagcctggtttaaaaaaaaaaggtgagatatcctacttagagaaacacatttattttatttatttatttatttatttatttattttttgagatagtgtctcactctgttgcccaggctggagttcagtggcatgatctcggctcactgcaacctccacctctcaggttcaagcaatttgattctcctgcctcagtctccagagtaacagaaactacagacacttgccaccacacccagctaatttctatatttttagtagggacagggtttcaccatattggccaggctggtctcaaactcctaacctcaagtgatctgcctgcctcagccttccaaagtgctgggattacaggtgtgggccaccacgcttggctgaaaaacacagtacaaattattttaaaaggttgtcagattggctccgaaaggaatacataaggacatcacgcttccatttatatgaggactaaatttaaaaggtagatttagttatttcaagagagacttctaggtaagatgttagaaatgtcactgaggatacatacataattcttgctttcaatatgcttcttacaaaaatagacaagaaacacacacacacacacacacacacacacacacagacatttgaatagcaatactttcctttctttttctgatgattcttctttctggaaacgctgtagggaagaactttttgttggtagtagtgaaaagctttacaagttcacactcttcttgactttcctaaccaaataaataaaaaaatttaactgttcttgtgtagttattaatgtgggaaaaaaatcaatatttacaagatgtaaatgcaaatgtaaaaaatgtaaatgtaaagacgttatgttagataatataatgcagtttgaagttttgaggaggaccttgagaaagaaaattaaggcataaccagtccactttgaaataaggaaagaaatttttaaaattctattccttctttattatttattttctaatctatatctcatataggtgagtaactactgaattactcattatactcacacctgagacatttaatcaagtatgttaacactgacatctagttgtaagatgaaagatcagttgaatacatccaatgtcctttcattcaaagtgctggcttagttaggaataaaagtactaaagaacgtacagacccataaggatctgaacttaaaattgctgccaaatgttttatcactattagtaaagtatattgtttggttctttttaaaagttcttgtattttgctgggtgaggtggctcacacctgtaattccagcactttgggaagccaaggagggcacaccatttgaggttcggagtttgagaccagcctggccaaaatagtgaaacctcatctctactaaaaatacaaaaattagccaggcatggtggcacacgcctgtaatcccagctactcaggaggctgaagcaggagaatcgcttcaaccctggaggcagcgggtacagtgagctgagattgcaccattgcactccagcctagatgacagaatgagactctgtctcggaaaaaaaaaaaaaaaaaaagttcttgcattttaaaaattgctgataaattatttccaagaataaaataaaaaatacattaaaaatatttttcaaagatccagtttccttagaaagtttccatagtcagctaactgtccaaacaaaatttactttgtgtcaagacataccttagattattattgttactaacaccagactaaatataagagaccaccagccaaaatacggataggaactgctggagatctatgaaattatcacgtccttgacactatatcttaggagaatttatttttcaacaaaataccaaaagacataccagagccagagaagtggtggaagagagaaatatggcaaattaatgtaaaaaaaaaggagcacagtgaaggaatctacaaggaatggaaaataaagatgttatgtcaggtagtataatgcaatttggagttttgaggaggaccttgagaaagaaaattaaggcatgagtaatctttccggaacgtctctagaaagtctatttaaagtcatgttgatgtcatgaggtagctaatacagcttctccagcccctcaacccacaacctcgcaccagccctgaagctttccttggcagaagagaagagaaaaagcaatttagctattcactttcagcaggggctacagaaaggccacatagcccagcattacccttagtcctgaggatgcagggacctcaatttccaagggcataaactggtctctaaaaagaatataggtaacgaacgcctaggatacagtaaatgctcaataattattaaaattcatattcactgcctggcatttatctggaatattatagattaaatagataaaataataatgataatgattattttaactaaaatttaaaaatcaaaattaaaacaatcattatcattataattaaggttagaaaattagaccagtgacaaaagcttaagactcatactgttcatctgggagatgaattgcaggagaaatatttttgaaagagtttcaagtaatgtaagaagagatcttcacaaatgacaaaaactagctgattccaatctctactaataaaaaaaaaaaaaaaaaggaaaaacagaaacgggctaaaatttaatcccaagagactagcattgatgggaaaagctttcctgaccttgctatcaaaccctagaatgaaagagagaaatcggaggtattggggaaaggatttttttaacacagggcagaaaaattttcttcccaggatatttattacttgttttctccaatttggaggaggatagtcccactaagcaatccctaaagacactttctagccctgatactctatgagcatttaagtaagaagtataggtcttcagatcatcttttactacctgccattattgacaaagatcttggccttttcaccctaattcactggttagaaactaaagaactgatacaagagaaatgctcttttatggctatgtttgtctattaatttgcctttcctagatactacgtttaaaataactattaaagaataaaaatagtaagtgaactacgacaaacaatataaattttctctttctgccgggtgtggtggctcatgcctttaatcctagcactttgggaggctgaggcgggtggatcacctgaggtcaggagtttgagaccagcctggccaacatggcaaaaccccgtctctactaaaaatacaaaaatcagctaggcatggtggtaggcacctgtaatcccagctactcagggcctgaggcagaagaatcgcttgaacccacaaggtggaggttgcagtgagccgagattgcgccacttcactccagcctgggcaaaagagcggaactctgtctcaaaaaaaaaaaaaaaaaactctctttccatattcggtacagagaaaacctgatttgtgggaatcaacatgaagtaacattgcattaatgcttaattttttccttctacttataactgtctattaggagagaaactagtatttcctagacaaagctgcaccttaggtgaaatagtgccgtgctttttttcatgataatcaatctttaattcccttgtccctcttcccatttcaaactacctagcaaaatgctaaactgaacactaagcaaatgagtacttaagaaacagtatgaaagatggatagcaaacaacatatttttcctatttcagaactcaaggaacttcacaatttctccttagtgctcagtctctttgaagtaaattttacatcaaattaaattttgctaatgaaattttactaatatgttttactaatgactcaaaaatatgatttctagttatactatctctacaaaaattatttgcatatcccaacatttatttcttcctcctcttcaataaccaaacatcaaatttattcactgtggcaatgcaccctgctaaaaggatatctgccacccctctacagctagatgcagccatggactaaagtcagactaatgagacagaataagtgcatcacactctcagagagtgttctataaaggaagagagtggtagttccctatgctctctggcctttccactggaggtttggaaagcagatgtaatgacttgagctccgtttgctgtgacggaccatgaaggctgatccgtgcactctcatcacactccagttcactctgctgtcacatttttcccaggacagtggctccctcactagactactcaatcatctcagtgctgacaccatgtctcattcatatgggtactctcagcacccaggcattgcagtaattggcacagaataggcgctcaataaacgtctattaattgaatacatacaagagaataagtgaattatacttcctaaaatcaactccagattattactgattaccctccttgttctcctgtttctgagaacactgctagcttaaaaagtatttaatatgtctgaacttatatgattatctatctcaatgctcatttttaagtaattgtttgacatttaggtcaaattttttaacacctacaatgaagttcccaggaaacctacaaaattctatttatgttacaaatgtcttaaatatagtcccaatagtgctattgtcaggcagtaagtaaatatctgtgagattacctatcatgtactgagcactctactgaggatacaaaatgagtggacatttgttttctgttttaccagcatctggcccctccttttctggcaatagtcccagattttctttgggggaaacttacctcactcatgctctgttgaagtggttcagatggcctcaaatccccttctcctagcttctagaagcctcggtcaagtgcctgagccagtaagactagccagaccggtttcaggatgggcatgcaactcaaaataagccaattaggcacaataagaggtgacccctggctttttcttgtgtaactattaatagcaagagtagcttgatctttcttaccacgttaaaccttggatggagataagttcagaggctactacaggcattttgatgcaataaggggagaacatatataaaaatgcacccaacctagaagaaacaaggctgagatacatggagaaaaaaactggtttctggtgacatgatttgagcccctgcttcaaactctacccctaactgggcctacatttggacctggtcaattaaatgagtccagagttacaacttttaggaaaccagtctgagctaagtttctgtcacttgcaaataacacttctaagtggcaatcaggtgaacacaaactaatatgccatttgttcctatgaggagaagcatttcaaattccaaactggcaaacagagcaatatgaatgcaaaagtcctgtaaataagtaattaattaaccatacatctacaaaatagaagtattcttcttgataaaactgttctaaattgaagaaagaaggtcaagagtcttcaatttggggagtgaagacttccttatggtaatacccaatcacttatttctatatatggatactaaatacaacaaatacagagagagagctacttataaactatagctcccatgtcttttaaggagcacagaatattttcagttgcccagtctcatcaattcttacaaagctcaagtcctgaaagtaaaacagttaaaatagaaatttcattacaagcaattcaaaaataccaaattttaaatgatcgataaacatataaaagatgccaaacttcaccaataatgacagaaatccacattaaccagttttacctagcagcttggcaaagattggataaatgaacactgtctaatgccagcaaggatttaagaaaatgtgctgtccttcatgtcatttgtggtataaattgacataattcttctgattattagacctagttatactacatctggaaatgttcgctaaggaaaattgtaaacaagtatgtaaagataagagtgtatcattacatacatgtatgaaaatagagatgttaaaaagataacatccatatttactgtcacaaagcaatatccatattatttactacatagagcagttacagaatatcttgagtaaaaatccaagttttataaagtgtatgtgtatgtgtatgtgtgcaccttaagacagagaaaacagccaatatcaacagaggctatcccctggtggtgagatttggaggtaattcttctttctttaaactcttttgaataattcgatttttttctcattgagaatatactattattttatttatatctttagttacttttttgagatggactcttgctctgtcacccaggctggagtgccgtggcatgatctcagctcactgcaatctccacctcctgggttcaagtgattcttctgcttcagcttcctgagtagctgagactacaggtgtgtgccaccacgcccggctaatttttgtatttttagaaagagacagggtttcaccacattggccaggctggtctcgaactcctgacctcatgatccacctgcctcggcctcccaaagtgctgggattacaggcatgagccaccgcacctggcaagaatgtactatttttataattggcaaaaagcaaacaaaaaaatgactcttattttggaacgaaagaattcaatgctgctcttggttttgaccctgagaaacaacaaaaggaaagtaacaaggtcagacaagaaaggtttggctcttttctcaaggcatgccaattgtgctgtgacctgtcacaggcacatacacaccagtaaacaattataactcattataattacaacaattgtaactcagtgcattattttacctgctaactcaacaataatcaaaagaaaaactaaaaattaaactaccttatgaaagtcttctaaagatcatcaagtagctagcatacgcccaaaaaatgtagagctgaaagtaacctattcacatgcttatagaagtcaaaagatataaactacttaaaaagtcaagaaataatagaaacacatgtgatgcagtcaattacaggtgtgaacaacaaagactttgattttgttttacatacatttgttttgagtgaatgaccactcatgaatggttccttactataaaatgtttacggtcctctttactgatgctttctcataacttatgaggtgatcatttctgacatatttatggaaaagtcaatgaaaaaatgagaagtgacataaacattagtactctatatctagttaactgaattttttaaagacttgtaaaaatataattattttaatattttataacatatagtataatatatataataattaactcacaaagatccctaaaattagcctaagaaaatcctctctaaatttgtcttcccattctgagcctcctttgctttatagttagccctccttgaaagcttctaattttcatcattattgactcaataaaatttcaaatcctatcctgcaaaacactagcagtataatttctgctacagatacctctaaaaggagagttaagttttacaattttgcactaggccagaaagtataggcttgtcataagacctatgcacttcaactgcctcactcatttcccctttccaagtctcctcctaactatatcattctctacaaatctttttccactaagccacacagtattcagcattaggcattcttattttattttttatcaaagctatcttagggacagtcttatggaattttttattttgtgtgtgtgttcatacgtcctgatgaagatttagaatatggatatctgtttgtgtatttctttctctctttctctgctagaattctgcatttaaatcctagcttggctatttccagttgcgtgatttaggcaagtttctttgtgtctttaagcctcagtatcttcacctattaagaggataataaagtacccgcaccatagaattttctgaggattaaatgagttaataactataataggacatggacatctttggggattactatttgattagtgtctggcatataataaatgtttgccattattattattgaaagtgcaaagactttttaaaaatgagaatatcaaatgctagaaaggaccaggtgtgatttaaaaatcgtcagtgcggcacttctcaaaagaagacatttatgcagccaaaagacacatgaaaaaatgctcatcatcactggccatcagagagatgcaaattaaaaccacaatgagataccatctcacaccagttagaatggcgatcattaaaaagtcaggaaacaacaggtgctggagaggatgtggagaaataggaacacttttacactgttggtgggactgtaaactagttcaaccattgtgggagttggtgtggcgattcctcagggatctagaactagaaataccatcccattactgggcatatacccaaaggattataaatcatgctgccataaagacacatgcacacgtatgtttattgcggcactattcacaatagcaaagacttggaaccaacccaaatgtccatcaatgatagactggattaagaaaatgtggcacatatacaccacggaatactatgcagccataaaaaggatgagttcatgtcctttgtagggacatggatgaagctggaaaccatcaatctcagcaaactattgcaaggacagaaaaccaaacaccacacattctcactcataggtgggaattgaacaatgagaacacttggacacagggtgggaacatcacacaccggggcctgttgtggggtgcggggaggaaggagggatagcattaggagatatacctaatgtaaataacaagttaacaggtgcagcacaccaacggcacatgtatacctatgtaacaaacctgcatgttgtgcacatgtaccctagaacttacagtataataaataaataaataaataaataaataaataaataaatatggtcaatgcagttgtgagcatcaattaacagtcaatatttcaatgaatatttggtggtatacatttaaaacccaaaaaattactttggattttttagttttatgttttggcacatttcttaaggtgtgataggcaaaataataatccccaaagatatccacatcttaatccctgaaacctgtgaaaatgttaccttacatgggaatcaggattttgaagatgtgattaaacttgagatgaggagattatcctgaattatgcacgtgggcccagtataatcacaggaaggactgaggaaggttaaaatcagagaaggggatttggagatggagtagaggtcagagagaaggctttgggatgctacacttctggcttcaaagatagaaaaagggataatgagccacgtactatgggcagcccctagaagccagaaaagccaagcaaatgaattctcccctagaacctccagaaggaatgcagccctgttgataacttgattttaagacttctgacctccagaattataagataataaacctgtgttgctttaagctactaagtttatgtttgttacaacagcaataggaaactcatacatagataataattagagatgtaaagaaaatatttatatataggataagtgatgctattttatttataatagtgcaaatttatgaatgatctgaatgtcccagaatagatgattaattcgaacaaatactgtactttcataaagtggagtatgatacaaccattaaaagtttataaattctacatttaatggcaagcgaaaatattcaagatattctaagtaaagaaagaaagctataatttattttaatgttcatggtcacattagaataaagattttaatctaagccttcaccagtgtttatgtcttcaccagtgttatgtcagtgtttatgcattagcatcattggtgagttttttaaattttcttctctatttgtgtagcattggtattgcccagtagagcaaaatctctggaaaaaggaacatgactgtctcgttcaatgctgtatcctcaccatacaacacagtaagcaacaaagaaaaatcactccatagttatgtactgaataaatgaatattttcagtgtgtcccaattttttaacaaatgtattactaatattgctaaaaataaaaaacaagaaatgcacataataataatttttaaatattcaacttcactagtaatcaaaaattaagataacaatttcacctagtaaattatatgggttaacatattatattggcatatttatggtcacagagacgttactgaatataaactagtatgtccttttaaaaaaataatttgacaatcatcaacaacctttaaaatgttcatgtactatagcttattcattaattctacaaatatttgttgagcatccaacatacaatggtgcttttagatccaaggatacagtggtgaacaaaatagaacaaaatgcaacaaaaaaatcccaccctcaaggagcttacattctagaagaggaggcagtcaattttaaagataaacaagtacaatacaaaatatgataggcagtaataagtgttaaagtgaaaaacaaaacaggccagaggatagggaggcagggcagaggatacaagttttggtactgagaacagttcactgtgcatctttcctgacctttttctgtgtctaaacacaaatacattacacacatacccactgtatgtgcccaaacataagacaactagaataaacgttaatcccctattttccccatgaaaatatccaaaattagatttctggccattttcattatataaatgtttaaggatgtagccaaaatagtcaaatgacttcatattgtataatttataatttaaacatactttattttttatattcagtttaattactatttttacattaaaattaagttctacattagtaaacttaattttactaatgttttacattagtaaaacaaactctgttttttgaacttttttttaactggcatgaagaattacagctcttaggtgttaacatgggaagggaaaagggtatttatcccatttaacaggtctggctagcttgtattcaataaatatgtgcttgaagaagccatctgaggtaatctttaggatctgttatgaatttataaagatataattccaggtaatatctttaaaaaatagagaaaggatttatcccctgactactgaacgggacaatgcttatgaaaaccttttaccaaccctgtcaagtgtttattcatgctctgcaaaatattttcagcaagtatttcctgtgagtttataaccaggttcatttatgaattcaagacatatttattgagcatgactgacgctggcgctggctctggctccaggcagagatgataaaacaacgaatccctgctcccacagagcttcacacaaggagaagcctccaacatattttatacagcactatcttagcatgttcatttggaaatattgtatattttctactcaaagttcatgaaatgattttattctactttctcatgtgcctctttgaaatcagggtcactatcatcaacagagccaccttttaaatcatctaacagtgttcaaggacacaccaactttccttcagtcagagttacttgagaactagccctttttgagttcgtgtataatgatgtcactggaaattttatgcccaccacaaatacccagtagctatttgtgatttccgcgaagtaatcacctactgtattgctttataaagtaattttcaaaaggcttgtttacaataacatccaacacttgtaattgtgaggtcatcctcctaaaaattacttctagatctttaaattcatttctatggtttctggccaattccattgagtgacctcaataagcatctagaaatgatattaagtttcttcaggctaatgtagtttatccaagcagtactttattgatttgctatttttgaaaagtttagtaattgagtaatatgaagagatttcctttcagaagactaaattataaggccatactctctttctgaagtataatttttcaggtaaaatgaatatctcatatagaagaatttatgccaaacatgtctataagagtgtatgtatatatatttgtattttgtttttgttcttagttaaaatactgggatcacaagcgttttgaatttacagtcatgaaggaaatgcaaaagatgatgtcttcatagcgaaaaacccttggattttaaatatagtgcctttgccatatatggatatgcaaagccataaaaaatttagaaaaatggtagattcacagctttttcttcaaaccatggtaccaacattctgacagttcaaaattaatgaaaagaaatctcactggatggagaacaagctagagattaagtcacaatcaacaaagaacatcatttaatctgaatattcaaataatatagttttaacaattatctcagcaaacaataacacaaaaataaattgagaacacaagaaaaacatccattctgaagaagcagagaaattattctggtactttattaccatctaaaccttagcgatctattaaagtttagcatagttttaaaaattctagttaatttaaaatcatatgctaacatatgatttcaatttaactagaatttttaaactacaaatcctttgaattataggttctttttgaaccaaaattcaactctaatgaaggcatctgccaaagtctatggggtcctgtttgtgctaaaagactctggcatttgtttcttgagtctcattcattgagtactagagggtaattaacttcattattgagtttgaacaaaaatcagtaatggatgcacaagaactatttaatattaatgttcagctctctaatagagctgttttactttacacaaaggggtttggctaggtttgtgttgatttaaagtctcagaatgaaaataattagtggatataagtaaaagaaaccattggtccaggcgaagtaacggtaatttttaaaattccaacatacactttacctagtcgctaaaaatgagatataattttacgcttttagacaagaggagagctggttggcactattcctgggtagcacttttcaccaatatttctgataatgttccatgggaaagcaaaaggcaacagagattctaaaaaggttccagacaccaaactgtacatgtaaccttgaccttatttatcattatgataataatcaccgtgtaattatgtgtagatttgctctcatacatcaacttcttttcaactttgaaaaataaagagttcatatttcttgacaggaataaaagagcaaaaaaaaaaaaaaaccacttaaacttaattattttggccatagctcactgttagcatatgatttaaattttacagtgagtaaagaccaatttattctcaaaagccctgattattttgacaatatttaaaatgttttaaataaaaagaacaaaaatgttttatcatgtattacagaaaaatatttgttatctattaaaatctcacacacaaccatgcctacaaaaactcttcatgattcagacttaatacttaagtttaaacttatcacagaaggatgatatctacatcaagctgcagatatttcacagcaagcatgcctatagttcattttttcatttgttcattcacgcattcaacaagtatataaaaggattattccagacaaagcctatcctatcatggagcctacattctagaggagagagacagacaataaacaaacaagtttaaattatgcctaacaataaaaagtactatggagaaaactaaaaaccagagcaagacagcatgggagaagggagtggttctagttttatgtaggtcggttacagaatgtctctttagcagcaacctggaagagatgaggggatgggcctgacagatatcagggagaagcactttctaggacagagagaagagacacagcaaaggcccagagatgggaacatgctggttttgtaggaagaacaaccaggaggccagggagcctggagtagagtgagcaaggggacagccacaggagatgtggccccagagagaaagtgtctctagcccaagcaggcctggggacccattcaggactctggtttctgagtggtcggagaagctcctcaggagttttaatcacagtaataacttatcagatatattttttaaagatcactctggccactctactgaaaacaaacttgcaatgccagagcagaagcaagatcagtaaagaggctactgcaataatgctttgtccagggtcccagaaaagtagctcaggccaagcttagctgcagatgctttatctggaaggaaaactccgaagcggcaagagttagggcagtgaggcagagaaggagggagtgcattagcacgtcggtcaatgcttcccagaaacccagccaactgctctggcaatgagggatttggtagagccaggctggatgggatgaccacacctcagagctgtcaggggagggctgtatctgctgtgggagatgatgagatgccctgcagtagagacgctactgggtctgaaatagcacttcccatcttctcactggcatttgagcaaggagcaccattgcgggaggagggagagagtgtatcttctggctcctttctcatcctgtctgttattggtcaaaagtccccccaaagcgtgactccccacctccctgctacctacacacacaagtcaggttttgtgatctgactcctcaagacagctactgagcagacagagtgcatgcccacagcctgacacttcacctgagtccaggtacaggggtcgacaatcattctgagggtcaagctcggccatcaccccctgacagtgtcagatgaggcaaggtggcattagtcagggtgtgacactgaaccagcagccccaggccaggaggcaggtggggccaagcaaatccgggcagcgcaaaaattaagtctgacaagacaggagagagattatagatcaggtggtaaaggtgaaaatggagagaaatgatagagttataaatatagtctgaaggtaaagcctgtagaactggctgatgtgggtgaaaaagaagaaacaagatgactccaaggctcaggtgtgggcaactagaagaacaaagccacctgttattggggtgaggcaggttacaagagtggcaagttttggaagggatcagagaccagtagttcagctttgggcattctaggagaaaacagagatagtaagaatgacaatacctgcctaatagaaatattacagagaagcttatttcaaagttcgtccaaattatgaacatagttggctaatatgtatagccctggcgcatcttttctttcacaaccccagcatctccctcagctccatcactgcccttttgttgcccatcagacatttccggaaacacgcactaaggagcactgttttcacaatgtaggtgtgctctccttccccgcaaaacaaaagggtgggcgaggggcaggagcagcagggtccttgcccaaatataaatacatatgggaagcactgttttaagccaaacagcttctttacttctcaaaacctgatacatttcacgtgtcttgagaaggtctcaaggaaagatgttgtgtggagagtttctaaacgtatttgaacatcaaggtacactgaataaacaaaatccatatgatcctctcaaaagatgtggaaaaagctttcaataaaatccaacatttcctcatgataaaaacccctcaagaaagtaggcatcaaaggaacatacctcaaaataataagagcatctatgacaagcccacagccaacatcatactgagtgggcaaatgatggaagcattccccttgcgaactgaaacaagacaaggatgcccattctcaccactctattcaacatagtattggaagtactagccagagcaatccctcaacagaaagaaataaaggagccaggcacagtggctcacacctgtaatcccagcactttgggaggttggggtgggctgatcacctgaggtcaggagttcgagaccagcctggccaacatggtgaaaccccgtctctactaaaaatacaaaattagccaggcatggtggcgcatgcctgtagtcccagctacccaggaggctgaggcaggagaatcgctggaacccaggaggcagaggttgcagtgagccaagattaagccactgcactccaccctgggtgacagagtgagaagacgtcaaacgatctctctttgcagatgatattattttatacctagaatactctaaagtctccaccaaaagtctactggaactaataaatgacttcatcaaagcttcaggatactaaattaatgtgcaaaaattagaagcatttctatacacaaataatgttcaaactgagagtcaaatcaagaatgtgatcttctttataacagccacaaaaaacatataaaaatacctaggaatacatctaaccaaggaggtgaaagatctctgcaaggagaatgacaaaatactgctaagagacatcatacaggtgacaaacaaatggaaaaacatcccatgcttatggattcaaaaaatcaatgttgttaaaatggtagtactgcctgaagcaatctacagattcagtgctattcctgtcaaactactagcatcattttttcataaaattggaacaaactattctaaaactcatatggaaccatataaaagcctgaatagccaaagcaatcctaagcaaaaagaacaaggccagaggtatcacattaatggacttcaaactatactataagactacggtaaccaagacagcatggtactggtacaaaacagacatatcagacaaacgaaacagaatagataacccagaaataaaggcacacacctacagtcatctgatcttcaacaaagtagacaaaaaaaatggggaaaggactcactatttaataaacggtgctaggatagctggctagccatatgcaggagaatgaaactagacccctacctttcaccatatacaaaaattaactcaagatggattaaagacttgaacgtaagacctcaaacaataagaattctagaagaaaacctaggaaacaccactctggacattggccttggaaaagaatttatgactaagtcttcataaacgattgcaacaaaaacaaaaattgacaagtgggacctaattaaactacagaccttcggcacagctaaagaaactatcaacagagtaaacagacaactcacagaatgggagaaaatatttgcaaactatgcatctgggaaaggtctagtatccagaatctaaaagaaacttaaacaactgaacaagcaaaaaacaaacaaccccacttaaaaactaggcaaaggacatgaacagatacttctcaaaagaagtggcccatcaacaaaatgaaaaaaaatagtcatcatcactaatcttcaagaaatgcaaatcaaaaccacaataggacagcatctcaaaccagtcacaatggctacgattaaaaagtcaataaaaagctaggcgtgatggctcatgcctgtaatcccagcactttgggagaacgaggcaggtggattgcttgggatcaggagttccagaccagcctggtcaacatggtgaaaccctgtctctactaaaaatacaaaaattagccaagcgtggtcgtgggcacctgtaatcacagctactagggaggctcaggcaggagaatagcttgaacccgggaggcagaggttgcagtgagccaagattgcaccactgcactccagcctgtgcaacaaagcaagaatccgactcaaaaaaaaaaaaaagtcaaaaaaaaaaaaaaacgcagatgctggcaaggctgcggagaaaagggaacccttatgcactgttgatgggaatgtaaataaattcagccatggtggaaagcagcttggaaatttctcaaagaacttaaaacaaaactaccattcaacctcgcaagctcattactgagtatacatgcaaaagaaagcaaaatcattctaccaaaaagacacatatacttgcatgttcatcacagcactagtcacaatagcaaagacgtgcccatcaatggcggactgaattaagaaaatgtggtatatatacaccattggaatactacgcagccataaaaaagaacaaaatcatatcctttgtagcaacatggatgcatctggaggccattatccttagtgaattaacacagaaacagaaaaccaaatacctcatgttctcactaataagtgggagttaaacattggatactcatggacaaaaagatggcaacaatagacaccggggactactagagagagaaggggggcagcaaaggttaaaaaactaactgtgggtactatgctcagtgcctgggtggtgggatcattgatacctcacatctgagcatcacacaatatacctaagtaacaaacttgcacatgtaccccttgaatctagaagctgaaaaaaaaaaaatttacccctaaccccaaaaaatactttgcagtgaagtaagcacacattattcctcacaagaaaagataagtaattcaattgtctaatgggaagttaaagttctcttttactgaatactcaaaattcatcttacgtatttcttttttgtatttcaagtttattaacttccacagctgccagagaagaaatgataaatttattttaataggacaactttttacgttgcttcactttaaatatgacttgctatataagagtaactagaaggaaagaggcatgaaaaggaaaattttataatcccaaagacaggtatgtataactgaaaaaaaaattaagcggttcataggccagcccctaacaactgatgctataaaaatatccttaggtgacattcatatctacatacaaatacacttcatcgtaatgtagttgcctgattcagttacaaacacacccagttacaagctggtctagtagactcccaagttggcatgtaagtcgttgaggaacaaagacaaaagagattcagaaaatgaataattaatacgtgacaaatgagatgcttagcattttaaaagaaaatattaaggctgagtaattcttactaaacacaatgaagtattatggggtaaaactcaaagccaactccttggaagagctttagaagctatttggcagacaggcctttttgcttttaaggctgactccaatatttgactatatagaaaacagtcatccagctagagctttcctttattcaccaccttaaaccacactttactcttctcctgatactacattttaaaatgagaaaaatacagaatcttatttgcatatatttgcaggtgaccttgcaggctccactgatatataacctgtgacactacttcttccactgctttctgaaagaagatattttcacttggaagattctcattagccaaaactttgtagactgtgcagacataaatatcaaagtacacataagtagtttctgtataggtcaaatatgtatttaaaaatgtaatagacattgttatactgtggccatctagaatctttgcctatgtttcctgtgtttgaaaatttgccacagtatggatccttccttccccaaatagaagccagaactcaccttctcagcttcccttgtctggccgtggtatctagtgtctaccaaccacctgcatccacatgataagacctcagtttagagttagcagtgtgaggagagacttcaattcagaggtagcaatggaagaacttaaacaaacgaggaaacccatctttctggcaagggaggtggtaagaggcatccacctctcaagggaaaactgcgacagcacttctggcttccagaacccagcactgttggtgtaagctgtgatgtcgggataaagcttcactgtggccatacccacactatagtgtgggcagtgctcctgttcacctaacctgtttgttgggtgtcctggagattctgtgagtttccaaatgtttttaattttttttttcctgattatgttagctagagtggattctgtgggttgcaactaagaactacatataatacaaaaaaagaatctgggctaaataaaccttcatcatttgataaaacgatagcagaaggattatcaattcaatagtaacctcaatagtaagctagaaagagtgtggtactgtgctagaggctgtacaggaacagaaaggaacagcaaagaatgttacatttaagccatgtaaatacatgggaaaatagatataaataaagttaaatgaaatttagaaaatacagaagcatctatgtagtgactgtaaaaatgcaacagcatactggatatgctgataaggatgaaaaacttttattgacatattttttaaatgttcactgggtttgaggcaaatctatcatgttgctaaagaaattaaatatactttttataactgaaaattcagtaatgtggggaaaaatatttgcaaatcatatatccaataagacattaagatccagaatatataaagcctacaactcaataacaaaaagaaaattccaaaatgggcaaaggccttgaacagacacttctctgtattgaagaaaacaaatgactaacaggcacatgaaaagaccctcaacaacccggcacagtggctcacacctgtaatcccaacactttgggaggccaaggtgggtggatcacctgaggtcaggagttcgagaccagcctggccaacatggtgaaaccccatctctactaaaaatacaaaaaattagccaggcatggtggcagggacttgtaatcccagctgctcgggaggctgaggcaggaagatcgcttgaacccgggagacggaggttgcagtgaactgagatcgcgccactgcactccagcctgggcaagaagagcgaaactccatctcaaaaaaaaaaagaaagaaaaagaaaagatgctcaacatctttagttattaagaaaatgcaaatcaaaaccacaataggatatgatttcatatccatcagaatggctattatttttaaaaaatggaataacaagtggagaatatggaggtagaggaaacggagtgctcatgcaatggaaaattgtgccgctgctgcggaaaacaggctggtggttcctcaagagttacacagtattatcatatgatccagcaattccactgctaggtctataccccaaaacttaaaagtaaggactcaaacaatacttatataccaatatccaaagcagcactactcacaatggtcagcaggtaattgatacaactcaagtgtctatcaacagatgaatggatacacaaaattgtacagctatacaatggaatattattcaaccacaaaaagaactgagatattgatacaggctacaaaataaatgaactttgaagacattattctaagtgaaataagccagacacaaaaggacaaatgtattattctacttatgtgtggtacgcagagtagacaagttcataaagacaaagcagaatcaaggttaccagggactggggggaaagggaatttggagttattgtttaacaggtacagagctctgtttgagatgattaaaaagttttagaaatgaacaagggtgatggttgcacaacattgtgaatgtacttaatgccattgaactatacatttaaaaatagtttaaatgatacatttcatgttgtgttttaccacaataaaaaaattccattatgaaccttatctagaaatcagtcaaaaatgaagttcaatattattccttttgtgctgcttattatattatttaatgtatagatccaaggaagttagtagttctaaccagaacaacggcaacatagcatagttaaaaggtcaggtaggtagacctaggttaaaataccagttctaccgctaactagtttattacatacttaaaaaaggacctgaatttaaaagatccctaataaacactaaagttagctgctgttattagtctttattattatattaaaaaccacctggaaaggagcaatgcattttattacaggtaaattttaaactttagtacatcaaataagtgaacatgggaaggcaaacaagtgtggaaaaatatttgcatcaatatgattaagagtctatatccctaatatgcagatttccaccgaaaagatgagagagcagagaggtacagggaggcaggaaagttgctttggatcacagggtcagccagggaaggccaatatatgaactaagacatgaaatgtgaaccgaagctagagtgaaaaagtctggggttagggagcactctggatgaggcaacagcaaaagcaaaggccagaacatcgaagaacttcgagggtttgaggagtagagagaaagaaaaacagtaagcctcgacaagaaaggctgaggaaagaattcagggaatcgtttaattagaatttcagctttgggtgttgacggtgaagcgggatgctgaagggagagtggcagataaaattgaagagaagacagaagccagaactcacaagcctgttaagatgggactgtgattacaatctcaaaagcaaccatttaagcaacgaagtaacatgatctggtttactgtgcaaatgatcatgctgcctgctagaaggaaaacggagtagggaggcagaatggaagtaaggagagaagctgggaagctatgttaataatttaaattacagaacttaatagcttggattcacaacagtagaactggcaatagaaaaacgtgtacaaattcacattgcattggctaataggagagaaaatattttactctttcattaatcccaaaactgtcacttaaaattacacaatttcccctaacaaattagcaaggattaaaaattgataatacatatccctgatgaatatgtagttaaggcactcaaaaatctctgaaaagcaatttgctatcctctctctcaaaagccttaaaaattctgtttttcacttagtaactagcacacagttaagtcttactgttgaattaattccaacagttagtaaacatgcttaaagagaaaatcggcattatgctcaagaatgtaactgtagcacactgtacttgcaaaaaaactgaagaatgatctaaatattcgacacttaaggaatggctgaattacagcttatgataaagtactaagcaattcattaaggagtttcaagatatgggaaaacgcggacattatatcactttagagaattaacaaagagtaaatagtactagagttttgctttgatgctgctaagttttaaaatgagaaataaagatagccggcaaacaagtactaaaaacttaatgatcattaacctagtctagaattgaaactcaataaatctgctcaaaaaacttatacgctaaacccattaaatattgtctgtatcttacagttactcttgttactcttggtcctaaataaggaagcagggaagtttcagttcggtggttgtcttagtaatttaagaataaacatgcaccttcaaagcctgtttgtcattctgtatccccagctcctagcacaaaatctggcacagaataggagctcaataaataattttgagtgaatatcaccaaaagtaaaactttcagttccaactcagcatgaaagaaatcaaaaacctcaaaatcaaagacgtgcataaaagtaatagtttgcttgataatatctcgtgatgtcgttttaaagtaatagtgataataatgtataatatttggagtggttacaatgtatcaggcactaacaagcacttgacagactttaatccttataataatcattctcttttaaaaccaggaaacaggttcatcactgttcacgaagtagcccaacgtcacactgccaccaagctggaactcagttctctggcatctaaacgcagaactacagaagacacgtttctttccaacgatcagacgtccaagttcttcaagcttcctctaccatcctatttagcattcgcaaacttctaccttgaattgtagctattatcccttccaagatccatctctcaccaggggagcccctgacactccctccctccccagctgggaagccccccaggatgaaggaccgagtctccatgcttctcggtggctgcacacttagaatgggggcttggcaccaggcgagggtctcgcccctttgataccctttgaggttgatttactgccttgcgaatcccgactcccaactgggccctttgaggcgccatgtgcggcccctcgagccagactcggcgccaggagaagggagtcagcgtctggaggggtggacacggcggcgcgcaggagctgtttccaggacaaccgagggccgccccacagtgtccgccccacagtgtccgccccttgcgtctcgccgctccgccccttcccggcgcgggtgctccaaccagcccccgtgagcgaatagagagaggggtgctagatggactctcacctctcagcgcgtggtgcatgccgcccacgccgctgtatagctccagcacccgcaggggctccatccccgcgcctcagccgccgcagccccggagctaggcctgccggtccgtcgctcctcctccctccatcgcgccgctccctcagcctctggttccgcggcgtttcagttcccccgtctcctaccgctccaggctcagggcttcagagggcctcgcggcctggggattggcaagggtctagaggggaacaaaggacggaaaacaataaggcttcccgaatgaatggcagccactctgtagcctgatttacgctccatttccttctttggtgtcaggggccttgattggaaaagaaatccagagttcagtaaaggggagaaaactgccttggtttccaagtgaattaacactggttagaagccagaaaaaaatcatttttcagacaagctgcttttctgtcacttagaagcctgaacattcaaacattctttccacagtgtgccttgtttcctgcctgccctgggtcaatagagagaaattagaccagcctctcccctagagaagattccagaattataagtaaggacacaagtccactcgcagcagagaaagccctgtgtgcgtgctgataatacagaggatgtttgttgtgtctattagtggggttaattatgttttagcttggaaattacacctacatagaatacacagtgaatatcatgtaggattatttatgggtctttaagtgctatggccttgattcctcaggatgatccagtaaccaactgttagaggctcatgttgacgaaaaaagccaaactccgtaaaatgtttgaagaggtttattctgagcacaaggtgaggaccgtgacccatgacacaaccccacagggtcctgacaacatgtacccaaggtggttgggctacagcttgggattatatggttttagggagacataaggacattaactggtatatggaaggtgtacttagtttggtccataaagatgggacaactcaaaaaggggaaggattccaggttacaggtggattcaaagattttctgattggcagttggttgaaagagttaagttgtgacctaaagacctggaatcaatagaaaggaatgtgtctgggttaagataaggtggagaccaaggttcttattatgtagacgaagtctcatacgtaacaggcttcagagagaataattggcaaatgtctcttatcatgtgtaaaaagatgccagactcttagttaaatatctcttggatcaggaaaagacctggaaagggaaagggagtctctatagaatgtagattttccccacaagagatggctgtgtagggccatttcaaaaagtgtcaaagaaatttattttagggtaaaatgctttgatttagggcctgctatctttcatgtgatgctatactagagtcatgttggaattttggtatcttattgctaagaagagtctgttatgtcggtatgaagatctcttgttttaataataatactggtcagttgtgcctgaactccaaagagaggaaagtataatgaggcatgtctgacttccctttcccagcatggcctgaactagtttttcaagtttctttggaatccgcttggtggaggtgttcaatcagtcagttggggggagcttagaaattttgtggggggagcggtgtttacagtcaattataaataaaaataatgtacccatatggaggatagaaaatgttcccgttgtgaagtgtttttctctttgttcccaacactgccacaggtttttgttagggggaacttgggggtaccacttgtaataacaataactgatttgttagaaatgttgcatgcctgctaatgtaatgctgttaacaaataaaagtataaccactgcatccaaattataacattcactggttccttttgatgtggtctatgaaattaactttttaaattgtacatgtgcattggccacgtgctttgctttttgttagtcactttcagataagaagaggtgtatggtagaaagataccagataactcagctttagccctggcgttgccagttaactgattgtgtgactttgagatgaccctggtttcctcatctgtaagataaagcagaagggaacagaaagagatggattgttgagggtgttttagttccaagaaatgaattcatggctgaattcaaattagaaaaaatgagacctgatttggcaacgtgttaattatttattgaatttgaaatatgtagaaagaagacttgttacctgctttaagcaaatgtactcttaaactaactcagcttacagaaaccatgtacacactgacatatgtataatgcatgaagcaaactttttaaatatttattacatatagtaccagaaataaacatcagtacataaaatgtaaagggaactatatagtcacagtgatttatatatagccaaaaaccaatatattaaacaaatctggaattttcagcattcgtgctataaatcaaaataaatgtgacaaaagaccttacctgtaggggagagacagtacagacacatggcagagttaatgaaaaataccaacctcctgagaggaaggagcctcaaaccccttccccacacaggctgattacaaactgtttactctgatacttaaggatcttcacggtatagccctgaacttacttttcttccttaacttttttctcttcttgaactctgctgtaaatgaaactacttggtgttccccaaacatgcccttcagttcacttctctgcttttgttctttcccttctctagaaatctcctagacttctttaagacctatacttcaagtggaaccttccttctagactcccagcctaaaatatcctgacacattgcaactcctaggtctccctcatcttctgaaatacagatcacccttgtacttgtcattcaatccttccagttattctagcccaattgtcacctcatatgtgccacatctttatgtattatttcctctctgaggcttatctcgtgataattatttcaagaatgaaaactctctccataatcataatttacaactatctccatggaagcatctgtagatattaggccccctgggattatgtatgcaaatgtcttccaatcactggcacaaattacatgatttggtggaagagtgggagggctggtggaggtggtacaatagggagtagctgaggagaaaagtgaggtgagcagaatgaaagtaaatattgattactttttccgaaagcaaatcccgaaggctcataagagagaagaaaaactctctagtcccataatttcccataaagtcatcctcaatgatatgtacaatactatttatattttaattagagaaactagacatacttttaaaaaagcaagcaaatatcaaaactggaatgttagtatatttactacattttctctaaaagcagaaaatgcgtttctttaaaaaaaaaacacattaattaacacctggaaaataaaggcacatataaagcatatcagaaataattaaggggcacatcacaatactaaggaccatagtaaaagagtaatagcacacactcaattattgaaggcactgggcttggaatctaatgacatttttatcttctacccttatgtgttctttcactgatcattaatatctaattaaatgaaataaagttttccaagtgctgaaaatgctttttcaatcatctttgaatttccgtgatccaggccactctctttctcagcaaaacatgatgtggattcctatacaggctcgcttacagtctgaggacaccctgcttatgccaatttcagtcatgaataagttgcattctggctttataccaaaaaagaaggtctttatgaattgaatggaaaaagaaaaagtcatatgagaagaaactgactataagaaatgtcctcaagagagtaaattttacaaatttgaattggaaagaccaacagagctcatttaccacttctgagctcccacttatttgagaatgccttctactattttcctgatccatgggcaataagcctcagcttgcaaactgctaatgaaatgtttcatggtgaacagctggaattattagaaaattcttccttatagtctcctaaattccagtttcttttagatttgccctctgcactgattttaaagaattattcctcttctggctgggcactgtggctcatgcctgtaatcccagcactttgggaggcagaggcgggtggattacctgaggtcaggagttcaagactagcttggccaacatggcgaaacccatctctactacaaatacaaaaattagtcggttgggggctgggtgaggtggctcacacctgtaatcccagcatttgggaggcagaggcgggtggatcacctgatgtcaggagttcgagaccagcctgaccaacatggtgaaaccccgtctctactaaaaatacaaaaattagtggctcatggtggtgcatgcctgtaatccctgctcctcgggaggctgaggcaggagaatcactgaacctgggaggcagaggttgcagtgagccaaaattgtgccattgcactccagcctggtcgacaagagtgaaactccatctcaaaaaaatatatatatattttttctcttctataatgatttgctttgtggttaagaatgcaagcttcagagccacagtgtagacaggaagcacacgtctgctgctgcttctccctagctaggtgacgtggagaaagtcactacatccttttttgagccaaagtgcacctctaaaatgtataaataataggacatgcagttgctatgaagattaaaagcccttagcccggtgcctagcacaatacaaataaatgtatgttaaataaagagtgcctgtgcaatgacatcctctaagtgatgcattctcagtcctgtcggctatgaactatcacttggcatgattcagatccctcgcattagtcacagtccagcttggtgatacccattttaggagccaacactgcatgaaacaaactgaacatagatcagcatatggtagagcgaaccagatggctacagacattcaagggctctagggcagctttaggccctgtaggaagtgttcagggttcctcggcttagaccatcgtggagtaaagactgaaagtctggaagctacctcagccctgaccgagtaggtgctgagcatggaaataaagaagtcagacaatagacagcagaattcatagtaaaatagataccgcttaactcttaacttccagaagaaccaggattgctgtgaaggccagagctacagctctgacaatcttcggggaaaattttttcagtgaccttaaccaggatgcatgaactggggagatctggggtgcagaggaggggaacctatcttatagcagctagtcatgtgttgactctttcatacgctaggcactgaggagatttgcaattaaaaaaatttaaaaagcagccggagtggtggatcttgcctgtaatcccagcactttgggaggcctggatgacagagcgagactctgtctcaaaacacaaacaagcaagcaagcaaacaaacaaaaaacttacaaagtgcaaaactttataatctatttcaggccataaaagtgccaatagtaaaaaatgcaattgaatactttcatttcctaagcataggggattttttctaatttaattgattctcactatccaataggataaccagtagccacatttggtaaattaagtttaaattaatttaattaacattaaaattggattaattatactaggcatataccaagcgctccgtattgctcagcacagatgtagaatatttccatcatcacagaaagttctatcgggcagagctgctatgcagtgatggtcccttttagttacagcctttattaatagcccatcaataaatgaccagtgttggaatgtgtgcatcaattattgcatactgagcaccaattttaaataaatgtcccccaaaatatggggcaaaatgaactcaaattaaagcaaaccttacagctaaacattctttctctttctctatcatccagtcaccttactgataaactgtcatggaggaaaagaaacaggaacagcagatgtgttctgtcagtggatgtgataaactttcaagagaaaaagaacagatggaaaatgaacttcatgatgtctttaaggagtagtacttttttttttttttttttttgagacagagtctcactctgtcacccaggctggggtgcaatggtgcaatttcagctcactgcaagctccaccttccaggttcaagtgattctcctgcctcagcctcccaagtagctgggattacaagtgtgcaccaccacacccagcttttatatttttagcagagacggggtttcgccacgttggccaggctggtctcgaactcctgacctcaagtgatccactcaccatggccccccaaaagcctgggttacaggcatgagccaccgtgcccagccagaagtagtacttttctgttataaagtatattctagtgacttctgactgtgaataatgttatcttacacttatctcagaaggaataaaaagaaaacatgtttttaaaaattaggatcatgaatcatgaataaagttaattatacattttctgccaaataaaatagaaaccatgccgtttgcctcatactttaccatttcaaactactcatagatttttaacatggattttgctcttggaggctcattctgaaattctcaatttaaaaatattttgcagcaaatttaaaagatgatgtttcaaagtaaatatagtgggatccattctttaaaactgctttcttaacttcacagaaaattagaactagaaagtatcttaaggactatttgatacactgttttttaaactatttttagagataaagccttttgttcaaaccaaatctcctctggaatggggagcaaagatagagaggtagagcaacagctacactgtaacttagtataaaagatactaagtagactcagttggtcctgcctgctgcccctctacacccaggcaaggccagggatctaaaccaaagcaatcataggagccacaatatgtcacttcctgagagcaggctgacctgaggactgaggggaacttcagagatctatgaagcaaacttgatctgatatggtaatgcatgctataaaagccagacaaaaatgttaacatagctgcacaaaatagagactatctcagaaactaatgtttaacattgataacagatcccctgcccaatcactcctgcaaagacagcttgatacaggacaaagaatatggtccttagaactagatggattaccatggatcctgcctcaaatactccctttcttcacatattaaacgggaaatatttgataacaaggtaactgtgtgtgtatatatatatcatgtgtaaataattgtacacatatcaattatatatgtgtacatatcaatatatacatatatcatttatatgtgtacatacctatatatacaatgtgtgtatatagacaatgtatatatgtacatttcaatataaacacaatgtatataagtgtgtatatatgtatatatatcagtatatacatatgtacacacacacactgatagatatatatcaataatgtgcctagaacaataaatactggtttcctttcactttctgaacaactttgtcataatatagaacccatttcttctaacaatagggagaattattttcctagtatgttttcataaagggtatactaccaagagattaatttgcatattgcctaacaagatgtctttctgtattgttttctattatagccttcaatcttttcaggtggaaaaacacgacccatgttgagaatataggagacagaatttagtaccttggagagctgttagtactttgctccttttctcttgtaatttatctgaacatttagaactgctcaaggtaaccaaagaacacaaatcagacctactcacaccaggaaagggaaaacacacatacacatacacacatacacaggcacacactccttcatagtcttttttctgcattgctacatcatcagaactgataaactaaaacccagaaaaaattcaaagacaaagacttttaattttaccctacttaggtactagtttaggtagacaacagtatcacttagaattgttctcatatgatccaataatgacaaatataactaaaatgtaataacactttcatagtactgccatgactgtatctattcactctgtaagaaaaaaacttcctattatttaaaattattaaaacgtttcttgtcagtttcaagtcacttacgtgagcatattacaacatacaagcacatttcatttttcaaactttctcctctagcctcatgctctagaggctgtcaacctttactttacagagttaaacttattcctgcagatcaagactaaaatgttttatttccatctaaggtaattactgctaagttaaaatgtataaatcctccaatgggattggtactatgtttagtcactcagagaatactggctgatgaaaatagaccacattccaaaagacagaaaaagtatgtttgactaataggaaaataataataacacaataatgacttgtatgtttcaaataactttataaatgtttaaaatgcttattaacccattttagacctatcggcttttctttcttaatattcacttttttacaaataaaaagtagaaaacttcatgtcatttgctcaaagtcacataagttaacattctgcaggttaacagatttatttgcaaatcacacaaacatatttttaaaagaagggaatttagacgataacactagttttttgaggaaaaaataggctggaactcatcaaataaaaatgaaagggagttagtatatatttgaggaaggatttcatattaggagttagacaggattctggttaggaaagaatgtcaggaatctgcatgtctttttttttttttctcctttcattttaggaccaaatcctagaatagagtgaaaaaagaatgcaacaaacagcattatttatatatctttgcacagttttaaaatcaaaaagcagttttatcgtgttacttgttccctcgagagatgaggcattattatttggaaaataaatttttgattatcacctaaagtgaaagggacttaataaacaaacattggaaaagcccaaagtactgaaagctcaatataactgcaagagtctctcttctgaccattttttctcttttttgctaatagtaaaataggcatgcagttgtgcgacctcagctcactgcaacctccacctcctggattcaagtgattctcctgcctcagcctcctgagtaggctgggactataggcatgcaccaccacacctggctaatttttgtatttttggtagagacgggtttcatcatgttggccagactggccttaaactcgtgaccttaactgttctgcctttcttggcctcccaaaatgctgggattacaggtgtcagccaccgtgcctgaccatagctaacattttgaggtttctcccccatagtctttgaactaattgtattttacccaaatcagaatcttattgcatatgttttatacctagctctttttcacctaatatgttatcattatcagttctgtacttcattaaacacatcaaagatatataccctttttaaatgctgactttatttttctgaattttaaaaataatacatgctaatttccaggggtaaaacaaattacaaaagtatacaaaaattaaaggcacttccttctgttaaccttgattccatcctcccccttaaaaaaaattttaagtttgttgaatgtcttttaataaagatgtttatttccaaaactatgatgaggcagtcgcagacatttttgacatgagctcagagttgtacgcatatgtttatgtggagttttcatatttaaaaatatttatgcctgaagtgcaaaaaataataatttaaagaaaatacataatacattttgtttaatttgtcttaagactgatgtattttatatacatgggaatctggaatcatcttacaactgagaaaagttagcagttaacttcctctgaacgttacattttaaacacattttagctcccaaaacaatatgtcaggcctcagatacgcactctttcaaaattctgcctatgaggaaaatactaaatatgaacatttcaaaaataattcacctttaaagctctattttgtgattagactgagtaccttgaaagtcattattacaacccttgcgtgtgtaaagcatcttctcctcagcagcctcacctcctacacgatgatggtctgtaaagtcagggcatccacaaagtgatttattcccccttttacctttaatatggcgctatagaccacattttggccattgcagtgcagtagctcaggtctacaatcccagtgttttgggaaaccagaacagcaggatcccttgaggccagaagttaaagaccagcctttaaacaaaacaaaacaaaacaaaacaaaacaaaaaaatttcattagccaggtgtgggggtgtgtgcctgtagtcccaggtactcaggaggctgaggcaggaggatcacttgagccaaggagtttgaggctgcagtgagctatcattgtaccactgcactccagcctgggtgactagtgagacctgtctctcaaaaagaaaaaggaaaaaaaaaatgagtatattttgcctaaggtacccaaacactagaaacatattccaaagtgaaatatagcagtataatgcaagttttccaaaggaaggggggcagaaatagttacataattcttacaaccatatacttatgttgaaggtataaaagagttgaagattcttgaagacaaagagaaggtctttggagaaagttatcagcatgctatagaggctgtctgcttctcacctcaagcctgagacaaggagattcagagaaccaccgagagagtttaaaatgtaaccatctaacctttgagtttggggaggcaaagtaattcccttggatagatctgagccatacaagacagaaagctgtcttaagccctgttcaaggggtcactcacaagaaaaaaaggtgtcatgcaaaaaggaacaaggggtataccactgaatgactcctaggggctgtggcagcagtcacagagaagcaggacacaagggcatgtctgcatgccagggaagctttgagaaagagaaagagagggagagacagagagagagagagagagagagagaaagagagagagagagaagaaaaccccaaagaagcacctgctagaaaaagggccagctttgagcatatgccaaaccttgaggtgtaaacaccagatcaccaatagcccttgtcaagtaagagttttcttatctcctgcacctctttccctctgtgagctctaatcctgcagtggcaagaaaccttagttagcaaaatgagggaagaggaagggaggcacctaagtagggagagtgagaaacccaggacttccctttcctaaggcacagtcaagccatgggagggaggatattaactcttgggcaagattgaaattttgaaagttacatacatgtaagcgttgtaaattactatgatcacttttctccttagaagtgatcaaagaatggtttggttttaataaatgactggaaaagtcacaggagctgcacaaattttcatttgtaaaatgatcgtttatcaactgctctcttgtcttcttattccttcctgcccttggtttttggacacaggttcttttttctcctctttatgccatagtttaataatatcatcaccaaccatcaacacttattgaggatttactctaggccagacaatcgattatgtattttatatacatttctcatttcatgcacatacaactgttattatcacctccattttttatatcaagaaaatgaggctcttccaaggtagcataattactaagcataggagctgagaaatttaccttggggactctgactccagagtcccactttttttccctgaaagaatgaatgaaatgtattttctcagttcaaaggacacaaagtatcaaatttgcctttttttttaaacaaactctgtcttccagaccggagtgcagtggcatgatcatagcttactgctcactgcagcctagtcctccagggcttaagtgatcctcccacctcagcctcctgagtgactggtactacaggcatgcaacaccatgcccagctaatttttaaaaatttttcatagagataggtttttgctatgttgcccaggctggtcttgaattcctgagctcaggtgatcctcccacttcagcatcttgaagtgctaaaattataggcatgagccaccagacttgctttctttaataaaacagagctctcaaaaaaatggatctcccccatctcatgagaaaattgaaacattccactgaaggagaattaacagaaaagtaaacatagctactttttgtaatcagaaaacttgctgatgtatactcttattccaaacccctggatgatgtcattatgtcaacagtcctttaggattcctcaacccagacataaaataacagtcagagcactagcatatccaaaaatggtattgctttattttttaaaaatatatttcttttttgaaatatcaatcgaatcaattttattctcacaacaatcctgagtaaatatttttaaaaaatatattttttgaggaaacagattaaaagctttaaaaaatgtgattcactgaaaatcaaatagtgaatacatgatagagtcagcactaaaaaggcagatatatcaaagagagactgaaataagcaactcattttttgcatcgacttcgaaacacctctttaccatcagtgtttgtaataatcattcttgatctttatacttcacatttcttggtaatccaaatgcttcataaattctcttacacatcctgaatctcttgcttcattcagcgagataattcctggtgagcaggaatgcatatggttttcagatgtagaaactgggatctccagagacaaacaagttggccatggcaatgggatacaataatcttataggattttacaaacctgatttgaagttttgtcctaatatggaaaggtatgccttttctcactagagagattattctagaattttagagccataccaacatatttcacggtaacctagtgatctggattgcccctctctgagaaagcaaagattctgcaagcaattagtccttcaaatcaaattcctggcttttacaaactgcttctcttttgaattccaaaagttaactaattcttttctatctattcttcatttcctcataagcgaacactcaccagtttccccattttccaaccctatggttttcttattctacgtggccacagacaggaacaattcactagcacagtggttctcagaatcacctggtaagtcttgtaaaaacacaaattgctgggccccacgtactcccagagtttcacattcagttggtctaaagtggggcctaacagttggaatttctaacaaattatccggtgatgctgatgctacaggtctgagtagcacatttggaaaatcacttcactagtacacccccgacgtgttgtcctgatggatccttccaaaaaattgttcagatgccaaggcttgtgaatcgcacacatggtactcaatgcctgggcctcacatcgaaggcttctatattgagaagaaaagaaatatttgtcatgactcattgcctttttcaagctgttccagctacaaaattaaaaccagaaaaggactcaattgaaagcaaaataaaaacagctcaagtaacactgtccaaattttgagacttatggcatgatttctggaattaggacaaaaggtatactagtgattcaggtaaatccttggctgaccacactggaagctggcatctccaaggctcccgacaaaccctggaacccaagccaagttgctcctgcactaccccagaagccagctcaggtccttggcttgtggcttgctttttctgtcaacagtaagttcttccataaagctctccaaacacattagcaacctataactcttttaattaactaatttttctacaacttctcagctatttttaacacagaaaaaaatgtcaattgtcaagaaataaatgctgttgggccatcaagcaggtaggctgtaaatctgaaccgagaacctagccaatgaatttccttaggatttcttccttttgtaatagcaacactcagtggagattttaattccatttttcatatatttggggaaaaaatacggcattttatgtcatagcataaaattcttcagcattccattttagttattaaataatgactagtatttgaaacagcactgttacattaagtaaaaaaaaattggcaagaaatgtcaacttagacatttaataagataatagttctgcttagaaaatgaggtgttagttctgcctttaacgtatcaacatattgtaggaaatactgaccttttattctgatcaccacgagtccattaaatttcaaaatgtgggacagccaatacataatatactactaagtaataaatctatgtcatccaaagggagctttgctgtctgagtccaaaaaaaaagaaaacagaagcataatggtaaggttactgaacactcaggtcttgaaataagtcattctgttatgagcatgagtcaccaaaactacttccttagtgataaaagttgcctaagtaacatcaatgcctccctatgctaagaaggatatgggcagtgtgttgggtttcagacccactagtcatagtcatttcctttacttaattgctaactgcccttcctggctgtagtttggggcctgtgttttaggtagtaacccagcaactagttattatgctatattttatcaaggagaacaaaacagtaaattttgtatttccatggatttcagagtaaaagtcaatgttatttcaccaaggccctctgacttaagggagttattgtgaaactgttcaactaggtctcaatgtggccacatcattatgcaagactcaaaggcaaaatatttaacagagaaaagaggagcagattgggagccctctgagaggaagggaggggtggtattcatttctgcgtccttagcacctagcagggagcctgctcacctaaagattcaaaacgcaggccgcgcaggcgcagtggctgatgcctgtaatcccagcactttgggtaggccaaggaggggtcacttgagctcgggagcttgcgactagcctgacaacacggcaaaaccctgtctctacaaaatatattttaagaaaattatctgggcatggtggcatgtgcctgtagtcccagctgctcaagaggctgaggtaagaggattgcctgagcccaggaggtcgaggctacagtgagctgtgatggagtcactgtattctagcctgggtggtattgcaagctcctgtctcagaaaaaaaaaaaagactcaaaatacatccaccaaatggggtttgctgcttcttttcaaaataatccaatctatgaagtaattgcgccactgctgagttaggatttcatcaggctttgctgttattgatgttgttgttgctgttgagatggagttttgctcttgttgcccaggctggagtgcagtggtgtgacctcagctcactgcaacctccaccttctggtttcaagtgactctcctgcctcagcctcccgagtaactgggattacaggtgcctgccaccacgccctgctaatttttgtatttttagtagagacggggtttcaccatgttggccaggctggtctcgaacacctgacctcatgatcctctcacctcggcctcccaaagtgctgcgattacaggcatgagccattcatcagacttgttaacacatatatgcctttgtttttcccttatatttaccatgggactgtggtatttgctgctgttccccttgcagacatctgaagtatattaagcatgctctattttaaaatctgacatacaaaagcaatgagattagtattgcatgagaaaatttaaaaactggatagtaagcacacatgtattatcagtatcgctgttgggtttggccttgagaatcttggttgcctataattccctgagcctcctctcatttccaaaaactcccccataattcttggattgcacaatagtactaccatgaaggactaaaatgttaataaatctccctaaaaatcaaactaatagatggtaagtatagggagaagttgtttttaactgatataagtattgcattccaaaaatgtggttttatttcttttctactagcaaatggggcaaagcatttttattcacttaatcatatgcaagattcaaatgccataattatccattcattctggtgggttgagtcagaattaagaaatgagatctccacagaaaacccctaaaactgtctatggacttcttgatgattcattaagggcaccatattctctgaatcaaaatgggtcctgttgctgcatcagaataggaaaaaagtagaccaaagagacaaacatcatgataacaatgccaaagagatttggggacatctccaatggtagcacaaaattactatggcatataaagtactacagcacggtgaggtttagagttcggggactggagccagaagtcctgtgttcaggtctgccacttcttggctagataaacttgagtaagctacttaccttctctgtgcctcagtttcttcatatgtcaaaccgggctgtcgtgaggattaaataagttaacccatgggcagtacataatgattgctttcatatgtcataggcactcaatatatgaaagcaatcattacggttatcattagcctttctatccaggtcagactccagttttagcaatcatatcaaagtgtgcctggtgatgattgtttggggttcgctcaaggacctagtattggtcaaggactgtgaaagctagcctagagttagcaaaggaaaaaccctggcctggtgtggtggctcacgcccgtaattacaaaactttgggaggctgaggtgggtggatcactggaggttaggagttcgagaccagcctggccaacatggtgaaacctcatctctactaaaaatacaacattagccgggcgtagtggtgggcacttgtaatcccagctatttgggaggctgaggcaggagaatcgcctgaacttgggaggcagaggttggagctgagatagtgccactgcactccagcctgggcaacagagtgagactctgtcttaaaaaaagaaaaagaaaaaccctgatcaaacacataaatatctctgcagcgtattctatcacacctgtctgaagttctagagatgccattcaagtaaattacaatattgtatcggaaaaacttctcataatttatgtctcaaacaacatgtacttaatctatattaagcttgagtaccacagaatttttttgtccatatgccacatatccatatgagtcacattagagaacatatttctagtacttagctaattagaaatacaaaaccttattcagtggtttggaaatcgtcatggtagaattacaaagagggctgccaaatcctttattctgctagttaactctgaatggtctttctttacttatatctcattgtaatgttaatgaattttcttatcttttcatccttgcatattgtcattatgtcattttatatttccttccatttgctacatggcttcatttagggttactttgtgttattttttttctagaatttgtctttgattttcatttccaacctctgcaaatcacactgatctttgtcactatacacaaaaaccaaagcttgagaggtttattttaaactctgagtttatcaaacatattcacaattaaatataatatttaatatactgaagaaaatcatttcactggctgagtgaatgtgatatgccagagttaaagtatctcccaattgtccatttttgaaaatatcagtcaattcattaatcagttttgaaatacataagatggagttccattatattttgcagatttagaggaaagagattatgactatatagatggtcaataatccccttccaatgtcgcaggttaccataaaggcaaataaaaatttacattataaatttatctgttctttaaaagaaaattaataaactgatatgcacatcaataggagactggctaaggaaaatgtggtacatatacaccatggaatacaatgcaaccatgaaaaagaacatcagccaggcacggtggctcacgcctgtaatcccagcactttgggaggccaaggtgggcagatcacctgaggtcaggagttcgagaccagcctggccaatatgatgaaaccccgtctctactaaaacacaaaaattagccaggcatggtggcatgcacctctaatcccagctacttgggaggctgaggcaggagaattgcttgaacctgggaggcagaggttgcagtcagccaagatcgtgccactgtactccaacctgggagacagtgagactctgtctcaaaaaaaaaaaaaaaaaaagagatcatgtcctttgcaggaacatggatgcagctggagaccatcatccttagcaaactaatgcaggaacagaaaagcaaatactgcatgttgtcacttataagtgggaggtaagatgagaacacatggacacaaagaggggaacaatagacactggggcctacttgagggtggagggtgggaggagggagaagataagaaaaaataactattggatactaagcttaatacctgagtgatgaaatcagccatacaacaaacccacatgacacgaatttacctacataacaaacctgcacatgtacccttgaacctaaaataaaagttaaaaaagaaaattgtgctattaaacaaaattattattaggtcccttacctaaatatcttgagaaaaaaaattctaccactggggaactacatgacctcctcaaagtaggcagagtaaattcccaagacgccaattgcacttggcaaattttctagctgtggcataatctttttctgtctacaaaaaaaaaaaaaaaaaaaaaaaaaaaaggaaaaacaacattataggccactgggcaaatacctgctagttttcttatggtagtctaactcagccagtaactaaactgggatgatgtcattcagggctctttcaagtgacatgaggtttttataactggattgatgggttttttgtttgtttttggttgttactgttttttgatcaagtgaactaatactgaaaatctggctttaattctttgtccaacatcagctggaacttatttttgcactagatttctcctccatttgttaattaaactcttgtgttccccctacatacttaccaccttctctttcctctgtcatagactgtgaattagacattgatgaagttaaaaaatcacttatagtctacagggaattgttagctatgttttccccctcaaatgttcaaagatacctcccagaaagtgtctttaaggtttttctttcccttctgatgttttaaacactgttttagagaatcgtgatgttcatgattttatactagctatgggaacaagactaacacactcaaagcagctaaatgacaataaaagaccatctactttattcctttaagggaagagattagaagtgcttagaaatgctgtagaccctgggctatggtctgatagtggtcggtgggtattagattcttgtaggagcgtgaaccccactgtgaactgtgcataccagggatccaggttgtgcgctctttacgagaacataacgcctgatgatccgtcattgtctcccatcatccccagatgggaccatatagttgcaggaaaaccagctcagggctcccactgattttacattatggtgagttgtataattatttcattatatattacaatgtaataataatagaaatgtttgaatcatcctgaaaccatccccctgcctggtccatggaaaaattgtcttccatggaatcggtccctggtgccaaaaaggctggagaccactgctgcataatgtaaaaccacactatccaattatagctcaccttcatgagactataatttatatagactagagaagggaagatatttccaggttaagggacctgaattaaccgtctttcaactcaaattaactttccaactaattatttctgtctggaatgcatctattctttcttttttcttctttttttttttttttttttttttgagatggagcctcattctgtcgccaggctggagtacaggggcacaatctcggctcactgcaacctccgcctcctgggttcaagtgattctcctgctccaacctccctagtagctgggactacaggcgggcaccaccaggcccagctaatttttgtatttttagtagagacagggtttcacgatgttaaccaggatggtctccatctcttgaactcatgatccacccgcctgggtctcccaaagtgctgggattacaggcgtgagccactgcgcccagccgcatctattcttttaattatcctgttcacaaggctcagagcccactcgatttttcttatcccctagcccttaaacatcacaattccttaaatcatctccgcgtcaggctccctttaccatccacttgatctacctcaagcagacctcaaatcagcctcattctccacccctaaaatacagctacaagcatgtctttctcctagttgaaattcacaatgcattccctctgcccaccacattaattatcaactccttttcctggcatttactcatccaacgcatggccccacgttaactttcagttccctttctcccctacaaatactccataatccagcaaccctgggattattcctgttccctaaatgtcctcagcactctgccacccacccagtttttgcttatactgtttggtcagcactagagcaacctttctttgccatgataagctgtagaaattgtacttattcttcaaatactatcttttctaatgacattttctgttctcccccaagtagataggatcattttttttctcaacaaaatttaacttatttttaggtatagtattatagtttttttttttaatttagatttagggggtacatgtgcaagtttgttacataggtatgttgtgtgatactgaggtttgggcttctaatgatatcgttgcccaaatagtgaccatagtactcgataggtggtctttcaatccttgcctcctccctcccttcccactttgggaatccctagtgtttactgtttccatctttgtgtctgtgtatactcaatgtttagctcccacttagaagtgagaacatgcagtatttcgttttctgtttctgcattaatttgcttaggtttatggtttctacttgtatccgtgttgctacaaaggacatgatttcattctttttaatgcttatgtaatattctacagtgtatatgtaccatgttttctttatccaatccactgttaatgggcacctgagttgattccatgtctttgttattgtgaatagcgctgcaatgattatttctccacctatgctctttagcattttatcctatctgtgctacagcactcacttaccacattctgctatgtattccaactctttacctgctttctaatccttatacaaagctatgagatccctagaggcacaaaaagcaccttacttactgaaatatactaagaaacattgcaggcagtgcaaaaaaatatgaatacatctatctgttaaatggaatatgaggaaagacctgagggtgggggaaatgtactatattcatggaagaagaaggagcttagggttctactgaaattgctgcttgtggggaacagtggaaaatgaggatagatatatatacttgacataacttccaaaggccactagttaactatgatcctcaacctcctctgcaggaaaatttctgtttccctcatagtctttatcaccagttgcaatcattttgtttgttcctttacgagtattgtctctctgtatcatatacatatacgcagactcaacactatgttccaagaggctttgggccatacctgctttggtcccttccccagtgtctaccagactgcccggcacatggcaaatactcaataagtatgtttggagtaaaagaataaaagttgtgtatcacgataccttgtgaagattaaatgcagtcatgtctaaagtgactagcacaatgcttaaacataataagcaccagtaaatattagccccacacctatatttcttccttctatgaactactcatgtattaatgtatatattcgacagatgtgcctggcttttctctagccactggaaatacaatagtcaacaaaagagagaaaaattcctacctgcccttagggagattacattccagtgaggaaatgagcaattcctgccaagaagtttaaagaaggaagtttttggccaaatgtggaggctcacacatttaatcccaacattttgggaggccagtgctggaggattgcttgagcccaggagttcaagaccagcttgggcaacatagcaagaccctggctctacaaaaaataaaaagttagctgggcatggatggcactcccagcttctagggaggctgaggcagtaggaatacttgagctcaagagttaagaggctgcagtgagctatgactgcgccaccgcactccagcctaggcaacagagcaagcctttgtctctctctctctctctctctctctctatatatatatatataagttttaaagaaaaaggggtgatggcttgaagatagatgcattggtggcagaaaagaaagcatggaggggccgggcgcagcggctcacacctgtaatcccagcacttcgggaggccaaggcgggtggatcacgaggtcaggagattgagaccatcctggctaacacggtgaaaccccgtctctactaaaaaaatacaaaaacttagccgggcttggtggcgggcgcctttggtcccagctactgggaaggctgaggcaggagaatggcgtgaacctgggaagcggagcttgcagtgagccgagatcgcgccactgcactgcagccccgggggacagagcgagaccccgtctcaaaaaaaaaaaaaaaaagaaaagaaagaaagcaagaaagcatggaggaacggaagccaattgcagtattgcaggaaggaggtatgaaatggcttggatctgagtgacagcaacgtaacagaggagtggatccaggaagagcggggcatgatacccattggatctgagatggaagagttagttaggaagtggcaatgatgaaagtttccaatcttgttgttttgctgtattgccctgaatagacagggaagctggcaggggaagcgatggtggttcatgggctttagggcgtgagacgagctaaacccagtcttgaatgtgattcctcctaagaggcaaaatggaatctaaatgaaaatttttaactcactaatgaaagtaatgttttagaatgcaggtgaaaacaaatctcccttcttacatagtatgatcactgttacgtcatttgattttgttttttacatcattttacatcgtaaacatggtgttatagctgtctgccaaattcctctaaaaatatgccccttcgcagcaagatctgtgttaatcatcccatctcttcagttcctggaacacagtaggtccctaaatgataagtccagcacttaatatttgtgaattgaactggttgatagtaagcaaaattttgttatcaaggcaaacagaaaagagaagtgcatttagagtcagaaaggcctcgattcaaattcttgtccccttcttcccacccctgctagtcatacaaccacaagaaaattgctagcctgcatcttaggctcctcattagtgaaatggatgatcaagtctatcttgcaggagggtgtgagtgcatcaagttaaattaaatatatacacagtgtctgtcattcagttggcatcgacattacctattatactatttattattattacctgtatttccatggattttaggttttgctatggttttcatgctctcagtgtaccctccttcttctttctaacattccatccaaacagtcctgctagatgctgttctcttggcatggctagccagctttcccaaagcctccataggccactaagcttctgtccacacggctggctggtccgtcagtttaagtcacccaattcaactgacatcatgacagcttgcaaattccaaaatcactgttggtaatttagatgtggtagtgtttatgcctggcctatgaattataccaccacgtctttatttatatgcaacttgcatcatgctgtgttgaaagcagcttcccagtccagcacttgaaaatacttctcagataggttttcatagcaattaaaaaataaacacaaaacccgggaaagctatgtctgtgtacccaaaagatacctaagaacacagtagcatagagagcaggctgagttgtctcactcaaggatgtgagttcagattcattgatctccctgactgaaaactatcacccattgattgaggcactgtatatcttatctcagaatgaaatgaattcttaagtctcatgaattctacatgaatgaatgaaagtgtttctgcaactttcacttctcttgcagcccaccaaaaacaatggcattttctaataggaaaaacacattgaacgctgtatatgtgcgtatgtatatgtgtgtgtagtacatacatatactttattttgagacaggttctcactctgttgcacagggtggagtgcagtggtgtgcacagcctactgcagcctcaatttcccaggctcaagcaatccccctgccttagtctcccaagtagctgggaccacaggtgtgcaccatcacgcctggctttttttggtttcatatatatatgttgcctaagctggtctcaaactcctgatctcaagcaatcctcctgctttggcctcccaaagtgctgggactacagaacacctacacttttatacattctatatgcatcaaacaaacatatgtctgtgacaagagagcagcctatcacagcccagagacttcagaatggggacaggttggaaggaggagctacctatccctgagatgatgaagaggaccagtgcccattccaggagacatcaccgcagccctgaggaatcggctatgggcaccagcagggcacagtgccacacctcgccaatgccttgtcctccttttccatagtgagtcagtcagcaagcgtcgcattgccctgggatcggcactgcacgtagaagtgagttccacactctcttcctcccatagggagatcacttttctcattctaagggttccaggcacactcacaatggtggcatttgctgagcagtggcttgaataaagggctctcagaaagcaagatgtaactcagagcataggcttgtcctccagggcacccagccccaggaatgctcttggggaatgacctgcagcctcccagtgaaagagagaataaaagaaagccccagcaggcgagctgggcagtagagagtcctgtaattccaccttggcaagcaccatttgcaagaacgaactgggataaggtaaacaaaatattgcctaaaagaggcttgtccaaagaagtcagaatacgctcttcatttacctctaaattatgtttcttaaataccaagtaacctgcagtaccccctgccgccaccaaaaaaaaagagagaaatacttttttttctcctatccactgcagaatcaattttacagtggttctgcagttaatcctttcagtacaccataaatctaaatactcaaaaaaacctgtgccttttcaattgctactaaatcacgagaagactgatttacatagtctccttttatctcccttggcgggtaagtactcagctctgctcgttactaatattgaaacaacagcccttgaattgagtgatttccctagaaaggttaaggtgaccgaatctgaacactccctccatgtcttggacacgaagttttttgctgcgtagacagttttatccccctcaccccaaggtcaattgcacgaattcttttggaaaacaggacctatggcatttcccagacaaatcaccgtgaaccctgtactgtgcattgctgtcctaaaattaacacataaatctattgccgccaaagattctgtcatttgtgttacataattgcctttcatttgaactcattaatcaaattggggtttttaagcaacacctaattaattctttaactggctcatattatacctttaatgacttccaccagggtaaaaaccactgatcactgagttctattttgaaactacggacgtcgagtttcctctttcacccagaattttcagatcttgtttaaaaagttgggtgtggtttcatggggggagggggaagagcgagaggagaccagagggacgggggcggggactctgcaagaaaaaccttcccggtgcaatcgtgatctgggaggcccacgtatggcgcctctccaaaggctgcagaagtttcttgctaacaaaaagtccgcacattcgagcaaagacaggctttagcgagttattaaaaacttaggggcgctcttgtcccccacagggcccgaccgcacacagcaaggcgatggcccagctgtaagttggtagcactgagaactagcagcgcgcgcggagcccgctgagacttgaatcaatctggtctaacggtttcccctaaaccgctaggagccctcaatcggcgggacagcagggcgcggtgagtcaccgccggtgactaagcgaccccacccctctccctcgggctttcctctgccaccgccgtctcgcaactcccgccgtccgaagctggactgagcccgttaggtccctcgacagaacctcccctccccccaacatctctccgccaaggcaagtcgatggacagaggcgcgggccggagcagcccccctttccaagcgggcggcgcgcgaggctgcggcgaggcctgagccctgcgttcctgcgctgtgcgcgcccccaccccgcgttccaatctcaggcgctctttgtttctttctccgcgacttcagatctgagggattccttactctttcctcttcccgctcctttgcccgcgggtctccccgcctgaccgcagccccgagaccgccgcgcacctcctcccacgcccctttggcgtggtgccaccggacccctctggttcagtcccaggcggacccccccctcaccgcgcgaccccgcctttttcagcaccccagggtgagcccagctcagactatcatccggaaagcccccaaaagtcccagcccagcgctgaagtaacgggaccatgcccagtcccaggccccggagcaggaaggctcgagggcgcccccaccccacccgcccaccctccccgcttctcgctaggtccctattggctggcgcgctccgcggctgggatggcagtgggaggggaccctctttcctaacggggttataaaaacagcgccctcggcggggtccagtcctctgccactctcgctccgaggtccccgcgccagagacgcagccgcgctcccaccacccacacccaccgcgccctcgttcgcctcttctccgggagccagtccgcgccaccgccgccgcccaggccatcgccaccctccgcagccatgtccaccaggtccgtgtcctcgtcctcctaccgcaggatgttcggcggcccgggcaccgcgagccggccgagctccagccggagctacgtgactacgtccacccgcacctacagcctgggcagcgcgctgcgccccagcaccagccgcagcctctacgcctcgtccccgggcggcgtgtatgccacgcgctcctctgccgtgcgcctgcggagcagcgtgcccggggtgcggctcctgcaggactcggtggacttctcgctggccgacgccatcaacaccgagttcaagaacacccgcaccaacgagaaggtggagctgcaggagctgaatgaccgcttcgccaactacatcgacaaggtgcgcttcctggagcagcagaataagatcctgctggccgagctcgagcagctcaagggccaaggcaagtcgcgcctgggggacctctacgaggaggagatgcgggagctgcgccggcaggtggaccagctaaccaacgacaaagcccgcgtcgaggtggagcgcgacaacctggccgaggacatcatgcgcctccgggagaagtaaggctgcgcccatgcaagtagctgggcctcgggagggggctggagggagaggggaacgcccccccggcccccgcgagagctgccacgcccttggggatgtggccggggggaggcctgccagggagacagcggagagcggggctgtggctgtggtggcgcagccccgcccagaacccagaccttgcagttcgcatttcctcctctgtccccacacattgcccaaggacgctccgtttcaagttacagatttcttaaaactaccactttgtgtgcagttgaaggcccttgggcacaatgagagccagtcctccaaactttcagaaagtttcctgccccttctggcaggctgccaatcaccgggcgggagaaggaaggaggggaaggcggtggagggagcgagacaaagggatggtccctcgggggcggggatggcggggctgtcctgtaggtctgtgcggccaccgtgattgcccctctgcgcggtgcccgaagtcccgctgaaacctgccgagggcagcaggtctgaaagctgcaggcgctagttgcgcggaggtggcgcagctgctctggaggcgcagagcgaatacgtggtgtttgggtgtggccgccccgcccctggcggtttcctcgttcccctttggttaatgcgcaactgtttcagattgcaggaggagatgcttcagagagaggaagccgaaaacaccctgcaatctttcagacaggtttgtagactctcttcccactcgcagccgcctgaccccacccaacacaacccacgagcaattctaaaagttgcttaactcacgtctaaaaagtgcaaaacttcagggctgcgcgtaaagccctctagtggcgggaagaccacaggttggagcttctcatgattagaaaaatattaataaaaccccttgagcgattttttttttttttttgagacggagtcttactctgtcgcccaggctggagtgcagtggcgagatcttggctcactgcatcctccgcctcccgggttcaagcgatccttgaatgatttctaagcagttccttgggacataaagaaaaatcttttaactttttactttgtttcccaaatgttgcacagttttgcaacacattgaccttctggtttcgaacggttacaattttagattgtggtttgccaaagtcaagttgcttaatttttacaaggccacaaaaagcgcaattatgccctgcagtttaaaatggaaaacgtgttggaagataagaaaacttagtttccaactggaatggagccagcaagtttcttttcttctttgcaaattctattgtgtcattaaagttcgatggaagtatcactatgcacaactattttgtgatctaataagggtgaaaaggagccatctgtccccttggctaaggggtattaatggtttctatgggcttcactatggaatgtagatacagacattctggcaaatgtggtggctctggacagaaataataggagtctttgtattcccagggaagctttgcaacaggctacattcttactgaatatgtaatgatgtaagcacggttctaattggacacaagtatttgctaacatccgttatctaatatctggcccagacttgagaagtaggtaatgtaaaaagtttttaaagctacaagcatacctcacattttaaaagtcctttcttgattgggttcttgtgttctttagcactcttgccataaaaaataataacagtaataaacccaaggctgaaaaactgaattttaactaaagggtttttgtgcgtgttttttttttttttcaccaaaattagatggacttacagaatttttaacttaaaattggaatccaaaagccagaagatccccattatagtttatagttgtattccctggaatatttactgggattaactgcaaagcactctcagatgaatagtgtagtataacattttgaaactgaaatacatttaccaaattaatttaaccacagcaatgtgtgtggttcattttagtccttgagcatttttgattatcatacctgtcatgttttctgcagtgtagtgagttaacataaaacaacatcaatacaaaagatcctctgtttcgagattaagcaaaattcctcattctcttcaatgtgatagaataccacattgatctttctttggaggttagtaaaatatcttttatgtatttttcagggcttaacaagtaaaaatcaatgttttcatcaagtctgatctttttgtcacccactcttcattcatttttccactaaggtgatagaaaagtctcaacagtttaagaccgtaaggctatgaactccaaatataattgctgacaagataagcaatcctcacgcatccttttgagaggaaataaaatcttagttgcaagattacatattctgatttggaatgctgagctttttaaatggaaatatagaaggacggctgaatcagcaaaaatcctttatgtagtttcattctttgcaagcttgaccagtcattctgaaacaggctaactgaactgatacagtggcaagtgaaaaagacatgcctttacaggatgagtcaaaggagttttagaagaaaaatccaccagagaaagccaagcaaatacagttcagagttacatttcttttccattttttcctgaactgaatctttggcatgcatatcctgaattgggttattgaatataaatctagccttgtacaatggatgccagatgactacatatttgctttggagcctaaggataagtttcaaaagatttgagtggagaagaaaagctaaaactcttgaagcacaagtttctgttctccatgtactcaagtgtacatgaagttgtgaaaatttgtccacctctatcatcatgttattccatgaaattacaaaacaaatcttaaaaatgttgtggcatagattttctagatttaaaaagtaattaaattaaatgaattactttattttttgagacagagtgtcactctgttgcccaggctggagtgcagtggcactatgttggctcactgcaacctctgcctcctgggttgaagaaattctcctgcctcaacctcccaagtagctgggactacaggcatgtgccaccacacccagctaatttttgtatttttggtagagacggggtttcgccatgttggctaggctggtctcgaactcctgacctcaagtgatccacccgtctcagcctcccaaagtgctgggattacaggcataagccaccatgaccagccttaaaaagtaattttaaaatatcactggtaaaatgtggattcagtcatgattgagtgcagtttaccatgtgtgtggacatttatttattttaaaattgtctgatcaccaccttgagtaaaacacaagcagtcacaattaaaatatattagtgagcaggagaaagcacagcatattatagcactgaatgatttataaacctattccagggtcataaaatgtgtcaacggcttttctatagtaaggagactaggttcagatggttaatctaagacaaataaatgagataagccatacacttttacatcctccatgtcctgtcttttctctgttcaaaataggatgttgacaatgcgtctctggcacgtcttgaccttgaacgcaaagtggaatctttgcaagaagagattgcctttttgaagaaactccacgaagaggttagtggagtgactttcggggaatgaatgagggtaaggcagcccccacggttggcagagctgaccgtctgtctgttctttttgcaggaaatccaggagctgcaggctcagattcaggaacagcatgtccaaatcgatgtggatgtttccaagcctgacctcacggctgccctgcgtgacgtacgtcagcaatatgaaagtgtggctgccaagaacctgcaggaggcagaagaatggtacaaatccaaggtaggaaacaaatcagtgcggcttcaaccaaagaaaagcattgtgttctcaaaaccccatacctgtgtgtgattcctaaatatcctctagctccaatgcaaagctggctttgacttcttgctcatattgtgtttgccaccacagcctccccaccactcacatcacctcctttatttatttatttattttcttatttatttatgagacagagtcttgctctactgcccaggctggagtgcagtggcaacatcttggcacactgcaacctccgcctcccaggttcaagtgattctcctgcctcagccccctaagagctggaaccacaggcaagcaccaccatgcccggctaatttttgtatttttagtagagatggggtttcaccatgttgaccaggcttctctcaaactcctgacctcaggtgatccaccctcctcagcctcccaaaatgttgggattacaggcatgcgccaccacgcctggccacatcacctccttcagaatagcagactctcttccccctaaccttgcctccaagtaaaccccaatgccatacctttgacctccactgtgttgaaatgagcactgtagagtgaactctgaaaatactaatgtcagtactccactgctctttccctggctttcaaaacagaaatttaaacctatactggaagacattcagtgagaaatatgattttttttttctaagagagtcaaaagacttgaatgtgagcaatctacatttctgttttcttcccaacagtttgctgacctctctgaggctgccaaccggaacaatgacgccctgcgccaggcaaagcaggagtccactgagtaccggagacaggtgcagtccctcacctgtgaagtggatgcccttaaaggaaccgtgagtaccaaccctgcagtaaaagagggaaaataatgacccattctgctgactaggctcatgatgatacctgaacaaaatgttgagtgagtaaaaatgtatatcataatgcaaagaaaatgagttatcaagacagactcaaaagggacttcatggaactcttgaaggttttagcttgcctatatcattgcttctaatatgaaggacttggtactcgcattctccacctaaaattagagtggtcgccatttgccgctaatggaaattattgcagaaggtctgtaaatggttctgggaacagctgggtttttctgagaaataacaccagacatctttctcaccccctgcagaatgagtccctggaacgccagatgcgtgaaatggaagagaactttgccgttgaagctgctaactaccaagacactattggccgcctgcaggatgagattcagaatatgaaggaggaaatggctcgtcaccttcgtgaataccaagacctgctcaatgttaagatggcccttgacattgagattgccacctacaggaagctgctggaaggcgaggagagcaggtagggaactcagacttggatgcgtgaactaatggtgaccatttgttaggccctgtgccactgggctctaagcagtgtcacatttaatctttagaaagtttctttgaggtaactgctttccactttttgtagaggaggaatttgaattgagagagagtaagtgacttgctgaaaaagggttaatcaacagcagagctgggatttgaacccataactctgtcaaagcctccactcctaactcctgttcatgctcctgtggagaaaatgcttgtagtaacatattttaaatgtactaacaagaccagtcatgggaaaatgtttctgagacaaatctctagtttatgatttaaaacagtacgttttcttacgtgacgaaaacaaaaagtgtgttaatttgttcccagtggttgaagttatttgccaacaattttactgtttctcttcatctgtttataggatttctctgcctcttccaaacttttcctccctgaacctgaggggtaagcattttatttccctttaggaaaaacgtcagctgcttgtaaccactgtgtttatgtcaaagcattcattttttttaggatatctgaaaaaatgccatataagagaaaactctataaaacatctataattttcgaacccaagtacactcttgcattctatgctttaagttaaatgcaaactcctttttccttcttcctgctgcaagtactatctcatcctgatgctcaagagtgtcagggcctgggtttccaaacagagactaccctaaaattatttggcgagtagtactttacacaattgcctctcccccacaaatcataattgtttcagtaaaatggttacttggtttttccaagaaaaaactcgtttttactcatttttggcctgtttgtttatttagaaactaatctggattcactccctctggttgatacccactcaaaaaggacacttctgattaagacggttgaaactagagatggacaggttggtatcttttaaggaaaaaatagggtaatctcagacaggagttgatatattttaaaatcagtgaatctgaatctcagatacagctggctaatttgagaggttcaggtttcattcatgcctactaaaaaaagaataggcttcttcttccagcagtacacacagccaactaattatttggctcctggatgtgaagttgagatagcagtcttcctgtgctccagaattagtgatttgctttggtgcttaatttgaagtgggagtaagcttccttaaaccacttcctaaagcagctacatgaaacagcttcactagactacctcaatatgaggaatgttttgatcctggacatatggtgtcttcctacctccatactttatagattcctaaacccatctatataatacaagcatgtgccatacgatcatttagtttcttattacctccctatgccaggaaagaaatagttgcaatttattgtagtcatcatgaaatcttcccttgcacataaatttaaaatgtacctgctgcacattttaatatgtcttaattgcttttaaacttggctgtattgtgtacaactattataccatcttttataaacacagttttttaagaaatttctttttgtaagttacaacattccactggatccttatattgcctgtagtggaagagggtcttgtgtgtctgccccttctagttttcactcatgcagaagcaacataaccttctgatttgcacaataaattacatatatttagcaggatttttatttgccgtgatatataggataatttagtctttggcatgtggcattatatttattttggtttttttttttaaacaggttatcaacgaaacttctcagcatcacgatgaccttgaataaaaattgcacacactcagtgcagcaatatattaccagcaagaataaaaaagaaatccatatcttaaagaaacagctttcaagtgcctttctgcagtttttcaggagcgcaagatagatttggaataggaataagctctagttcttaacaaccgacactcctacaagatttagaaaaaagtttacaacataatctagtttacagaaaaatcttgtgctagaatactttttaaaaggtattttgaataccattaaaactgcttttttttttccagcaagtatccaaccaacttggttctgcttcaataaatctttggaaaaactcttttgttgtgttatttattggataatatctaaacaattctctacttggtcctattagttaatttgtcattacaatcatgtaagttgataaattcaggttatttatgcttgagatgtagttcttaattttgtcatttttgatagacctcacttctttttattattacttaaaaacatttacaaataggtggtgtcgaataaaataacttgtaccaaaatgaagataggtctctctaaaatgagctcaggtctgtgattttaatcataacaaacagacttcctaaaattaaaaaataaaaactttttttagtacatatagcatatgagtaacacaaattccactttgggagttaaagcataggaagttgccaagatataggggcttatcttccgctagcaagatgcagagaaatggaaaaagttcaccaagtttttcttttatttaagacaggatctcactttgtcactcaagctggagtaaagtgactcaatcatagttcactgcagccaacaactcctaggctcaggcaatcctcccacctcagcctcctcagtagctaggagtactcaaaacaaataaacgaatgtctaactttagcctaaaatgtgagttgtcttcccaaaactagccacacacctggctaatttttttaattttttgtagagatggggtctggaagtgttgtccagcctagtctggaactcttgtgctcaagtgatcctccctcggcctcctaaaaagctgggattacaggcatgagccaccacactcagcctagttcaccaagatttaatatatgaaaccgttctgctttattgtgtgtgtgtgtgatagctatatctatatccatctatctatagatacagatatttgtaaagacctttgtgtttggagaatggcattaggcacaaaataaaacctcctaccttttcttccagggagatgagattattacatgtgaatcgcatggtgctaactgtaggtgtaataaggtttcaaagtctggagcaataaaaatgaagtggggcagtcatggaaagctccctagaaagcactcagtgtgagcatagaacttacataaatagacgagagaaggtattgccaagaggcaatagtgtagtaatggtcaataatgtgtgttctggagtgaaaacttcgagtacattctcggacatctctaagtttcaaattatggaaatattattatcttctctcctccgagaatgatgtggtagacattgtcataatttttctgtgtttgcatctgcctctctctggagttaactagtgagatggttctcacaaagccctgcccatttgcaggaatgttcactgtgattttcatcattttggttaccgtgggtactgcatgaataaagaaaggattggggctggatgcagtggctcatgcctgtaatatcagcactttgggaggctgaggcgggtggatcacttgaagtcaggagttccggactagcctggccaacacggcgaaatcctgtctccacaaaaaatacaaaaattagccagccgtggtggtggatgcctataaccccagctactctggaggcagaggcagtagaattgcttcagcttggaaggtagaggtttcagtgagccaagactgtaccactaaactccagcctgagtgacagagcgagatcctgtcttagaaataaagaaaaaaaggattggctgctgaagtaactcctagccattctaaaggtaaattctaaatgtaaaagtaaactttacattctagtttacttgggacattcttggtttatgactgttgtgtaagcataattattaataataacagtttggacaatagattgtatgtggtcaggtcatgctacccttagaataatcttacagaaaatgtgtctgtcccctcagcccccatattcaaacaactgacatggacaatggcataattaattttattagtgtaaacatccaggtctttgggcccttatgaaaccttttctatttgatttttcgttttgctttctgttctttagccattctcaccactgttccccgcagcctcctaaccctaggatccctgtctcctcttccacttgtatcccacctttggcttataagggtcatccagggaagaccccagccccatattggctttcttcacacaccagactcattattatttttttaaaaattgaaaaacaagccacagttttctttcaggggaaaagaaccaggacagcaagttctgctcatacctattgagattagtagactcccactcttaaaaatcctttacagatggaggccaggcatggtgaccaaggtgggtggatcccctgaggtcaggagtccaagaccagcctggccaataaggcgaaaccccgtctctactaaagacacaaaaactagccaggggtggtggtgcacacctgtaatctcaactactcgggaggctgaggcacgagaattgcttgaacccaggagatggaggttgcagtgagccgagattgtgccactgcactccagcctgggcaacagagtgagactctgtctcagaaaaagaaaaaatatcctttacagatggaaacaacagacactgggcacttcaaaaaggaggaggttgggaggagggcaagggttgaaaaaccacctgttgagtacttgggtgatgggttcaatagaagcccaagctctagcattacgcaatatatctatgtaacaaacctgcacatataccccctgaatctcaattaaaaaaaaaaatatcttacagctctttctaaagtcacatcccagaaatggaaaagaggagtgggcctcccttatctgtgactgtcagaaattagaccataatgaaaagcttcacaggtgccccatctccgaagaattcaaatttatttcagtcttcttctattttatctttaattatactgttagtgtttttccttatcttccactttgaaaaatttataaaaagaaattatgttctcaccacctatggtgaaagagagattaatgcttggttccttcatgtataatattaatccaagattaaaggagacggaaaaagtagggggatggggagaaagaaatggtggcttaaagtgtttaagtctgaaaaaagttactaaggggatggtgaaatccatggaactcatgtgactgtcaagtcctactaagttttctcacagtgacagatggaaaaggctgctggtgaatgaaacaaatgtctacttttagcctaaaatgtagtgagttgtcttcccacaactagccgctaacaaaggcgctaaaaaaataaaatctcttcccaggctatgagactaagggagtgagtgtgtgtagtgatgggggagggagacagactttcactccaactatcgtctgaatgtttgtgtccccccaactttgtatgttgaaacctaacctgcaacgtcatggtattcagatatgtctcttgtgaagtgattaggtcaggaggctgaagcccttatgaatgagattaatgcccttataaaagaaactccaggctgggcgtggtggctcacacttgtaatctcagcaggtttggaggctaaggcaggtggattgtttgagtccaggaattccagaccagcctgagcaacatcgtgaaacgctatctctacaaaaaacaaaaaacaaacaaaccatgtgtggtggtgcacacctgtagtcccagttactcaggaggctgaggtaggagaatcacttaagccccggaggcagaagttgcatgagccaagatcatgccactgcactctagcctgggtgacagagtgagaccctacctcaaaaaaataaaataaaataaaataaaataaaacaaaaaagcccataatcccaacactttgggatgctgaggcaggaggattgcttgagaccagagttcaagaccagcttgggcaacaataagaggccccatatctacaaaaaataaaaatataaaaaaaacaggcatggtggtacgtgcgtatagtcccagctactttggaggctgaggtgggagaggatcacaggaacccaggaactcaaatctgcagtgagctaagaaggtgccactgtactccagcctgggtgacagaggagaccccatctcaaaaaagacccttaaaaacccacttgtcccttccatcatgcgaggacacagcaagaaggcaccttctataaaccaggaagtggccctcaccagacacggaatctgctggtgctttgatcatgaacttctcagcctccagaactgtgagaaatgtttctgttgtttaaaagccacccagtgtgcatgattttgttacagcacccgaaacagcctaagacagctccatcctaaggattactcatgagctttgtgaaggaagcaaggataaagcagacactatgcccacacgaagtctccctctccaacctgccagaccagctgcatgaagaagggtctgtactactcagctagttttactgtaacagaataccacagactgggtggtttaaacaacagaattttattgtttcacagtaacagaggctggaagtccaagatagaggtgctggcagggttggtttattctgagcctctcccattggcttgcagatgacacgttctctctatgacctcacatggtcaccccttagtctgtttggtctgtgtcctagtgtcttcttctaaagacactagtcccattggattagggcctacccatatggctccattttaccttaatcacctatttaaaggccttatctccaaatactgttacaatttttttttttttttttgagatggagtctcactctgtcacccaggctggggtgtagtggcacaatattggctcactgcaacctctgcctcctgggttcaagcgattctcctacctcaacctcccaagtagctaggattacagctacacaccaccatgcccagctaattcttgtatttgaatagagacagggttttgctatgtttgccaggctggtctcgaactcctgacctcaagtgatctgcccacctgggcctcccaaagtgctgggattacaagtgtgagccaccatgcccagccctccagtacattttgaagtactggaggttagggcttcaacatatgaattttggggaaacacacttcagctcatactaggatcaccactaaacattgggaaacactctccttgtctgtactcacaataggtctgtgatacagattttcaaaagctgtaaatttgcaaaatcattgatagggaatgcatggcttacagatttgttgatcatttgtgactgcccagcttctccatttttttttttttttttttttggtcccttgtctagagtcagggcagtttgaggtcagttccatggagtctttctacaaagatctttcatgtcaagcaaatacttcttttagggcactgtaggaggtgagcaggcagccgtcaaagaccttggaagagtcaataagtagggtacccgcaagtgaaaatttttctcattccacaataccagaaggccgatttcttaggaagtttcagtttcttgtatgtatcaggcactatgctaggtgctggctgtacaatgataaagtttcctccgttactgagttcacagtctatcagcctagtgggaaagaacagagaacataaggaaaatgcatgaggtgtactatatatgtttaactgtacatagtacatacaaaagctatacagcacaagtaaagcctacaggacatgcttacaggcaagctctgtctacttcttaaatcaactgtaatccattaattccagtccccatataacagctatcttctccaggaatattgaccagtacggcctacccgttttacttggtaaatgtggcaaagcagaccaccaggtcaattcagttcaattcaattctccttgacgcaaggtaacctgaaggagggtcaagttctgtccttggagttcagacagtagattccagtcctgatgctgttcataactcaatataggcttctgtctctccagaatatgagatattttttctattatcttctcctttatgttgttaattatataaattgtacttatattattttatttatttatttttcatagtggcttaaatgtccttctctgctaatgtcaacatgtgaataacatgtgaattacatttatccatcttttgtttttgttatgatcatttggtctctctttttttattttttagcatgtctcataatttctccttaatgctggactttttaaatggatacttaggtaggacctggatgatgttctcttcctctaaagaacaagaaaagttttcttttttacaggcaaagggagaaccaggcattacctcgatcctggccagagttagttttagcctttgttagggctggtctattttaacattatcttagtactggtacttacttcccaactgcgttctttagccctagtgtgggctatttctcggcctcaactacaacctgcgtgcgtttatcaaagcccttccaccttggcagagctaaactctaacttctgtttctccaacactactcaggtgctgaaatctctgcccaacttttagcttcctgggtgctgttttctgctgtgtttcttggagcctcttcctgaacatgcagcttaggagacggccaataattggaatgaattcctgtcccctaagcccaataagaattccattttatgtttgagctctctctccctcatttggaaattaccctcatgggaaaagccaggcaaacatgaattttaccttggatatttttcttctttcaaggagtataactctccaactcaattcaggactgcattgtttgctttcataggcattcaaacaggtaggggtgtgtgtgtggtggggcagggtgtgtgtgtgtgtgtgtgtgtgttccaaatactatagttgtttttatggaggggagaggtacagacatccccagcttacaatggtttgatgatttttccactttacaatggtgtgaaagagatatgcattgagtatgctccttgacttaagatttgtttactgggaggtaaccttaccataagttgaatagcatttgcattccagttatctattgccactctgaaacatcgaactgcaaaactcagcagcttaaaacaacaatttatgtttctgtgagttggctcagctggagagtttactagggaatgttcatgcaattacaatgagatggcagctgagctgtggttatctgaagcaaactgtactggacttgtaagatgtcttcttcattcttctgtctctttcttcagtgctccttgacttattttactttctactaattatctcagaatccagggcctaggcatctcacagcaagatctcaggttatttatgcttctggcattgtgactcactgctaagaggcagaaagcagtaatgtccaggtttgataagggttgcatctgtaaatggcacaggataattaccattatattttgtcagtcaaataagtcacaaggaccacccaaattccaggatagagaagcagattccacttcttcatgggagagtggcaaggtcacactacagaagagcatgtgagatgagataagattgtgtgcaattttggcagatacaatctaccatagatacaacctattcttctattgtttactttatgatttttccactttatgatggtgcaaaagtaccatgcatttggtgtgctccttgactgaagaagagtttattgcagggtagctcaactaggttaatgaaacaccataaatcttttaaacaagaagtctcaatcaaaactgactgaagaagatatgaaaaatctaaataggctgggagcggtggctcacacctgtaatccgagcactttgggaggccgaggagggtggatcacctgaggtcagaagttcaataccagcctggccaacatggtgaaaccccgtctctactacaaaaattcgctgggcctaatggcatgcgcctgtaatgccaactacatgggaagctgggaaaggagaattgcttgcacccgggaggcagaagttgcagtgagcagaggtgacgccattgcactccagcctgggcaacaagagcaaaactgtctcaaaaaaaaaaaagaaagaaagaaagaaagaaaaatctcaatagtctcgttaccatcaagtcaagtcaagttctgccaagcatttaatatacaaagtcaagtcaaatgcttccagatcagaaaatgaggaaacattatcaaacttactatatttgtgaatctattcgtgctactgtcatgaaatatctgagactgggtaatttataaagaacagaaatttatttttcatagttctggagctgggaagtcgaagatcaaggcgctagcaggttccgagtctggtcaggtcagctcttcatttccaagatggtgccttgttgctgtaatgttcccctctggaggaatattgtgtcttcagatggcaaaagagatggaaggggtgaaaagggttgtattccctctgtcaagaccttttataagaaacctaatcccattgatgagagtgtaactgtcatgacttaatcaccccctcaagcccacgcctctcaatactgctgcattgggattaggtttcaacgtgaattttgaagggaacaaaaacagtagcataaccttaattccaaaactatacagagatgcaacaggagatgaaaattgcagcctaactttattcagggatattgaggcaaacatcctaatgaagtatcagcaaaacaattgtgttggctaaggttctttgagaagcaaaaagtgggcttagatgtacaagagacttgtagagaaggataaatgggaagaagtaggagtaggcaggtaagacttcaggttttaatttaactcttacaccagcgaaaggagagagggaaggatggaggaataggagtacctcggattgcagggcagactcaagaaaattttggccaagtcaatgagggggtccttaagtcagtcacagtttagatgaattttatgtcccataagaatgggtcagtatcataatctttaccacatgcagtcattggttgggaggaggctgggggaagcatgcttttgacccagatgtagtgatgatccagaagggtggcaagtggagctgtcagttaatgatgctccctgaaacaagagatgttcatgatctattttccttgccactacacaaatctagcaatatattctaagatgtttaaatgtattatcagtgtataaagataatatgcatgttggatatagctcagaaaagtaatattattttaatattcaaaattgatgtacacaatttaatacattaagaaattagaggaaaaaaatggtgagatgcaatggctcacacctgtaatcctagtgctttgggaggctgaagtgggtggatcacctgatcccaggagtttgaggccagcctaagcaatatagtgaaacctcatctgtgcaacaaattaaaaaaaattaagaagaaaataatttaaaaaacaattagatgggcatggtggtgcatgcctgtagtctcagctacccaggaggatgaagtgggaggattgcttgagccccagggattgaggctgcagtgagttatgattgaaccagtagactccagcctgggcaacagagtgagaccctagctcaaaaaaaaaaaagagaggaagaaaaaaaattaaagcagaaaatcatgtgatggagaggggaaaaaggaaaaaacctggcaggcaggcagttagggtgggtcctcagttgaattttttcaaacaaaagaacagcctgaaaactcaagctgcaggcacagataagggaacttgaacaagggggttgcttgagacatgcctacagccacacagataagaaacactacacaggtgacttgtccagacatgcctgcaatggaaaattctgtcccctaacatgtgcacaccaaggggaacaaaacaatacggagtaactcaacctaatggcctgcatgtgcattaggaggacagggtggagctaccagaaatccacaccttatgcaaatgagatgtctagccctcatcggttttttataaaagcttttgcattcaattgtaaaaatggcaacccttttccaggccctttctccacagcagagcgctttcttcttttgtttattaaacttttgctccaacctcaccctttgtgtccacactccttaattctcttggtcatgagacaaagaactccaggcgaaacctcataacgagagactgctacattgtggtgtattggctaggctgtaacatgatctgcctaatcatgttgaaacatctaatcattatgaaaacttaagaaaaaaataggaatataaagacttttcttaacttggtaaaagatctataagaaaccaaactttgggaggctgaggtgggggttcacttgaagccaggagttcaagaccagcatggtcaacatggtgaaacctcatctctgctaacaatacaaaagaaatttagctgggcatgattgtacatgcctgtaatctcagctactcaggaggctgaggctggagaaacacttgaaccaaggagacagaggttgcagtgagcagagattgtgccactatactctagcctgggtgagggagtgataccctgtctcagaaaaaaaaaaaaagaaagaaaccaagagtaaatataaaaaacaatactgaaataatataactagtccctataagatcaaaagaatacccactatccatccccactcctatccaatattgtaccacaggaaaaaaaaaaaaaaaggtcaggcacagtggctcatccctgtaatcccagcactttgggaagccaaagtgggaggattgcttaagcccagcagtttgaggttaacctgggcaacatagtgaaaccttatatctacaaaaaattaaaaatttaactagacatggtagtccatgcctgtagtcccagctactcagtgcaggtagggctgaggtgggaggatggcttgagcctgggaagttgaggctgcagtgcactatgatcatatcactgcatttctggctgggtgaaagaatgagaccctgtctcaaaaaaataaagacttaaaaggcatagtggcctggcacagtggctcacacctgtaatcacagcactttgggaggctgaggtgggcggatcacgaatcaggagatggagaccatcctggctaacacggtaaaaccccatttctactaaaaaatacaaaaaattagctaggcatggtggtgggcgcctgtagtcacagctactcaggaggctgaggcaggagaatggcgtgaacttgggaggcggagcttgcagtgagctgagattgtgcccctgcactctagcctgggcaatagagcaatactccatctcaaaaaaaaaaaaaaaaaaaaggcatagtaatagaagagattaaaatgtcatgaattacaaatgatactattgcttatttggaaaatccaaaactatttacaggtaaattatgtataaagaaaagcaaaaggccaagaggagataagacaccccagacaagataagataagataagacaagataagataagataaggtaaggtaagatgagataaggtgagaatgggataagataaggagaatatcaagatagaaaaacacatcctactagatacggctgtagtaaaatgtaatagtgtagattggtacagaaacaggcaccaaggaaacataataaagcactcaagaatgaacccatgtgtatgaaattttatttttgatagagcaagcattgctgtaacatgcattaattagaaaattacctcgaggaggacccttaaaagggagatgccagcattatataaccaaaagtttttctgacagttttctgtaacatgtaccaacattctctttctgtcccctgtcttctgacctgaagaaatgtgttccttctttaaggaaaaaggatgatgaatggatggttttaatgtccctttatgacttttcaaggatttctttgacaatacacagatttcatacctaattgtccaccaactacaaacaaaattaattttagataaattcatagtctgaagttaattctggaaagcccatattatgtttgacaatcttaaaaacgtttctctttctatttgttgccaatttcaccccacaaatcttatctttgaattccaattaaatatggataatataaattaccattgtagaatccagtgtatgtagggttacgggtgtagtcagataactgaatgttctccttaccacctccagataaattctcttcccctctgtgattcactgatcataaatcggtttactgtgaggaaaaaaagaaatggataggttgacgttgtccccaggtggtaggtaggatgaactgcctatgaaacaatgcaatccactccaggcagtacacatttcaaaatgcttctccctatggggtttgacaggtacagacaattgttgttccaaatgccatgtgctttagcaaatcattctgcctattgtgagtagtcacctttcaagccacgagttaccatttgagttcttttgcctcaaactgatggagaaaatgaaaacagaatgtcacccatgcagttccagtctgttgtattcaagaatttgatatattcagacctatgcctggctttcagtaccatgagctaatgacatttcttggcttgactataaagttctcttgacaatactgtgtggatgagattttaccgtttgtgctaaagtcaacaacaaaagcaggacctggtaatgccaacaactatctaaagaaggtcgatcagaaaaacgacaaggtggcctattccaaagcaactctgcattcttataattttgttaatggaccttctttttaataggtttctgcaaataaagtttgaggttataaaatgtgagcctactagaatgacacaaagttgaatttcaaatgttgtttttttggatgttctaaaaatacattgtaaatggatttctgctaaggatggactactgtcttcaaagctagagaagataaattgtggaatcaagccagctgctcagagaacatagagaggtttggggtctctggttatgcatactaagtgcaaagctatcacgtggcccagaggcgggcaggcctgcttgactttaataatcagtgcctgcagtaggtagcctgtccttgggaaatgggtgtctatagctttgggcccatgtgtagcctctggttggctaccatgcccactccatttttatgctcattttcccagttccagagagatcaatgctgaggctggctaatattaattggctgtgtcattttgtccacttgactgttcagtgtgacttctgtggtgggatgctctctggtggccattaacatgtgatacaaaaactttgtgtacaaaactttttggatcacatgttaatgttttcattgctgtgcatctccattttgtccatccacctgcctctaccccagccctccttgtgtctaatggtctggtccttttctttacaggcctttgacccgctagccatgctgttcgtctcagcctagggatatctatataatcttaccttgggttccttctccttctatgccagaaacaccacttgcatcctgctcattaggaaactttttttcccctccccattatcttttttttctttttgagatggagtcttgctctgtcgcccaggctggagcgcagtggtgcaatcttggctcactgaaacctctgcctcctgggttcaagcgattctcctgtctcagcctcctgagtagctgtgattacaggcacgtgccaccgtgtctgctaatttttgtatttttagtagagacagcgtttcaccatgttggccagggtggtctcgaactcctgacctcagatgatttgcccacctcggcctaccaaagtgctgggattacaggcctgagccactgtagccagcccccactatcttctaaggccacctctgaatatgtcagcaatggaaccaccaggtattttcatcatatacccacaaactgagccaacccatatataaaccaaactcaagttgttcctcgttcatctggttgtgtggcatctctccccatagggccacgggtgtgagctggggaagggggattaatgcaaccatggatggtgacttggggacctctgggctgcctgatcctgcagcttacctgtgctctctggccctgttcaggcttaacttgggatgtatcattttcatctttagtaagatgcagtaacggtaactattcctcaatgatgtgattctctgcagatgacatggccttccttcagaatccaggggtctgcatagtatttttcctgtttggactttctataaatgacacactacatcttttctcaccgaggacccctccgacatcaaaacgtcagctggatgttatggccaatgaatcaaggctgctcattccatgggctggacctgctgcagagtccttccttgggtgttatggccaatgaatcaaggctgctcattccatgggctggacctgcgcagagtccttccttgggtgttatggccaatgaatcaaggctgctcattccatgggctggacctgctgcagagtccttccttggatgttatggccaatgaatcaaggctgctcattccatgggctggacctgctgcagagtccttccttggatgttatggccaatgaatcaaggctgctcattccatgggctggacctgctgcagagtccttccttggatgttatggccaatgaatcaaggctgctcattccatgggctggacctgctgcagagcccttccttggatgttatggccaatgaatcaaggctgctcattccatgggctggacctgctgcagagtccttccttggacgttatggccaatgaatcaaggctgctcattccatgggctggacctgtgcagagcccttccttgctctggactcacaaagcaggcagccctctgtgttagctggtgtatggacaagagcagcattccataaaccaagaaggttccaaagaggtttaacaggctgtgtgctgttctttggggaaagggatgcaagatgcagcaattgtttttcactttggagggaatagccaggcatttccctaacctctgacccctaaatactttataaagcagcaggtccctgacttttcacagggtttatcctccaccttctgctatgcatgtttcttaccaaggccttcagtgtcctggccatctcttcctggtcctgctcaatcagtatgatggcattgatatggggaatggatatgatattctatgggaggtctagatctccttggattccaccacagctgagagtggggaattaacagaagcctggagcaaaactgtaaatgaatgttcctgtccattccacgtgaatgcaaactgtttctgataagaatagaagctgggcgcagttgctcatgcctgtaatcccagcactttgggaggccgaggtgggcgaatcacctgaggtcagtggttcaagaccagtctggctaacatggcaaaactcgtctctactaaaaatacaaaaattagccaggcatggtggcacacgcctgtagtcccagctacttgggaggctgaggcaggagaatcacttgaacctgggaggtagaggttgcactgagccatggttacaccactgcactccagcttgggtgactaaatgagactccatctcaaaaaaaaaccaaaaaacacacacacacaaacccacatttgccaaatcaatggcagcacgctcttactggagacattattaatctgttctagcaatgccactttcagcactgcagccaaaataggactactacttggttgagtgtgcatcattagtctacagtcattttcttagatccagccagtttctgcaaggctcaggccagtggtgaattgaacagagataggacagagctcaccattcctgcattcctttggtttttaatgatggtatttatgtttgccaccccctggtatgtgatattgtttgtgatttactatcttgaccatggagggggcaagattcagacttctacttgaccttttctactgtgatagctcttacctcgtaagctaatgtcccaaggtggggatactcctgctgtcaagtacattcatttcaactctacacttgccgactagaaaaatgatgagtggacttgtctatgcacccagtgggcccactctgaattacactttacccagaaccctgtttgtttctttgtccccaaatcccccattctaacagaggggccatagtgttgcttcagatctctgggtactataatcagttgagagcctgtgttcaaaagtcccccaaaaggtctagagattccacttaccccagtgaacagttgctcaggtaaacagctacagatctgtctggagaaaagtcttgggaatcattacaggaattattgctgcagcattgcagagttctttctcttagggctccaactacctcttgagccaatagcttctagatctgaaaattgagtaaagtctgagaactgagcaaggtatgaaaactttttattgggtgatcaccattagttttctgctcctccattcttgccttttttcttgttgtagaaattaagaaggccccttgctggttgactgtctatttttcccctaaaggagttaggttctattaaccatcttcacaactttctgtaggccaagactctttggctgctcttccaagcttgctggtcactatggtcatcgtgactccctggcttctaatggttaagtgacaccccatcctccaccatcagtccaggcctgcagaccttatcttactagctgttttctctgatcactcctggttccaactgtgtctgttcaggtcttctaagaagtagaagccaagacaggattatatgtacaaaagatttattggatgaaatgtctgtgatatggtttggctgtgtccccacccaaatctcatcttgaattcccatgtgttgtgggagggacccagtgggagatgattgaatcatgggggcaggtctttcctgtactgttcttgtgacagtgaataagtctcatgagatctaatggtcttaaaaatgagagtttacctgcacaagctctctttttgcctgctgccatccatgtaagacatgacttgcccctccttgccttccaccatgattgtgaagccagccacgtggaactgtaagtacactcgaccactatcttttgtaaattgcccagtcttgggtatgcctttatcagcagcatgcaaacagactaatacagtctgtgacagataaaagaagctggagaagaagtaggcaggacaagccttcagatcacagtgtaagttttatatctgtaaaaggagaggaaaaaggaagaattgatctggaagagtctgagcttacagagcttctctaagaaaagtcttggctaggactatggggtgtcctgaacaaaagttacccattagaggaatctttcaacaggcaaatggtcctatgccagtgtacatgcccggctacgtcattgggtgggaacagcctagtggtataattggttcagaaagaatgcaggggtatgacagcttgagactgtcaatcagctgtattccatttctcaaagggagctctgaacctccatagtcatctgtttggtcatcagggccacacactaattattattaaagaagaatatttccgtaaatgcagcagaatctgcaaagattcctctgagcctccaatgaggagaaaatattacattttgagacagaacagtgtatgtgttcattctcatttttcagactgtgccttggctctctctgcatttgagaaaagaaactgattacatatccagccatcctttgatggcctcatcagctacacgaaggcccaattaaagttttaaaattattgttttcctttaatgttattaatatggtttggctccgtgtccccagctaaatctcatgtcaaattgcaatcctcatgtgttggaggaggggcatggtaggtggccattgaatcatgggggcggacttcccccttgctgttcttgtggtaacaatgagttctcagaagatctgcttgtttaaaagtgtgtagcatttccctcttcatgctctgtctctcctgctgctgcgtgaagaaatatttgcttccccttcccctttccactctgattgtaagtttcctgaggcctccccaggcaccttcctgtacttcctgcagaactgttagtcaattaaacctcttttctttataaatttatactaggtaatttatgtaattacccagtctcaggtagtgcttcatagcaatgtgagaagggactaacacagtcatttactcaaagtaactttccttgatcttcttctttactttgtttccttatcctaccttacttttcttctacacactcattactatctatgttattccgtatttatgtgtttgtttactttataattttctgtgtcctccattagaatgtaagtttcatgccagtagaagcctcttctgtcttgttcactggagtatcttcggtgttcagttcatactgcctggcacagagtatgtgcataacttgtgattattttgaaagagtagataaaatctgtggtaggccagcctcccccatacatacctaaaaccagtcctcagagtggaggatggggatagagaactaataacattatagacacttcacaatcatcctacaaataaagcattatcacccccattttgcagataaggaaactgtggtttatagaagttaagtaatattgacaaggtcatttggctagtaaattacaaagctgggctctgaatgcagttacacactatatgtcctcaaccactatagagtattaatagcaaaaaaaaaaaaaaactctctagcttgtaaatagatgaggttctgggaaacactggactagttcaatttccaaacttcttgaaagtaatgactctcaccatcaagctacataatcgctaaattataaaaccggcagtgtgaaaaatcctaaagcacttctgaattgtccacctaacaactgattacatcctcttctgcaaagggaaataaaactgtttgctaatcaggcctcagaaaggcttaagtgttccacaccgaatatcagaaatcgggaaaatcattaagtcattacctcgtgccaaattgaatgctacagtgtacttttctggtattttaacctgtctagcatctaatgactcagaaagcatttctcttatttaatccctctcagcgttttttttttccttactgagaagctactattaacctctcttgtaaagaaagtgagtctcttcagatatctgtcaaataattttatgtggttaacctaaagcacagctttttctaaaaaaaaaatagaaggatcgttctggaaaggagacaagaaaaagcagtccagtgggaagaacataattgtcttagcatacccgttgctggcagatgcatgaaaaagccacacaaatgagacaccagcatctaaaatcaaatgcctgtcagcttaaattgtagtctttggtaaacctgatcaattattcagccttcttacacatttaataaagctccagaacagagcccagaaaatagttgcatataaaatattctttgagaaataaattacgctttagtagaaagctacagtctttacctaagctaactcggatgatactccaagtgagctatccttcctgcttacttctgcctttttttcttcaaagggcaatcctttttgaattggcatttagcatggtagtattaggttgggggaaaaaatcgtgtttttttgccattaaaagtaatagcaataactttcgccccaacgtaatagttgcttcattcctggagagtgggatctgcttggtttaccagccagagggcttgacaaaatctgactttagtgtcttcgaatgtccttttctaacatctgcacagaaatatcttcatgggagaaaatcatctttttcattaaaaaatgtcagtgtgggagtatggggagaggaaatagggagagaaagagaaggaaagaggcgcacacatacagagacagagagagacagggagagagagagggcaagagagtactgccttggggtcactaacacccttgaagatatctcagaagattgttccaaccaagtaaaaatgtagacaaaaagtcgccgctccttcttcagacacaacggatcttgtgaagagcctggtagggcaaaaacttgagaggccattgtggtgtccaaagaaatactgacatctactggtgggttcggaagtcacaggacttttccaagggccacatttagttgcacggaattgaaagtattaaaatccagtggatacaattaggcattcctgaggatcccttctaaataaatttttaaaattaacttatgaccatcaagagcatttctgaattgtctgttctttaatccaccagatagataatcattgacttagtctctgtacctgaaatatttcacacagtccagggaagagtaaaatgaacaaaattcctttctctttatgaccccaggacctacctcatcaaaaacggtgctttgaaaaacagcaagaagaatgggaagcaatattgctcagtcagaagataaacctgtgttcacatccaggaactttcatttattggctgtgtaatttctaccaagttactttgtctcccaaatctcattctttctcagctggaaatgagaattgaggatacctgccctgaatgactctgtcagtaattagagaccacgaactcaaagtgcctgtggttcacattacacactgagtaattgccgggcgcggtggctcacacctgtaatcctagcactttgggaggccgaggcgggtggatcacctgaggtcaggtagtcgagacaagcctgaccaacatggtgaaaccccatctctactaaaaatacaaaaattagctgggtgtggtggcatgcacctgtagccccagctactcaggaggctggggcaggagaattgcttgaacctgggaagtggaggttgcagtgagctgagatggcactactgcactccagtctgggcgacaaagcaacgctccatctcaaaaaaaaaacaaaaacaaacaaataaaagaaacaaaaaaactcagtaatgggattgttgttacatttaaaggatagaaacatcctgggaccacttgcttcttgattgaccaagctgtgatttaggagagtacttctcaaactttaatttgcactcgactcactagggaatcttgttaaaatgaaggttctgtttcagctgccaagggcggggcctgagatactgcatctccagcaagctcccaagagatattgatatgctggcctgggacagcactgagtagcaagaatctgtggggtccaatccccactagaggtcaagagcattgaggtggcccctgtcaattggcttcctatgtgaggggaaatagctctcaaaatttcagttgtttcaaagtataaacaaagtgtacataagaaaattgccaggaaagatgggtttgcctaatgtagaaacactaaaaattggccgggtacagtggctcacacctgtaatcccagcactttgggaagccaaggtgggtggatcacctgaggtcaggagttcaagaccatcctggccaacatggcgaaaccccatctctattaaaaatacaaaaaaacattagctaggcatggtggcacgtgcctgtagtcccagctactcgggaagctgaggcaggagaatcgcttcaacccgggaggtggaagttgcacttagccgagattgcaccacggcactccagcctgggcaacagagcaagagactctctctccaaataaataaataaataaatactaaaaatttacttcacacccattcccaatatacagattctacttatcatgatagagaagaggtgtttgggttctgaatcagaaatgcccccataggttagacatggtaggtcacacctgcaatcctagcactttggggggctgaagtgggaagattactcgagccctggagtttgagaccaggctagtcaatgtagcgagatcctatctctacaaaaattttaaaattagccaggtgtggtggtgcatgcctgtagtcccagctactcaggaggctgaggtgggaggatcgcctgagcccagaagttcatggttgctccagctagggtgacagaatgaaatcccagtagttaatttattctgtttcagctttaggctttctgtagaatctggcaatggcaagcaattaagctaaggggatctggagtcacaggtgaatttgcatcctgactcggtaacttgccatctgccaggtcatagacaacttccttaacttttctaggcctcagatttctcagagtatattgtgaatattgtgaacaattcaatgagataatacagataacacattagcacatatactgctcaataaatgattgtattataattatttttagacaaaataatgcagctgttatttctccccagcacacttttttttgagaatagagtctctctgtgttcccaagactgatcttgaactcctgggctcaagcgatcagtctccaaattagctggtactacaggcaagagtcatcccttaatttttaagtatagcactatggctttggggaagagtgtggtagagaaagcagtggaacagcacagaatgctgttttgcaggaagtaagatgcaggtttaaatcctagttccaccaccagaatggtggaggactttgggctgggttatatagtgtctctgggcttcagggttcatctgcacaatagagagatgacaatgtaggcggtgtgaggattagaaaactctgtcaagtcctgctatgcaatcagccctcaaaattagttgctattaacaacagtgggttttttggtttatgtttttgtttttgttttgagacggagttttgctcttcttgcccaggttgtaagtgcagtagcacaatctcagctcactgtaacctctgcctcctgagttcaagttctcctgcctcagcctcccgagtagctgggattacaggcacccactactgtgcctggctagtttttagtatttttagtagaaacagggtttcaccatgttggccaggctggtcttgaactcctaacctcaggttatctacaagatccgaagtgctgagattacaggcataagccaccgtgggatattacataatacagtgggatattaatattgatatagtgttttctgacttaggttcggtagctccttagactcccctgactcctctacctagattcctgctttttttttttttcacccaggctgcagtgcagtggagtgatcttagctcactgccacctccacctctaggttcaagcgattctcctggttcagcctcctgaggagctgagggaggctgagggattgcaggcgtgtgccaccacgcccggctaatttttgtattttttagtagggacagggtttcaccaaattggccaggttcatctcaaactcccgatctcaactgatctgcctgccttagtctcccaaagtgctgggattatgggcttgagccatcatgcccagcccaaagtcatgttttaattattgtaattagttaaatgatattttaaagtatttttacacacggataattttttacggtatacaaatgttttccttacatattagcatcatcattttgtaaaacaatggtgtaaggtaggaagggtgagtgtgtttttatttatttatgtattttacaagcaaagcaacaggaactcagagaaattaagcaacttttttaagaccatggagttcgcagtgaaagatattctccctcccacagaacttatggtagagcctcatggagcagaaagtagtatttaattgggctggtgaaaatgccctgtgtttttaccactattgggtgattgagacttgagcatgattgacggaaaaagtcactgtatatcggctcagaagaaatgagttcaaggcccagcactgttacttttcaccggtgatgttgagcaaaaccatggttatctcagattcttgctttacctgtagaaggaggactttgaactagatcatagccaatagtcttctttgcttttaaactggtggtagggatggtggggttaaaagaaaaactgtagccaagttaaatttaaaataatttaattgagcaaagaacaactcatgactcagacagcatcctgagccagagtgagctcagatactccaggacagccatgtggtggaagaggatttatggacagaaaaaggaaaatgacatacagaaaatggaagtgagggacagaaacagccagattggttgttgcttggcatttgccttatctgaacatggtttgaacagttggccaatgtgattggcaaaacttggtgactggcgcaagagtaggctacagtctgtttacaattccatttaggttatggttcactatgtacagagaaacctttaggctgaacttaaaatatgtagggaggtaactttaggctaaacttgatttaacagtgttcattgccagaggaagagagatgtagaaagcaaaaagttcctcttcaaattatcccttcttgttaaagaataaatcataagtgttagaaatcatagtttcttttaaagactaactttcttcaaacctccttgcttcacactaataactcgtttttaagccctatattatgtagctgttagatataaaggaatgagtacattctatgtccttgtactttaaccaagatatctgtgctggacatgctcacaggcatgtcccagctcgcagcctataccccttccttatttaggaatgttactacttttctaagtcttttcataaacaacttccacttttcgtttgttgtacattgcttttatctatttagaaaattttaagttgttagtcaatcaggttcagtttagattgtgtggtctggctccagccaatggagacaggacacagtagcaagggcaagcactttaagggataaaaattgcttccctccttctcgccattattccatctgcgaggagcaccctttctgcagaaagtaaatttgccttgctgagaaaactttttggctgaatggtgatttttccttgcagtaccgaggaacaagcattctgtttctgaataaacattttacttataacaagagataagattaaatataagaatatattatattctctattttagataagcttgtccttgaatgttgagcatataatgtcatccaaagcatcgtccaacagcaacactattctcctgttagtaccctattctcccttttaggaaatctcccatagtgtggaagtctttctactgaaatctaagtaacagacaattcctctttctaccacaaagcagtcagggatggaggactctttccttggacttcaaatcttcagtgagtgttaggaaggtagagagatgtctgaggttcatgttcacggccacatggttgatgatttatggactagacacagcgtgctcttttgtgtgattccagctctctagcttccccagacttcttggctgttttttccaagcctggtcgtccatctgccatctcattccaataaattggaaatgcagttcaagatagctagagttggagtctgtggtattgggggaacccgcccccaatatttcaatgtaggttctttctattttccctaagtgtcggcgggtctgagaaataaagagaaagagcacaaagagaggaattttacagctgggccgctgtgggtgacatcacgtatcagtaggactgtaatgcccacctgagcctgaaagccagcaagttttattagggattttaaaaggggagggggtgcaagaacagggagtaggtcacaagatcacatgcttcaaagggcaaaaaggagaacaaagatcacatgcttctgaggaaacagggtaaggacaaaatcaggaactcctgagaagggtctatgtttagctgtgcacgtattgtcttgataaacatctcaaacaacagaaaacagggttcgagagcagagatctagtctgatcttaaatttaccagggtggggtttcccaatcccagtaagcctgagggtactgcaggagaccagggcatatatcagtccttatctcaaccgcataggacagacactcccagagtgaccgtttatagacctccccccaggaatgcaattcttttcctacggtcttaatgttaatattccttgctaggaaaagaatttagcgatatctcttctacttgcacgtccatttgtaggctctctgcaagaagaaaaatatggttttttttgcccgaccccacaggcagtcagaccatatggttgtcttcccttgttccctaaaatcgctgttattctgtttgttttcaaggtgcactgatttcacattgttcaaacacatgttttacgatcaacttttacagttaacacaatgatcacagtggacctgtggtgacgtacatcctcaacttacgaagataacaggattaagacattaaagtaagacagtcgtaagaaattttaagaatattattagggaagtgataaatgtccctgaaatcttcacaatttatgctcctctgccatggctccagccggtccttccattcgggatccctgacttcccataacatctggcttgccactaagaccctgatgaaccaagcctgcattttttttttttttttttttttttttttttttttttttttttgctaaagctactgccaaccttcttgtgatgatctgagggaatacaatttaaaccatgattcataattgtgcgaggtacatgagtcaccaagaaaggcagggtaagtaattacaaagtagttatctctctttccataattttaaattgaatatcactttattctccatttacagacagagagattacaggcagtaatcttcctttctaaagctgacagtaactcccacagtaataaatggaagcctgtttaataagaaatatttacgtgtgtgactgtgagcacagctgaaaacgtggtgttcttcgtaaatttagaactgcattttctaaatgtcgagtgtttattacctggaaaaaagaaaggaaaaacaactctacattttagacgtgttccacagcttccttgatgagacattttaaggaccattagttgtctaaatagagattgtattttagagtggagactatgaaagagtgttctctagtttaaggttatatcctttaccttgtgatagagtcaagctgtaaactcaaagcaagcatctggcactctggttattctaagtcatctttctttgctctcccttttaagcatctttcttcactttaaaaacaaacacaaataaaagcacctatcacagggagggcctcaaacgagggcttaataggtgttgctttgcttcctcttctcttttctttagtaggcagactaaattctatgactctgatctgagggttaacagtagtccatctatggattctatggacttgatgtatcttatactgtggttcctggaaattcattttatcaggagtgagtgatcttttcagaaaagctgctcaagcttgagcctgggaggtggaggttgcagtgagctgagatcatgccactgcgttccagcctggacaacagagtaagaccccatctcaaaagaaaaaaaaatctgctccagattttcagaaaggaaaataagattttacacacacacacacacacggtttttgtgtctggtaaactagcaaactgtctcatctgatttctttgccatcatttctgctttaaattagaactctagagaatgtaaatatctaattatctactggtcagcttcagagatttcaggcttcaaagaagttcagtgtggtttttgtgtgatggtttatgatatggattattcaaggaattc

SEQ ID NO: 28 - DNA, 106 bp, Homo sapiens
gagcaggaggatggcgtcctacaagccgccgggcccgtggcgctcggccggctcgaggtcggcctcgatgcactgatgcaggtgggcgtggatgtcggacccgtcg

SEQ ID NO: 29 - DNA, 106 bp, Homo sapiens
gttgtttaggttgtaggtgcgggtggacgtagttacgtagtttcggttggagttcggtcggttcgcggtgttcgggtcgtcgaatattttgcggtaggaggacgag

(19) United States
(12) Patent Application Publication
(10) Pub. No.: US 2012/0164638 A1
(43) Pub. Date: Jun. 28, 2012

(54) Digital Quantification of DNA Methylation

(75) Inventors: Bert Vogelstein, Baltimore, MD (US); Kenneth W. Kinzler, Baltimore, MD (US); Meng Li, Baltimore, MD (US); Luis Diaz, Ellicott City, MD (US); Nickolas Papadopoulos, Towson, MD (US); Sanford Markowitz, Pepper Pike, OH (US)

(73) Assignees: CASE WESTERN RESERVE UNIVERSITY, Cleveland, OH (US); THE JOHNS HOPKINS UNIVERSITY, Baltimore, MD (US)

(21) Appl. No.: 13/263,020

(22) PCT Filed: Apr. 6, 2010
(86) PCT No.: PCT/US2010/030084

§ 371 Date: Dec. 7, 2011

Related U.S. Application Data
(60) Provisional application No. 61/166,970, filed on Apr. 6, 2009.

(51) Int. Cl. C12Q 1/68; C07H 21/04

(52) U.S. Cl. 435/6.11; 536/24.33

(57) ABSTRACT

Abnormal DNA methylation can be used as a biomarker in cancer patients. For such purposes, it is important to determine precisely the fraction of methylated molecules in an analyzed sample. A technology we term Methyl-BEAMing achieves this goal. Individual bisulfite-treated DNA molecules can be PCR-amplified within aqueous nanocompartments containing beads, resulting in a population of beads each containing thousands of copies of the template molecule. After hybridization with probes specific for methylated sequences, the beads can be analyzed by flow cytometry. This approach enables detection and enumeration of one methylated molecule in a population of ˜5000 unmethylated molecules. Methyl-BEAMing provides digital quantification of rare methylation events and is generally applicable to the assessment of methylated genes in clinical samples.

This invention was made using funding from the National Institutes of Health. The U.S. government retains certain rights in the invention according to the terms of CA 43460, CA 57345, CA 62924, and CA 121113.

### TECHNICAL FIELD OF THE INVENTION

This invention is related to the area of nucleic acid analysis. In particular, it relates to analysis of methylated nucleic acids.

### BACKGROUND OF THE INVENTION

In humans, DNA methylation is largely restricted to cytosines within 5′-CpG dinucleotides. This covalent modification of DNA functions as an important mediator of gene regulation and, together with covalent modifications of histone proteins, forms the cornerstone for the burgeoning field of epigenetics. In addition to its role in development, imprinting, and cellular differentiation, DNA methylation is altered in cancer cells. Though cancers are globally hypomethylated^{1, 2}, specific regions of genes have been shown to be hypermethylated in associated with transcription silencing^{3,4}. Reversal of the hypermethylation with pharmacological agents such as 5-azacytidine can reactivate such genes⁴. Though many regions of the cancer genome are abnormally methylated, the methylation and silencing of tumor suppressor genes are particularly important. In such cases, methylation serves as a heritable mechanism to inactivate a tumor suppressor gene, with the end result similar to that resulting from mutational inactivation of the gene.

In addition to its implications for gene regulation, DNA methylation is providing a new generation of cancer biomarkers^{5, 6}. Though mutant sequences provide exquisitely specific biomarkers of this class, their utility is compromised by their heterogeneity: the same gene can be mutationally inactivated through many different mechanisms or mutated at many different positions. In contrast, DNA hypermethylation in cancers often affects identical residues in the regulatory regions of particular genes, providing significant advantages in biomarker test design. Accordingly, many studies have employed DNA methylation of specific genes for diagnostics development⁷⁻¹³. Such diagnostic tests can in principle be used for early detection of cancers, prognosis, assessing the effects of therapy or detecting residual diseases.

Many diagnostic tests based on DNA methylation have employed bisulfite to convert cytosine residues to uracils. This conversion alters the sequence of DNA, providing an opportunity to assess DNA methylation with allele-specific PCR, restriction digestion or specific hybridization probes¹⁴⁻¹⁸. Such techniques have been enormously useful for establishing the position of hypermethylated sequences in the cancer genome as well as for documenting the potential utility of such methylation for diagnostic purposes. However, in many important diagnostic scenarios, DNA from the cancer represents only a small fraction of the total DNA in the clinical sample. Such scenarios include the use of DNA from plasma/serum, urine, feces, or sputum for early diagnosis or therapeutic monitoring and the use of DNA from surgical margins or lymph nodes to monitor the extent of disease¹⁹. Currently used techniques for assessing methylation generally do not provide direct quantitative information about the fraction of DNA fragments that are methylated or are incapable of detecting small fractions of methylated fragments. The actual fraction of methylated fragments can be determined through cloning and sequencing of PCR products or next-generation sequencing technologies20, 21, but these approaches cannot easily be used in a clinical diagnostics setting.

There is a continuing need in the art for sensitive and accurate analytic methods.

### SUMMARY OF THE INVENTION

According to one embodiment of the invention a method is provided for determining fraction of molecules comprising a methylated sequence in a sample of analyte DNA molecules comprising the sequence. A sample of analyte DNA molecules is treated with a reagent which selectively modifies methylated cytosine residues or which selectively modifies unmethylated cytosine residues. Microemulsions are formed comprising the treated analyte DNA molecules. A portion of a treated analyte DNA molecule is amplified in the microemulsions in the presence of beads. The portion comprises one or more 5′-CpG methylation sites. The beads are bound to a plurality of molecules of a primer for amplifying the analyte DNA molecules. A plurality of copies of analyte DNA molecule are formed covalently attached to the plurality of molecules of the primer which are bound to beads. Nucleotide sequences at the one or more 5′-CpG methylation sites of analyte DNA molecules which are bound to beads are determined and beads that have modified 5′-CpG methylation sites and beads that have unmodified 5′-CpG methylation sites are quantified.

According to another aspect of the invention a bead is provided which is bound to a plurality of molecules of a primer for amplifying DNA molecules. The primer comprises at least 15 contiguous nucleotides selected from the group consisting of 5′-GTTGTTTAGG TTGTAGGTGN GGG-3 (SEQ ID NO: 2), 5′-CTCNTCCTCC TACCNCAAAA TATTC-3′ (SEQ ID NO: 3), and the complements thereof.

These and other embodiments which will be apparent to those of skill in the art upon reading the specification provide the art with

### BRIEF DESCRIPTION OF THE DRAWINGS

FIG. 1 Schematic of Methyl-BEAMing. “Mixtures” represent beads from aqueous nanocompartments that contained both methylated and unmethylated vimentin fragments. “Virgin” beads represent those from aqueous nanocompartments that did not contain any vimentin fragments.

FIG. 2 Representative results of Methyl-BEAMing obtained with flow cytometry. The number of beads representing methylated, unmethylated, mixture, and virgin beads are indicated in the top right corner of each box.

FIG. 3 Vimentin Methyl-BEAMing of plasma from colorectal cancer patients. (a) Receiver operating characteristic (ROC) curves based on the number of methylated vimentin fragments for the indicated classes of samples. (b) The number of methylated vimentin fragments in 2 ml plasma from normal, age and sex-matched control patients. (c) The number of methylated vimentin fragments in 2 ml plasma from colorectal cancer patients of various stages. The doted line represents 1 methylated fragment per 2 ml plasma DNA.

FIG. 4 Vimentin Methyl-BEAMing of fecal DNA from colorectal tumor patients. (a) Receiver operating characteristic (ROC) curves based on the fraction of methylated vimentin fragments for the indicated classes of samples. (b) The fraction of methylated vimentin fragments in 4 g feces from normal, age and sex-matched control patients. (c) The fraction of methylated vimentin fragments in 4 g feces from patients with colorectal adenomas (d) The fraction of methylated vimentin fragments in 4 g feces from patients with colorectal carcinomas (Duke's A or B red; Duke's C or D, blue). The dotted line represents 2% methylated fragments.

FIG. 5 (supp. FIG. 1) Correlation between the results of Methyl-BEAMing and the fraction of methylated fragments used as templates.

FIG. 6. (supp. FIG. 2) Genome equivalents of total DNA in 2 ml plasma from the indicated subgroups of patients.

FIG. 7 (supp. FIG. 3): Genome equivalents of total DNA in 4 g stool from the indicated subgroups of patients.

### DETAILED DESCRIPTION OF THE INVENTION

The inventors have developed a technology for direct quantification of DNA methylation at specific sites that is readily applicable to clinical samples, even when the fraction of methylated fragments in such samples is minute. This approach can be applied to determine the extent of methylation of marker genes in plasma and fecal DNA from cancer patients and healthy controls.

Beads are also known as microspheres or microparticles. Particle sizes can vary between about 0.1 and 10 microns in diameter. Typically beads are made of a polymeric material, such as polystyrene, although nonpolymeric materials such as silica can also be used. Other materials which can be used include styrene copolymers, methyl methacrylate, functionalized polystyrene, glass, silicon, and carboxylate. Optionally the particles are superparamagnetic, which facilitates their purification after being used in reactions.

Beads can be modified by covalent or non-covalent interactions with other materials, either to alter gross surface properties, such as hydrophobicity or hydrophilicity, or to attach molecules that impart binding specificity. Such molecules include without limitation, antibodies, ligands, members of a specific-binding protein pair, receptors, nucleic acids. Specific-binding protein pairs include avidin-biotin, streptavidin-biotin, and Factor VII-Tissue Factor. Primers can be attached via spacer moieties. The spacer moieties may be oligonucleotides.

Beads, after being prepared according to the present invention as product beads, have more than one copy of the same nucleic acid molecule bound to them. Preferably each bead is bound to at least 10, 50, 100, 500, 1000, or 10,000 molecules of the same nucleic acid sequence. In some circumstances some of the product beads are bound to more than one type of nucleic acid molecule. These product beads are generally less useful in the analysis of ratios of methylated sequences in a population of sequences. Such product beads can be readily discriminated and so do not distort the analysis.

A population of product beads will often comprise two or more types of nucleic acids. Such a population is heterogeneous with respect to the nucleic acids. Desirably, a substantial proportion of the product beads comprise only one type of nucleic acid per bead. A substantial proportion can be for example, at least 1%, at least 5%, at least 10%, or at least 50%. A product bead with only one type of nucleic acid per bead is termed homogeneous. A product bead with two types of nucleic acid per bead is termed heterogeneous. Heterogeneous product beads may result from aqueous compartments which have more than two molecules of template of non-identical sequence. This could result, for example from incomplete modification of methylated cytosine residues. A population of product beads can be heterogeneous as a population but contain individual product beads that are homogeneous.

Individual product beads preferably comprise more than one copy of template analyte molecule. Each bead may comprise at least 10, at least 50, at least 100, at least 500, at least 1000, or at least 10,000 copies of template analyte. If the bead is homogeneous, each of those copies will be identical.

Populations of product beads can be maintained in a liquid suspension. Alternatively they can be sedimented and dried or frozen. The latter alternatives may be beneficial for storage stability.

Analysis of populations of product beads can be used for distinguishing between methylated and non-methylated or hypomethylated templates. Polynucleotides can be distinguished which differ by as little as a single methylated residue, although differences in multiple residues may by better for detection sensitivity. Increased sensitivity obtained using multiple residues may decrease specificity, however.

One very convenient way for distinguishing between the products of methylated and non-methylated CpG islands is by differentially labeling probes to the expected products with fluorescent dyes. Such labeling can be accomplished by hybridization of a fluorescently labeled oligonucleotide probe to one species of polynucleotide. Alternatively, a fluorescently labeled antibody can be used to specifically attach to one oligonucleotide probe that hybridizes to a particular product. Such antibody binding can be, for example, mediated by a protein or polypeptide which is attached to an oligonucleotide hybridization probe. Of course, other means of labeling polynucleotides as are known in the art can be used without limitation. Another means of labeling different polynucleotide species is by primer extension. Primers can be extended using labeled deoxyribonucleotides, such as fluorescently labeled deoxyribonucleotides.

Populations of product beads can be used as templates. Template analyte molecules on the product beads can be analyzed to assess DNA sequence variations by hybridization, primer-extension methods, mass spectroscopy, and other methods commonly used in the art. Template analyte molecules on product beads can be employed for solid phase sequencing. In one solid phase sequencing technique, product beads are arrayed by placing them on slides spotted with complementary oligonucleotides. In another solid phase sequencing technique, product beads are placed into individual wells. In still another solid phase sequencing technique product beads are incorporated into acrylamide matrices (with or without subsequent polony formation). Sequencing reactions can be performed with any solid phase sequencing method, such as those using unlabeled nucleotide precursors (e.g., pyrosequencing, as described in Ronaghi et al., Anal. Biochem. 267: 65-71, 1999) or labeled nucleotides (e.g., photocleavable reagents described by Mitra et al., Anal. Biochem. 320:55-65, 2003). Product beads can thus be used for and facilitate massively parallel sequencing. Product beads can also be used in sequencing employing Type IIS restriction endonucleases. Product beads can also be used to provide templates for conventional dideoxynucleotide sequencing. To obtain useful data upon sequence analysis, a homogeneous template population is desirable. To provide a homogenous template population, product beads can be diluted, separated, or otherwise isolated so that each sequencing reaction contains a single product bead. Alternatively, product beads can be sorted to provide populations of beads with a single species of template.

Oligonucleotide primers can be bound to beads by any means known in the art. They can be bound covalently or non-covalently. They can be bound via an intermediary, such as via a protein-protein interaction, such as an antibody-antigen interaction or a biotin-avidin interaction. Other specific binding pairs as are known in the art can be used as well. To achieve optimum amplification, primers bound to the bead may be longer than necessary in a homogeneous, liquid phase reaction. Oligonucleotide primers may be at least 12, at least 15, at least 18, at least 25, at least 35, or at least 45 nucleotides in length. The length of the oligonucleotide primers which are bound to the beads need not be identical to that of the primers that are in the liquid phase. Primers can be used in any type of amplification reaction known in the art, including without limitation, polymerase chain reaction, isothermal amplification, rolling circle amplification, self-sustaining sequence replication (3SR), nucleic acid sequence-based amplification (NASBA), transcription-mediated amplification (TMA), strand-displacement amplification (SDA), and ligase chain reaction (LCR). Primers can be designed to hybridize only to modified, only to non-modified, or to both modified and non-modified sequences. Mixtures of primer sequences can be used to capture variants.

Microemulsions are made by stirring or agitation of oil, aqueous phase, and detergent. The microemulsions form small aqueous compartments (nanocompartments) which have an average diameter of 0.5 to 50 microns. The compartments may be from 1 to 10 microns, inclusive, from 11 to 100 microns, inclusive, or about 5 microns, on average. All such compartments need not comprise a bead. Desirably, at least one in 10,000 of said aqueous compartments comprise a bead. Typically from 1/100 to 1/1 or from 1/50 to 1/1 of said aqueous compartments comprise a bead. In order to maximize the proportion of beads which are homogeneous with respect to oligonucleotide, it is desirable that on average, each aqueous compartment contains less than 1 template molecule. Aqueous compartments will also desirably contain whatever reagents and enzymes are necessary to carry out amplification. For example, for polymerase chain reaction (PCR) the compartments will desirably contain a DNA polymerase and deoxyribonucleotides. For rolling circle amplification a DNA polymerase and a generic DNA circle may be present.

Emulsions can be “broken” or disrupted by any means known in the art. One particularly simple way to break the emulsions is to add more detergent. Detergents which can be used include, but are not limited to Triton X100, Laureth 4, Nonidet.

Sample DNA for amplification and analysis according to the present invention can be genomic DNA. Samples can be derived from a single individual, for example, from a body sample such as urine, blood, serum, plasma, sputum, stool, tissue or saliva. Samples can also be derived from a population of individuals. The individuals can be humans, but can be any organism, plant or animal, eukaryotic or prokaryotic, viral or non-viral.

Any type of probe can be used for specific hybridization to the amplified polynucleotides which are bound to the beads. Fluorescently labeled probes are useful because their analysis can be automated and can achieve high throughput. Fluorescence activated cell sorting (FACS) permits both the analysis and the isolation of different populations of beads. One type of fluorescently labeled probe that can be used is a modified molecular beacon probe. These probes have stem-loop structures and an attached fluorescent moiety on the probe, typically on one end of the probe, sometimes attached through a linker. Unlike standard molecular beacon probes, modified molecular beacon probes do not have a quenching moiety. The modified molecular beacon probe can have the fluorescent moiety attached on either end of the probe, 5′ or 3′. One such probe will hybridize better to a sequence from a methylated gene than to a non-methylated gene. Another such probe will hybridize better to a sequence from a non-methylated gene than to a sequence from the methylated gene.

Microemulsions or nanocompartments are formed with beads and primers. Because BEAMing requires thermal cycling, an emulsifier which is thermostable can optionally be used. One such emulsifier is Abil® EM90 (Degussa-Goldschmidt Chemical, Hopewell, Va.). Other such emulsifiers can be used as are known in the art.

Chemical reagents can be used which selectively modify either the methylated or non-methylated form of CpG dinucleotide motifs. Alternatively, methylation-sensitive restriction endonucleases can be used to detect methylated CpG dinucleotide motifs. Such endonucleases may either impartially cleave within methylated CpG recognition sites relative to non-methylated CpG recognition sites or preferentially cleave within non-methylated relative to methylated CpG recognition sites. Examples of the former are Acc III, BstN I, and Msp I. Examples of the latter are Acc II, Ava I, BssH II, BstU I, Hpa II, and Not I.

Modified products can be detected directly, or after a further reaction which creates products which are easily distinguishable. Means which detect altered size and/or charge can be used to detect modified products, including but not limited to electrophoresis, chromatography, and mass spectrometry. Examples of such chemical reagents for selective modification include hydrazine and bisulfate ions. Hydrazine-modified DNA can be treated with piperidine to cleave it. Bisulfate ion-treated DNA can be treated with alkali.

One way to distinguish between modified and nonmodified DNA is to use oligonucleotide probes which are specific for certain products. Such probes can be hybridized directly to modified DNA or to amplification products of modified DNA. Oligonucleotide probes can be labeled using any detection system known in the art. These include but are not limited to fluorescent moieties, radioisotope labeled moieties, bioluminescent moieties, luminescent moieties, chemiluminescent moieties, enzymes, substrates, receptors, or ligands.

Test cells can be obtained from surgical samples, such as biopsies or fine needle aspirates, from paraffin embedded tissues, from a body fluid such as bone marrow, blood, serum, plasma, lymph, cerebrospinal fluid, saliva, sputum, stool, urine, or semen. Such sources are not meant to be exhaustive, but rather exemplary.

In one aspect of this embodiment, a gene can be contacted with hydrazine, which modifies cytosine residues, but not methylated cytosine residues, then the hydrazine treated gene sequence is contacted with a reagent such as piperidine, which cleaves the nucleic acid molecule at hydrazine modified cytosine residues, thereby generating a product comprising fragments. The fragments can then be used in amplification reactions and the products analyzed.

Bisulfite ions, for example, sodium bisulfite, convert non-methylated cytosine residues to bisulfite modified cytosine residues. The bisulfite ion treated gene sequence can be exposed to alkaline conditions, which convert bisulfite modified cytosine residues to uracil residues. Sodium bisulfite reacts readily with the 5,6-double bond of cytosine (but poorly with methylated cytosine) to form a sulfonated cytosine reaction intermediate that is susceptible to deamination, giving rise to a sulfonated uracil. The sulfonate group can be removed by exposure to alkaline conditions, resulting in the formation of uracil. The DNA can be amplified, for example, by PCR, and sequenced to determine whether CpG sites are methylated in the DNA of the sample. Uracil is recognized as a thymine by Taq polymerase and, upon PCR, the resultant product contains cytosine only at the position where 5-methylcytosine was present in the starting template DNA. One can compare the amount or distribution of uracil residues in the bisulfite ion treated gene sequence of the test cell with a similarly treated corresponding non-methylated gene sequence. A decrease in the amount or distribution of uracil residues in the gene from the test cell indicates methylation of cytosine residues in CpG dinucleotides in the gene of the test cell. The amount or distribution of uracil residues also can be detected by contacting the bisulfite ion treated target gene sequence, following exposure to alkaline conditions, with an oligonucleotide that selectively hybridizes to a nucleotide sequence of the target gene that either contains uracil residues or that lacks uracil residues, but not both, and detecting selective hybridization (or the absence thereof) of the oligonucleotide.

Methyl-BEAMing can quantitatively assess DNA methylation in clinical samples. In addition to its potential value as a platform for clinical diagnosis, these studies have illuminated several features with important implications for the use of DNA methylation in cancer diagnostics, regardless of the platform used. First, the correspondence between the number of methylated vimentin DNA fragments and mutant APC and PIK3CA DNA fragments in the plasma samples from cancer patients provides strong evidence that circulating methylated DNA fragments are derived from the cancer cells themselves. As DNA methylation is plastic within mammalian cells, it was possible that such circulating DNA was derived from non-neoplastic stromal or inflammatory cells adjacent to the tumor or from elsewhere in the host. As far as we know, this is the first study to provide evidence of this kind, as it requires quantitative measures of both methylation and mutation to make the argument.

Second, our results demonstrate that several methylation sites within a region of interest should be concurrently assessed to achieve the required sensitivity and specificity in a diagnostic test based on methylation. This conclusion is based on the fact that even with carefully optimized bisulfite treatments, ˜0.5% of individual cytosines are unconverted. PCR errors can further contribute to this background level³². As less than 1% of the DNA in some clinical samples, such as those from plasma, are derived from tumors, it is critical to be able to differentiate biologically methylated molecules from such technical artifacts.

Third, our results demonstrate that the fraction of methylated fragments derived from cancers is higher in fecal DNA than in plasma DNA. Moreover, the fraction of methylated DNA fragments derived from adenomas is sufficiently high in fecal DNA to be detected (Table 2). We did not attempt to detect circulating DNA in adenoma patients, as previous studies have shown that there is very little, if any, circulating DNA derived from pre-malignant lesions of the colon²². This result suggests that fecal DNA is the preferred analyte for colorectal cancer screening in methylation assays. However, the fraction of methylated vimentin molecules from normal individuals is substantially higher in feces than in plasma (mean fraction of 0.96% and 0.025% in feces and plasma, respectively). It is known that DNA methylation is tissue specific, and normal cells within the gastrointestinal tract presumably contribute more methylated DNA to the feces than to the circulation. This background limits the sensitivity and specificity of Methyl-BEAMing of fecal DNA by raising the threshold for positivity.

The ability to quantitatively measure the fraction of methylated molecules via Methyl-BEAMing provides several diagnostic opportunities. There is nearly a 2000-fold difference between the concentrations of methylated vimentin molecules in the plasma of advanced cancer patients compared to normal individuals (Table 1). This difference could easily be translated into simple tests for assessing the extent of disease following therapeutic procedures³⁰. One could also imagine a prenatal test for Down's Syndrome based on the assessment of methylation of DNA fragments of chromosome 21³³. The development of such a test would require discovery of sequences that are methylated in the fetus but not in adults. Both these applications require a quantitative, rather than qualitative, assessment of DNA methylation such as afforded by Methyl-BEAMing. Though sequencing-by-synthesis instruments, such as the one employed in this study, also provide such quantification, they are not well-suited for high-throughput clinical analyses because of relatively long process times and high cost.

One of the most important applications of Methyl-BEAMing is in early diagnosis. In this study, 59% of patients with colorectal cancer could be detected by a plasma-based Methyl-BEAMing assay. Note that this sensitivity is close to the maximal detectable with the vimentin biomarker, which has been shown to be methylated in 53-83% of colorectal cancers²⁵. The fact that 50% of presumably curable Duke's A and B stage cancers could be detected by this method was particularly encouraging.

Equally encouraging was the observation that 45% of patients with clinically significant pre-malignancies could be detected by Methyl-BEAMing of fecal DNA. Patients with these lesions would generally be treated by endoscopic excision without abdominal surgery. In screening an asymptomatic cohort of adults over age 50, the prevalence of colon cancers and of adenomas≧1 centimeter is expected to be 0.5-1.0% and 6.0%, respectively^{34, 35}. Detection of 41% of cancers and 45% of adenomas by Methyl-BEAMing of stool DNA, at 95% specificity, would translate into a positive predictive value of 40%. This value exceeds that achieved in many studies of mammography, a cancer screening test of established clinical value in reducing mortality³⁶.

How could the Methyl-BEAMing approach be improved? As it could in principle be applied to any gene that is hypermethylated in cancers, the sensitivity for early-stage tumors could easily be raised by including one or a few additional genes in Methyl-BEAMing assays. The specificities of plasma-based and fecal-based Methyl-BEAMing assays were 95% and 92%, respectively. Though specificities in this range are typical of diagnostic screening tests that are widely used³⁶, the inclusion of additional genes in a methylation panel would undoubtedly lower the aggregate specificity. One challenge is therefore the identification of sequences in addition to vimentin that are methylated in tumor DNA but very rarely methylated in any normal adult cells. We have tested several commonly used methylated biomarkers in the context of selecting a biomarker that would be useful for testing in a body fluid and found most of them to be unsuitable. Hopefully, currently ongoing genome-wide epigenetic projects will identify new, more specific methylated sequences in the future^{37, 38}. Another potential enhancement would involve the combined analysis of vimentin methylation with mutations of commonly mutated genes, such as KRAS, PIK3CA and TP53, which could in theory increase sensitivity without compromising specificity.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### Example 1

### Methods

Sample collection and DNA purification. Plasma samples were collected by study nurses of Indivumed GmbH, Hamburg, Germany, from surgery patients treated in hospitals of Indivumeds' collaborative network, in particular from patients at the Israelitic Hospital and Clinic Alten Eichen (both in Hamburg, Germany) following strictly controlled SOP criteria. IRB approval was provided by the Ethical Board of the Physicians Association of Hamburg and patient samples and data were collected only after informed, written consent was obtained. The samples used in the current study were randomly chosen from those contributing through this protocol. Shortly before surgery, 18 ml of blood was taken from a central catheter and loaded into a standard blood collection tube containing EDTA. The tube was immediately chilled to 8° C. and transported to the laboratories within 30 min for plasma preparation. The blood cells were pelleted for 15 min at 200 g in a Leucosep tube (Greiner, Frickenhausen, Germany) filled with 15 ml of Ficoll-Paque solution. After centrifugation, the supernatant (i.e., plasma) was transferred into 1.5 ml tubes, immediately frozen, and stored at −80° C. The plasma samples were thawed at room temperature for 5 min, and any remaining debris was pelleted at 16,000 g for 5 min. The supernatant was transferred to a new tube. DNA was purified from the plasma supernatant using the QIAamp MinElute Virus Vacuum Kit (Qiagen) as recommended by the manufacturer. The DNA was eluted in EB buffer (Qiagen), and stored at −20° C.

Stool samples and clinical data were collected from patients undergoing colonoscopy at Aarhus University Hospital. The study was approved by the Regional Scientific Ethics Committee, Aarhus, Denmark and by the Danish Data Protection Agency and informed, written consent was obtained in every case. Stool samples were collected on a weekday before bowel preparation for colonoscopy or operation for colorectal cancer. The patients placed the container in an insulated box containing frozen bags which had been stored in a freezer at least 24 hours. The box was collected at the patient's home within eight hours. Immediately after the stool sample arrived at Aarhus University Hospital, the container was frozen at −80° C. until it was thawed for DNA purification. Stool samples were homogenized with a stool shaker (Exact Sciences Corp, Marlborough, Mass.). After homogenization, a 4 g stool equivalent of each sample was subjected to 2 centrifugations (5 min at 2536 g and 10 min at 16,500 g). The supernatant was incubated with 20 μL RNase (0.5 mg/mL) for one hour at 37° C., followed by precipitation with 1/10 volume of sodium acetate (3 M) and an equal volume of isopropanol. Stool DNA was dissolved in 4 mL of TE buffer and prepared for electrophoretic capture by mixing with 4 mL of water, 1 mL of 10×TBE (890 mM Tris base, 890 mM boric acid, 20 mM EDTA), and 1 mL of 10% SDS. DNA samples were denatured at 95° C. for 15 mM and then cooled on ice for 15 min. Vimentin fragments were then purified using a Reversible Electrophoretic Capture Affinity Protocol (RECAP)³¹. This was accomplished by repeatedly driving the DNA back and forth through the capture device loaded with oligonucleotide probes for the Vimentin gene target (capture probe sequences were 5′-Biotin-CGCCACCCTCCGCAGCCATGTCCACCAGGTCCG-3′ (SEQ ID NO: 4) and 5′-Biotin-GGCCATCGCCACCCTCCGCAGCCATGTCCACCAGG-3′(SEQ ID NO: 5)). After completion of electrophoretic cycling, the capture device was moved to a wash plate and washed four times with 500 μL of ST buffer (150 mM NaCl, 15 mM Tris-HCl, pH 7.4). The captured human DNA was eluted by adding 100 pit of 0.1 M NaOH to the capture device. The eluate was then neutralized with 10 μL of neutralization buffer (60 mM Tris pH 9.0, 6 mM EDTA, 0.5 M HCl).

Real-Time PCR. The amount of DNA per 2 ml plasma samples was quantified using a modified version of a human LINE-1 quantitative real-time PCR assay 39. Two primer sets were designed to amplify differently sized regions within the most abundant consensus region of the human LINE-1 family (for the 91-bp product, the primers were 5′-TGGCACATATACACCATGGAA-3′(SEQ ID NO: 6) and 5′-TGATGGTTTCCAATTTCATCC-3′(SEQ ID NO: 7); for the 69-bp product, the primers were: 5′-TGGCACATATACACCATGGAA-3′ (SEQ ID NO: 8) and 5′-TGTCCCTACAAAGGATATGAACTC-3′ (SEQ ID NO: 9)). PCR was performed in 25 reaction volumes consisting of template DNA equivalent to 254 of plasma, 0.5 U of Platinum Taq DNA Polymerase, 1×PCR buffer (see above), 6% (v/v) DMSO, 1 mM of each dNTP, 1:100,000 dilution of SYBR Green I (Invitrogen), and 0.2 μM of each primer. Amplification was carried out in an iCycler (Bio-Rad) using the following cycling conditions: 94° C. for 2 min; 3 cycles of 94° C. for 10 s, 67° C. for 15 s, 70° C. for 15 s; 3 cycles of 94° C. for 10 s, 64° C. for 15 s, 70° C. for 15 s; 3 cycles of 94° C. for 10 s, 61° C. for 15 s, 70° C. for 15 s; 35 cycles of 94° C. for 10 s, 58° C. for 15 s, 70° C. for 15 s. Various dilutions of normal human lymphocyte DNA were incorporated into each plate to serve as standards (0.006 to 1.56 ng). The threshold cycle number was determined using Bio-Rad analysis software. Each quantification for each LINE-1 product was performed in duplicate and the average value was used to determine the number of genome equivalents in the sample by comparison to the standards.

The amount of DNA per 4 g stool samples was quantified by real-time PCR assay using three sets of Vimentin specific primers (for the 81-bp product, the primers were 5′-GGGTGGACGTAGTCACGTAG-3 (SEQ ID NO: 10) and 5′-CCTCCTACCGCAGGATGTT-3′ (SEQ ID NO: 11); for the 91-bp product, the primers were 5′-CTGTAGGTGCGGGTGGAC-3′ (SEQ ID NO: 12) and 5′-CCTCCTACCGCAGGATGTT-3′ (SEQ ID NO: 13); for the 100-bp product, the primers were 5′-CCTCCTACCGCAGGATGTT-3′ (SEQ ID NO: 14) and 5′-CTGCCCAGGCTGTAGGTG-3′ (SEQ ID NO: 15)). PCR was performed in 25 μL reaction volumes consisting of template DNA equivalent to 0.016 g stool, 0.5 U of Platinum Taq DNA Polymerase, 1×PCR buffer, 6% (v/v) DMSO, 1 mM of each dNTP, 1:100,000 dilution of SYBR Green I (Invitrogen), and 0.2 μM of each primer. Amplification was carried out in an iCycler (Bio-Rad) using the following cycling conditions: 94° C. for 2 min; 3 cycles of 94° C. for 10 s, 67° C. for 15 s, 70° C. for 15 s; 3 cycles of 94° C. for 10 s, 64° C. for 15 s, 70° C. for 15 s; 3 cycles of 94° C. for 10 s, 61° C. for 15 s, 70° C. for 15 s; 35 cycles of 94° C. for 10 s, 59° C. for 15 s, 70° C. for 15 s. Various dilutions of normal human lymphocyte DNA were incorporated in each plate to serve as standards (0.39 to 25 ng). The threshold cycle number was determined using Bio-Rad analysis software. Each quantification for each Vimentin fragment was performed in duplicate and the average value was used to determine the number of genome equivalents in the sample by comparison to the standards.

Bisulfite conversion. DNA isolated from plasma and stool was treated with bisulfite using an Epitect Bisulfite Conversion kit (Qiagen), modified for isolating degraded DNA such as that found in plasma or stool. In brief, each bisulfite conversion reaction contained 18 μl purified DNA solution, 2 μl 0.5 g/ml single-stranded sonicated salmon sperm DNA (5S DNA), 85 μl bisulfite mix and 35 μl DNA protect buffer. Bisulfite conversion was carried out in a thermocycler at 99° C. for 5 min, 60° C. for 25 min, 99° C. for 5 min, 60° C. for 85 min, 99° C. for 5 min, and 60° C. for 175 min. The cleanup of bisulfited converted DNA followed the manufacturer's recommendations for formalin-fixed, paraffin-embedded (FFPE) DNA samples with the modification that MinElute Spin Columns (Qiagen) instead of Epitect spin columns were used for DNA disulfonation and purification. Bisulfite treated DNA was eluted into EB buffer (Qiagen) and stored at −20° C.

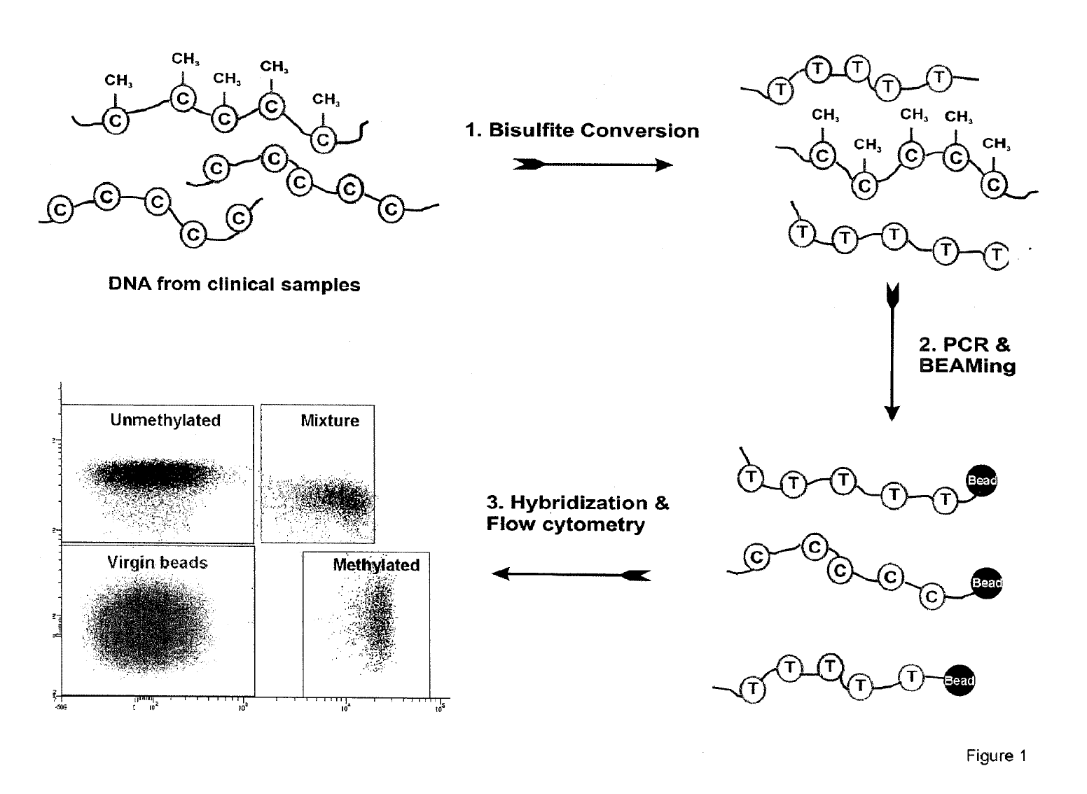

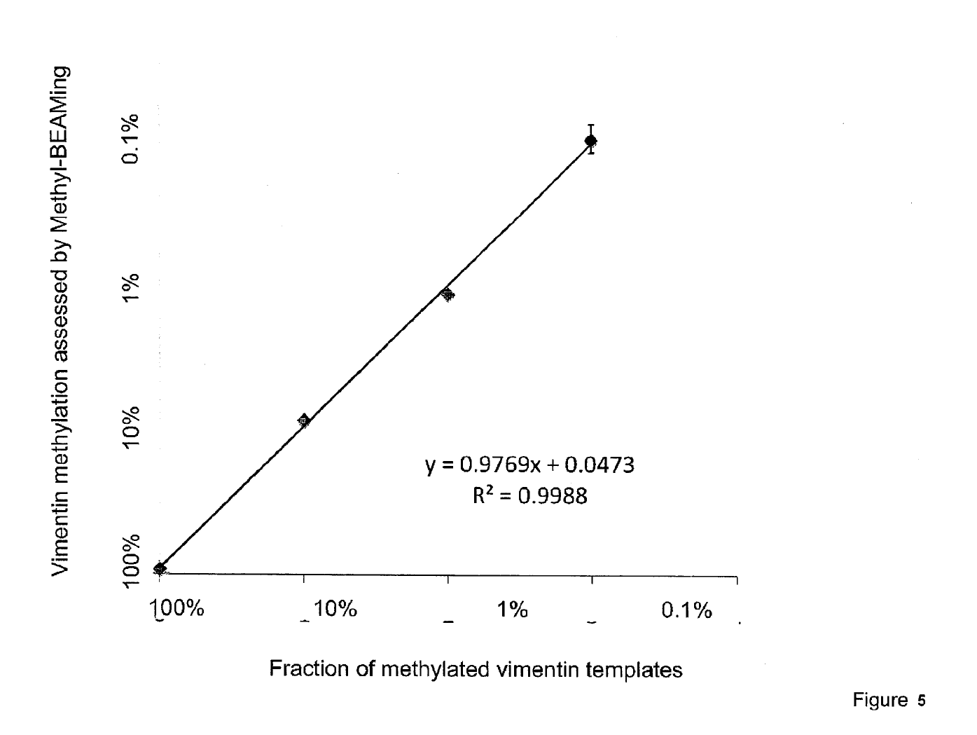

PCR amplification of bisulfite converted DNA. PCR was carried out by using a mixture of four primers that amplified both the methylated and the unmethylated Vimentin sequences (primers for methylated sequences were 5′-tcccgcgaaattaatacgacCTCGTCCTCCTACCGCAAAATATTC-3′ (SEQ ID NO: 16) and 5′-GTTGTTTAGGTTGTAGGTGCGGG-3′ (SEQ ID NO: 17); primers for unmethylated sequences were 5′-tcccgcgaaattaatacgacCTCATCCTCCTACCACAAAATATTC-3′ (SEQ ID NO: 18) and 5′-GTTGTTTAGGTTGTAGGTGTGGG-3′ (SEQ ID NO: 19)). PCR was performed in 50 μL reaction volumes consisting of 8 μl bisulfite-converted DNA, 2.5 U of AmpliTaq Gold DNA Polymerase (Applied Biosystems), 1×PCR Gold Buffer, 1 mM MgCl₂, 0.2 mM dNTP mix, 0.1 uM of each primer. Amplification was carried out in a Thermo Hybrid thermocycler using the following conditions: 95° C. for 5 min; 3 cycles of 95° C. for 45 s, 67° C. for 45 s, 72° C. for 45 s; 3 cycles of 95° C. for 45 s, 64° C. for 45 s, 72° C. for 45 s; 3 cycles of 95° C. for 45 s, 61° C. for 45 s, 72° C. for 45 s; 45 cycles of 95° C. for 45 s, 58° C. for 45 s, 72° C. for 45 s; 1 cycle of 72° C. for 10 min. Note that these primers and PCR conditions result in a preferential amplification of methylated sequences⁴⁰. This preferential amplification was confirmed using mixtures of fully methylated and fully unmethylated sequences (FIG. 5; supp, FIG. 1). Values for the fraction of methylated fragments found in the plasma and stool were accordingly adjusted (i.e., the actual fractions of methylated Vimentin fragments determined experimentally were divided by 7). This normalization had no effect on the calculated sensitivity or specificity because all samples were adjusted equivalently.

BEAMing. Emulsion PCR was performed using modifications of the procedures described by Diehl et al.²⁹. We first prepared a 150 μL PCR mixture containing 15 pg template DNA, 45 U of Platinum Taq DNA polymerase (Invitrogen), 1×PCR buffer (67 mM Tris-HCl (pH 8.8), 16.6 mM (NH4)2SO4, 10 mM β-mercaptoethanol, 1.17 mM MgCl2), 0.2 mM dNTPs, 5 mM MgCl₂, 0.05 μM universal forward primer (5′-tcccgcgaaattaatacgac-3′ (SEQ ID NO: 20)), 8 μM Vimentin specific reverse primers (5′-GTTGTTTAGGTTGTAGGTGCGGG-3′ (SEQ ID NO: 21) and 5′-GTTGTTTAGGTTGTAGGTGTGGG-3′ (SEQ ID NO: 22)) and 6×107 magnetic streptavidin beads (MyOne, Invitrogen) coated with universal forward primer (5′-dual biotin-T-Spacer18-tcccgcgaaattaatacgac-3′ (SEQ ID NO: 23)). This PCR reaction mixture was combined with 600 μL of an oil/emulsifier mix (7% ABIL WE09, 20% mineral oil, 73% Tegosoft DEC; Degussa Goldschmidt Chemical, Hopewell, Va.) and one 5 mm steel bead (Qiagen) in one well of a 96 deep-well plate (Abgene). Emulsions were prepared by shaking the plate in a TissueLyser (Qiagen) for 10 s at 15 Hz and 7 s at 17 Hz and dispensed into eight wells of a 96 well PCR plate. Temperature cycling was performed at 94° C. for 2 min; 3 cycles of 94° C. for 10 s, 68° C. for 45 s, 70° C. for 75 s; 3 cycles of 94° C. for 10 s, 65° C. for 45 s, 70° C. for 75 s; 3 cycles of 94° C. for 10 s, 62° C. for 45 s, 70° C. for 75 s; 50 cycles of 94° C. for 10 s, 59° C. for 45 s, 70° C. for 75 s.

To break the emulsions, 150 μL breaking buffer (10 mM Tris-HCl, pH 7.5, 1% Triton-X 100, 1% SDS, 100 mM NaCl, 1 mM EDTA) was added to each well and mixed in a TissueLyser at 20 Hz for 20 s. Beads were recovered by centrifuging the suspension at 3200 g for 2 min and removing the oil phase. The breaking step was repeated twice. All beads from eight PCR wells were combined and resuspended in 100 μL wash buffer (20 mM Tris-HCl, pH 8.4, 50 mM KCl). The DNA on the beads was denatured for 5 min with 0.1 M NaOH. Finally, beads were washed with 100 μL wash buffer and resuspended in 100 μL of wash buffer.

Probe hybridization. To determine the fraction of beads containing PCR products from methylated DNA templates, 0.5 uM Cy5-labeled oligonucleotide (5′-Cy5-TCGGTCGGTTCGCGGTGTTCGA-3′ (SEQ ID NO: 24), complimentary to the bisulfite converted methylated sequence), and 0.5 uM FITC labeled oligonucleotide (5′-FAM-TTGGTTGGTTTGTGGTGTTTGA-3′ (SEQ ID NO: 25), complimentary to the bisulfite converted unmethylated sequence) were hybridized to the DNA on ˜107 beads in 100 μl hybridization buffer (30 mM Tris-HCl, pH 9.5, 13.4 mM MgCl₂, 5% formamide) at 50° C. for 15 minutes. Beads were then collected with a magnet and resuspended in 200 μl 10 mM Tris, pH 7.5, 1 mM EDTA, pH 7.5.

Flow Cytometry. Beads were analyzed with a LSR II flow cytometer and data were analyzed with FACSDiva software (BD Biosciences). The flow rate was typically set at 5,000-10,000 events per second. Events were gated to exclude doublets and aggregates.

Massively parallel sequencing-by-synthesis. DNA isolated from normal lymphocytes (control) and stool was treated with bisulfite as described above. The converted DNA was PCR amplified using the same protocol as used for Methyl-BEAMing. The PCR product was purified with a QIAquick PCR purification column (Qiagen) and used to construct libraries for Solexa sequencing following Illumina's standard DNA sample preparation instructions. Briefly, (1) the PCR products were blunt-ended with T4 DNA polymerase, Klenow polymerase, and T4 polynucleotide kinase; (2) a dA was added to the 3′ end of each strand with Klenow (exo-) polymerase; (3) adapters designed for library construction were ligated to the PCR fragments; (4) ligation products were gel-purified to select for ˜180 bp fragments; and (5) PCR amplification was performed to enrich ligated fragments. The amplified library was quantified by real-time PCR and was denatured with 0.1 M NaOH to generate single-stranded DNA molecules, captured on Illumina flow cells, and amplified in situ. A Vimentin-specific oligonucleotide (5-GTGC/TGGGTGGAC/TGTAGTTAC/TGTAGT TTC/TGGTTGGA-3 (SEQ ID NO: 26), C/T indicates that the position was an equimolar mix of cytosine and thymidine) was used to prime sequencing for 36 cycles on an Illumina Genome Analyzer. Data analysis: The sequences generated were aligned to the bisulfite converted unmethylated Vimentin sequence. The following criterions were used to filter the tags for further analysis. Each tag was required (i) to pass the Illumina chastity filter; (ii) to have an average Phred sequence quality score above 20; and (iii) to have perfect match to the reference sequence at A, G, and T positions (allowing mismatched at C positions). At least 80,000 tags that met these criteria were evaluated in each case.

### Example 2

### Technical Overview of Methyl-BEAMing

The first hurdle to overcome in developing Methyl-BEAMing was bisulfite conversion. DNA in clinical samples such as plasma is already degraded to small size by circulating nucleases and is present at only a few nanograms per mililiter²². Conventional methods for bisulfite conversion further degrade DNA and are difficult to implement with samples containing only small amounts of DNA because of cumulative losses during the procedure^{23, 24}. After much optimization, we identified conditions that resulted in nearly complete (Mean+SEM: 99.4%+0.4%) conversion of dC to dU residues while preserving the majority of the initial DNA in a fashion permitting subsequent PCR amplification (see Experimental Methods for details).

The exon 1 region of the vimentin gene has been shown to be hypermethylated in colorectal cancers when compared to normal colorectal mucosae and other normal tissues^{25, 26}. Moreover, this difference is the basis for the only commercially available diagnostic test based on DNA methylation (ColoSure, LabCorp). We therefore chose this region of vimentin to assess the dynamic range and accuracy of Methyl-BEAMing. The region of vimentin queried contains 5′-CpG sites that have been shown to be methylated in cancer cells. Primers surrounding this region were designed to amplify it, whether it was methylated or unmethylated, following bisulfite conversion. The PCR products were only ˜100 bp in length so as to accommodate the small size of circulating DNA molecules^{22, 27}. These amplicons were then used for BEAMing (Beads, Emulsion, Amplification and Magnetics)^{28, 29}.

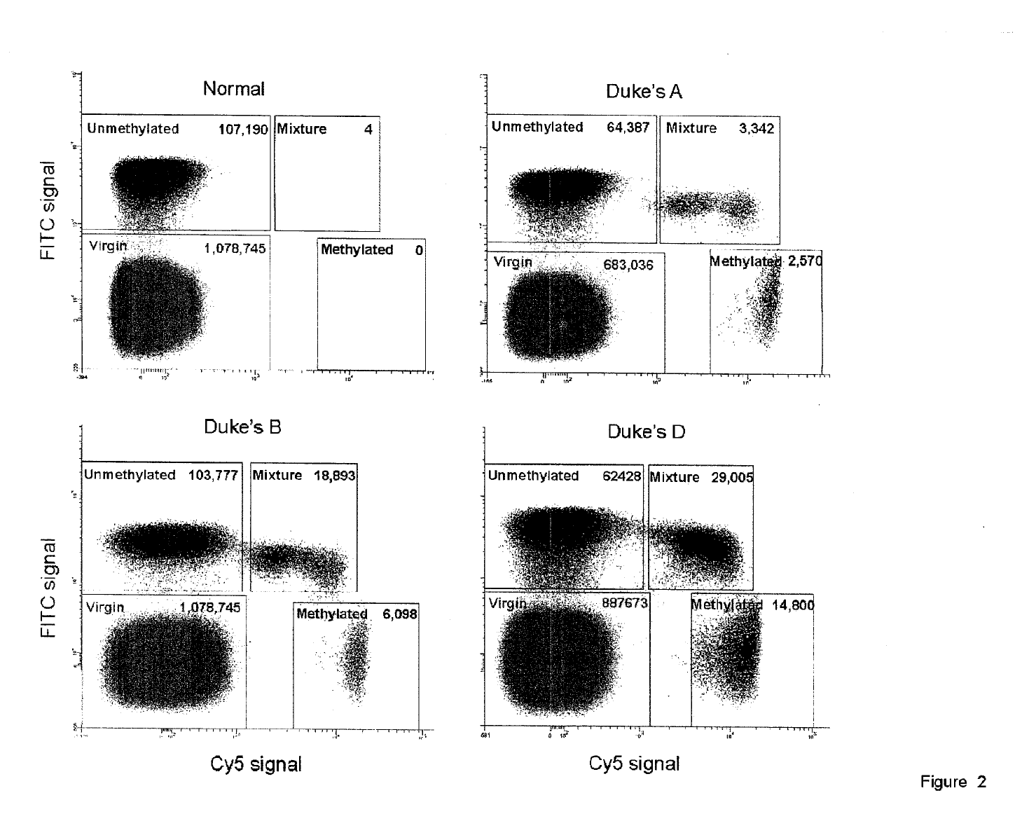

BEAMing employs aqueous nanocompartments suspended in a continuous oil phase (FIG. 1). Each aqueous nanocompartment contains DNA polymerase, cofactors, and dNTP's required for PCR. When a compartment contains a single DNA template molecule as well as a bead, the PCR product within the compartment becomes bound to the bead. Each bead thereby ends up with thousands of identical copies of the template within its nanocompartment—a process similar to that resulting from cloning an individual DNA fragment into a plasmid vector to form a bacterial colony. After PCR, the beads are collected by breaking the emulsion and incubated with fluorescent probes that specifically hybridize to either methylated or unmethylated sequences. Flow cytometry then provides an accurate read-out of the fraction of original template molecules that were methylated or unmethylated within the queried sequence (examples in FIG. 2).

As a prelude to the experiments reported below, we tested Methyl-BEAMing on mixtures of templates representing DNA from peripheral blood lymphocytes (unmethylated vimentin) and a colorectal cancer cell line (fully methylated vimentin). We found that the fraction of beads containing methylated vimentin sequences was directly proportional to the fraction of methylated input DNA when the latter represented 0.1% to 100% of the input DNA (R2=1.00, FIG. 5; supp. FIG. 1).

### Example 3

### Methylated Vimentin Gene Fragments in the Circulation Come from Cancer Cells

Some of the most important uses of cancer biomarkers involve the assessment of circulating molecules, either for early detection or disease-monitoring following therapy. To use methyl-BEAMing for such purposes, or indeed to use any test based on DNA methylation, it is assumed that the methylated molecules in the circulation emanate from cancer cells. To explicitly test this assumption, we compared the fraction of methylated vimentin molecules with the fraction of mutant molecules (APC G4189T and PIK3CA G1624A) in the plasma of two colorectal cancer patients. In these patients, the mutations were somatic and therefore exclusively present in the patient's neoplastic cells. Methyl-BEAMing was used to assess methylation and standard BEAMing was used to assess mutations³⁰. The fraction of methylated vimentin templates in the first sample was 13.6%, quite similar to the fraction of mutated APC (17.5%). In the second patient's plasma, methylated vimentin represented 3.5% of the total vimentin templates while mutated PIK3CA represented 3.0% of the PIK3CA templates.

### Example 4

### Methyl-BEAMing of Circulating DNA

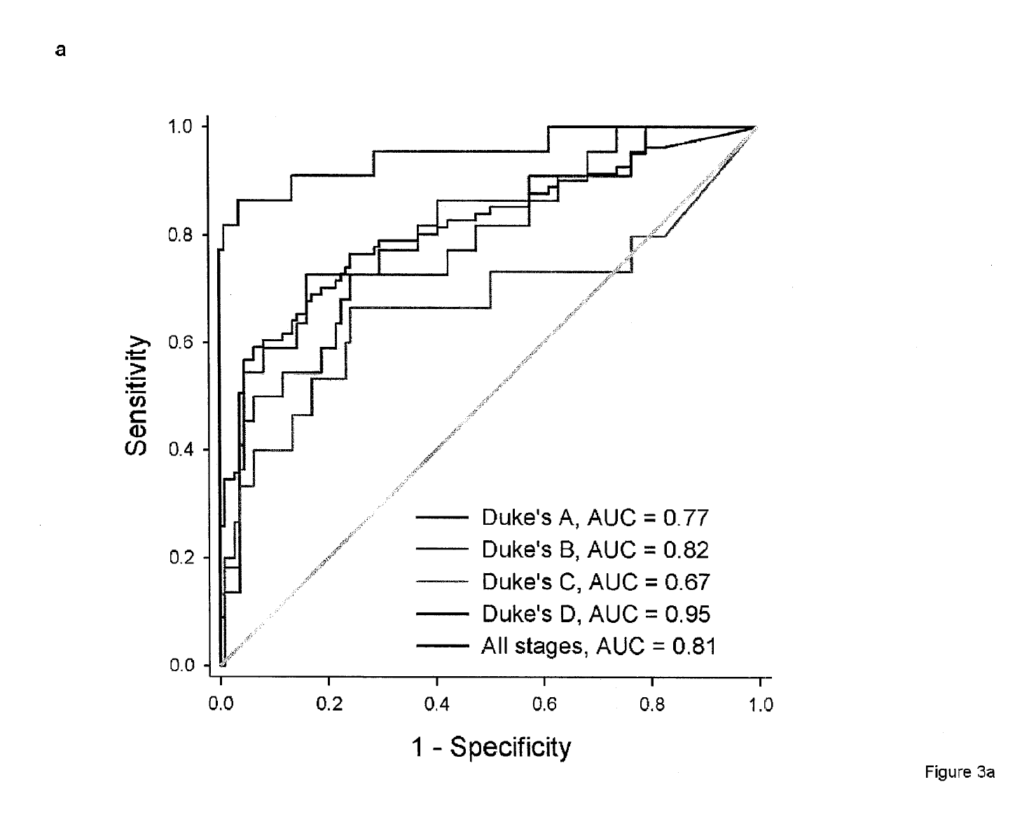

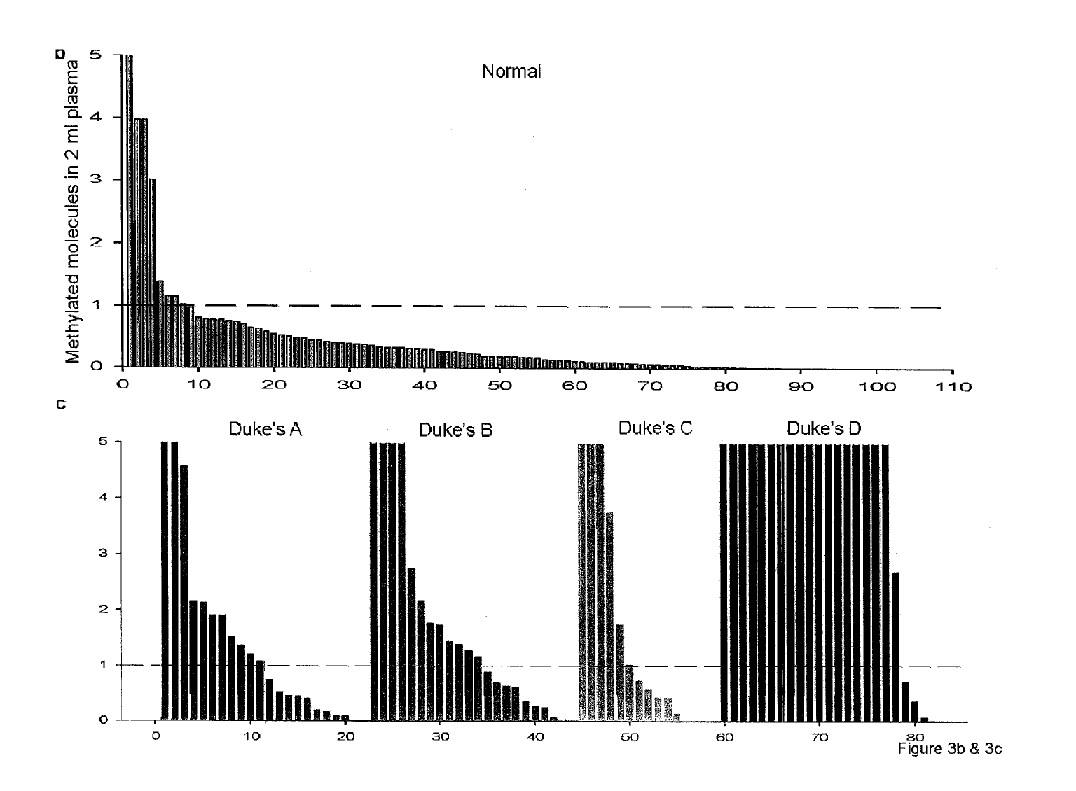

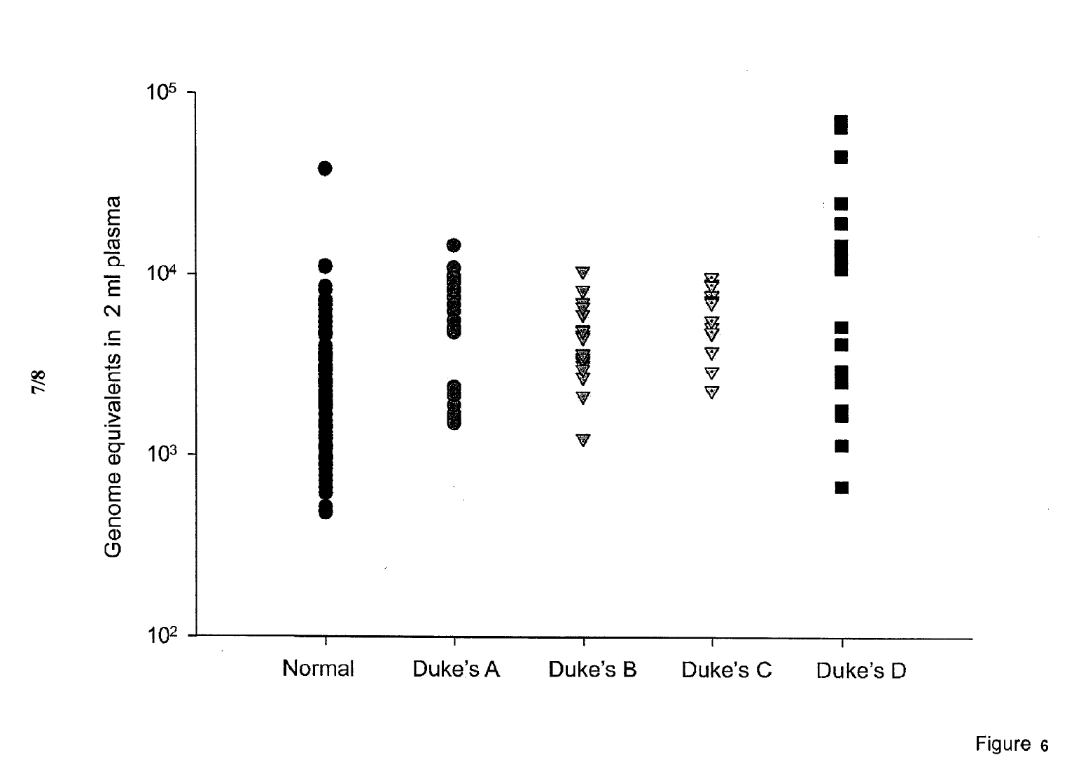

To determine the sensitivity and specificity of Methyl-BEAMing in plasma samples, we evaluated 191 samples, 81 from patients with colorectal cancer and 110 from age- and sex-matched controls. The total amount of DNA in the plasma was somewhat higher in patients with cancer than in the cancer-free controls, as noted previously 12 (FIG. 6; supp. FIG. 2). The average fraction of methylated vimentin fragments in these patients varied greatly but in a specific way. It was very low in the normal controls with a mean of 0.026% and gradually increased with cancer stage up to a mean of 4.4% in the most advanced disease (Duke's D; Table 1). The theoretical limit of detection in any digital assay is one event, i.e., in the current study, the limit of detection was one methylated vimentin fragment in the 2 ml plasma that was assayed for each patient. Using this cutoff, the sensitivity of Methyl-BEAMing was 59% in cancer patients, and the specificity was 93% (eight of the 110 normal samples contained ≧1 methylated vimentin fragment per 2 ml plasma, Table 1, FIG. 3). The Area under the Receiver Operating Characteristic Curve (AUC) of this assay in cancers was 0.81, and varied from 0.67 to 0.95 among the four cancer stages (FIG. 3a). As expected, the absolute number of methylated vimentin fragments in plasma increased with tumor stage, ranging from a mean of 1.8 in Duke's A patients with detectable circulating methylated vimentin to 1195 in Duke's D patients (Table 1 and FIG. 3c). Importantly, the sensitivity for detecting colorectal cancer was ˜50% in the patients with Duke's A and B cancers, nearly all of which were likely to be curable by conventional surgery (Table 1 and FIG. 3c).

TABLE 1
Methylated vimentin DNA fragments in plasma

| | | Total vimentin | | # of Vimentin | |
|---|---|---|---|---|---|
| | | fragments | Fraction of | methylated | # of samples with ≧1.00 |
| | | (methylated plus | methylated | fragments in | methylated fragment |
| | Number of | unmethylated) | fragments | 2 ml plasma | in 2 ml plasma (fraction |
| Sample Type | Patients | (mean ± SEM) | (mean ± SEM) | (mean ± SEM) | in parentheses) |
| Normal | 110 | 3170 ± 380 | 0.026% ± 0.013% | 0.60 ± 0.27 | 8 (7%) |
| Colorectal cancers | 81 | 8240 ± 1180 | 1.4% ± 0.40% | 334.6 ± 177.2 | 48 (59%) |
| (all stages) | | | | | |
| Duke's A | 22 | 5830 ± 781 | 0.052% ± 0.025% | 1.8 ± 0.54 | 11 (50%) |
| Duke's B | 22 | 4560 ± 458 | 0.31% ± 0.23% | 8.9 ± 5.3 | 12 (55%) |
| Duke's C | 15 | 6530 ± 632 | 0.76% ± 0.65% | 38.4 ± 31.6 | 6 (40%) |
| Duke's D | 22 | 15,500 ± 4270 | 4.4% ± 1.2% | 1195 ± 626 | 19 (86%) |

### Example 5

### Methyl-BEAMing of Fecal DNA

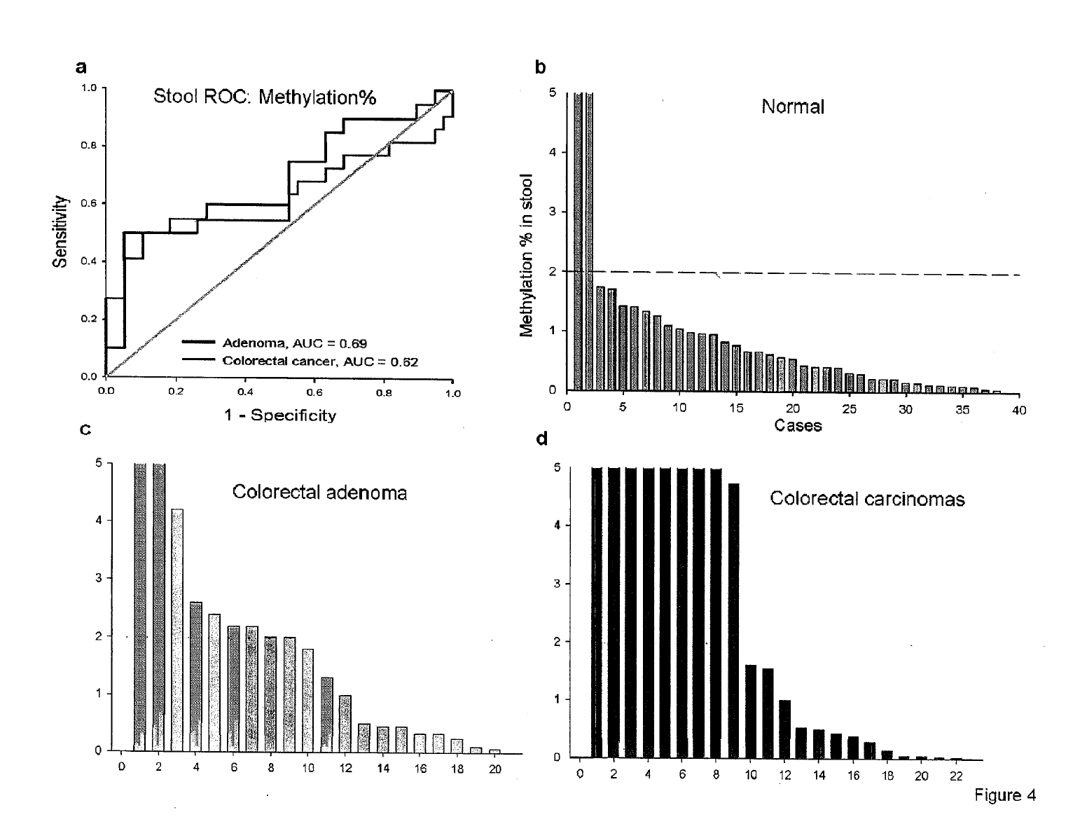

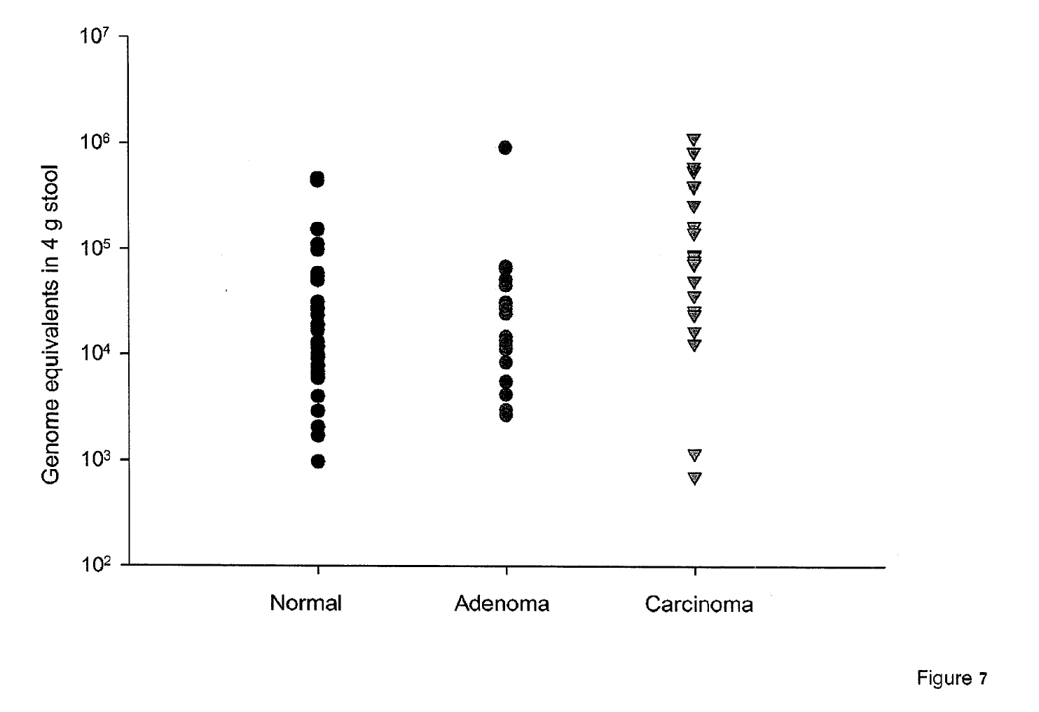

Next, we used Vimentin Methyl-BEAMing to screen Rn stool samples, including 38 from normal individuals, 20 from patients with>1 centimeter adenomas, and 22 from colorectal cancer patients of various stages. In each case, we homogenized 4 g of stool and captured vimentin fragments through electrophoresis-driven hybridization to oligonucleotides on a solid phase matrix 31. The concentration of vimentin DNA fragments in stool varied widely, as expected for samples from individuals with various diets and bowel habits (FIG. 7; supp. FIG. 3). Though there were no reproducible differences between the concentrations of total vimentin DNA fragments in the three patient groups, there was a significant difference in the fraction of methylated fragments. This fraction increased from a mean of 0.96% in normal individuals to 3.8% and 7.3% in patients with adenomas or carcinomas, respectively (Table 2). Using a threshold of 2% methylation, 45% and 41% of the patients with adenomas and carcinomas, respectively, scored positive in the Methyl-BEAMing assay, while only 5% of the samples from normal individuals did so (Table 2, FIG. 4). The corresponding areas under the ROC curves were 0.69 and 0.62 for adenomas and carcinomas (FIG. 4).

TABLE 2
Methylated vimentin DNA fragments in fecal DNA

| | | Total vimentin | | |
|---|---|---|---|---|
| | | fragments | Fraction of | # of samples with ≧2.0% |
| | | (methylated plus | methylated | methylated fragment |
| | Number of | unmethylated) | fragments | in 4 g stool (fraction |
| Sample type | Patients | (mean ± SEM) | (mean ± SEM) | in parentheses) |
| Normal | 38 | 47342.3 ± 16731.6 | 0.96% ± 0.25% | 2 (5.3%) |
| Adenoma | 20 | 69588.8 ± 45185.7 | 3.83% ± 0.86% | 9 (45%) |
| Colorectal cancer | 22 | 236182.6 ± 66576.8 | 7.30% ± 3.28% | 9 (41%) |
| (mixed stages) | | | | |

### Example 6

### Validation of Methyl-BEAMing Through Massively Parallel Sequencing

To further validate the accuracy of Methyl-BEAMing for the quantification of methylated vimetin fragments in clinical samples, we evaluated samples with low and high fractions of methylated fragments on the Illumina Genome Analyzer sequencing instrument. Following bisulfite conversion and PCR amplification (FIG. 1), the PCR products were ligated to adapters and sequenced using a standard Illumina protocol. The 36-base region sequenced encompassed the five core CpG's that were assessed with the hybridization probes employed in Methyl-BEAMing. Through the analysis of C residues not present within CpG motifs from normal lymphocyte DNA, we found that the bisulfite conversion efficiency used for Methyl-BEAMing was ˜99.4%. In this lymphocyte sample, the fraction of sequenced fragments in which four or five of the five core CpG sites were methylated was 0.015%. This fraction was determined to be 0.018% in the Methyl-BEAMing assay. Likewise, the fraction of methylated fragments in a fecal DNA sample with a high degree of methylation was similar when assessed by either sequencing or Methyl-BEAMing (11.3% vs. 10.8%, respectively).

### REFERENCES

The disclosure of each reference cited is expressly incorporated herein.
1. Feinberg, A. P. & Vogelstein, B. Hypomethylation distinguishes genes of some human cancers from their normal counterparts. Nature 301, 89-92 (1983).
2. Feinberg, A. P. & Tycko, B. The history of cancer epigenetics. Nat Rev Cancer 4, 143-153 (2004).
3. Herman, J. G. & Baylin, S. B. Gene silencing in cancer in association with promoter hypermethylation. N Engl J Med 349, 2042-2054 (2003).
4. Jones, P. A. & Baylin, S. B. The epigenomics of cancer. Cell 128, 683-692 (2007).
5. Esteller, M., Corn, P. G., Baylin, S. B. & Herman, J. G. A gene hypermethylation profile of human cancer. Cancer Res 61, 3225-3229 (2001).
6. Laird, P. W. The power and the promise of DNA methylation markers. Nat Rev Cancer 3, 253-266 (2003).
7. Grady, W. M., Rajput, A., Lutterbaugh, J. D. & Markowitz, S. D. Detection of aberrantly methylated hMLH1 promoter DNA in the serum of patients with microsatellite unstable colon cancer. Cancer Res 61, 900-902 (2001).
8. Kawakami, K. et al. Hypermethylated APC DNA in plasma and prognosis of patients with esophageal adenocarcinoma. J Natl Cancer Inst 92, 1805-1811 (2000).
9. Wong, I. H., Zhang, J., Lai, P. B., Lau, W. Y. & Lo, Y. M. Quantitative analysis of tumor-derived methylated p16INK4a sequences in plasma, serum, and blood cells of hepatocellular carcinoma patients. Clin Cancer Res 9, 1047-1052 (2003).
10. Hogue, M. O. et al. Quantitation of promoter methylation of multiple genes in urine DNA and bladder cancer detection. J Natl Cancer Inst 98, 996-1004 (2006).
11. Machida, E. O. et al. Hypermethylation of ASC/TMS1 is a sputum marker for late-stage lung cancer. Cancer Res 66, 6210-6218 (2006).
12. Fleischhacker, M. & Schmidt, B. Circulating nucleic acids (CNAs) and cancer—a survey. Biochim Biophys Acta 1775, 181-232 (2007).
13. Lofton-Day, C. et al. DNA methylation biomarkers for blood-based colorectal cancer screening. Clin Chem 54, 414-423 (2008).
14. Herman, J. G., Graff, J. R., Myohanen, S., Nelkin, B. D. & Baylin, S. B. Methylation-specific PCR: a novel PCR assay for methylation status of CpG islands. Proc Natl Acad Sci USA 93, 9821-9826 (1996).
15. Xiong, Z. & Laird, P. W. COBRA: a sensitive and quantitative DNA methylation assay. Nucleic Acids Res 25, 2532-2534 (1997).
16. Gonzalgo, M. L. & Jones, P. A. Rapid quantitation of methylation differences at specific sites using methylation-sensitive single nucleotide primer extension (Ms-SNuPE). Nucleic Acids Res 25, 2529-2531 (1997).
17. Lo, Y. M. et al. Quantitative analysis of aberrant p16 methylation using real-time quantitative methylation-specific polymerase chain reaction. Cancer Res 59, 3899-3903 (1999).
18. Eads, C. A. et al. MethyLight: a high-throughput assay to measure DNA methylation. Nucleic Acids Res 28, E32 (2000).
19. Sidransky, D. Emerging molecular markers of cancer. Nat Rev Cancer 2, 210-219 (2002).
20. Korshunova, Y. et al. Massively parallel bisulphite pyrosequencing reveals the molecular complexity of breast cancer-associated cytosine-methylation patterns obtained from tissue and serum DNA. Genome Res 18, 19-29 (2008).
21. Weisenberger, D. J. et al. DNA methylation analysis by digital bisulfate genomic sequencing and digital MethyLight. Nucleic Acids Res 36, 4689-4698 (2008).
22. Diehl, F. et al. Detection and quantification of mutations in the plasma of patients with colorectal tumors. Proc Natl Acad Sci USA 102, 16368-16373 (2005).
23. Grunau, C., Clark, S. J. & Rosenthal, A. Bisulfite genomic sequencing: systematic investigation of critical experimental parameters. Nucleic Acids Res 29, E65-65 (2001).
24. Okamoto, A. Chemical approach toward efficient DNA methylation analysis. Org Biomol Chem 7, 21-26 (2009).
25. Chen, W. D. et al. Detection in fecal DNA of colon cancer-specific methylation of the nonexpressed vimentin gene. J Natl Cancer Inst 97, 1124-1132 (2005).
26. Zou, H. et al. Highly methylated genes in colorectal neoplasia: implications for screening. Cancer Epidemiol Biomarkers Prey 16, 2686-2696 (2007).
27. Jahr, S. et al. DNA fragments in the blood plasma of cancer patients: quantitations and evidence for their origin from apoptotic and necrotic cells. Cancer Res 61, 1659-1665 (2001).
28. Dressman, D., Yan, H., Traverso, G., Kinzler, K. W. & Vogelstein, B. Transforming single DNA molecules into fluorescent magnetic particles for detection and enumeration of genetic variations. Proc Natl Acad Sci USA 100, 8817-8822 (2003).
29. Diehl, F. et al. BEAMing: single-molecule PCR on microparticles in water-in-oil emulsions. Nat Methods 3, 551-559 (2006).
30. Diehl, F. et al. Circulating mutant DNA to assess tumor dynamics. Nat Med 14, 985-990 (2008).
31. Kent Moore, J., Smith, J. A., Whitney, D. H., Durkee, K. H. & Shuber, A. P. An electrophoretic capture method for efficient recovery of rare sequences from heterogeneous DNA. Biotechniques 44, 363-374 (2008).
32. Li, M., Diehl, F., Dressman, D., Vogelstein, B. & Kinzler, K. W. BEAMing up for detection and quantification of rare sequence variants. Nat Methods 3, 95-97 (2006).
33. Lo, Y. M. Noninvasive prenatal detection of fetal chromosomal aneuploidies by maternal plasma nucleic acid analysis: a review of the current state of the art. Bjog 116, 152-157 (2009).
34. Lieberman, D. A. et al. Use of colonoscopy to screen asymptomatic adults for colorectal cancer. Veterans Affairs Cooperative Study Group 380. N Engl J Med 343, 162-168 (2000).
35. Kahi, C. J., Rex, D. K. & Imperiale, T. F. Screening, surveillance, and primary prevention for colorectal cancer: a review of the recent literature. Gastroenterology 135, 380-399 (2008).
36. Humphrey, L. L., Helfand, M., Chan, B. K. & Woolf, S. H. Breast cancer screening: a summary of the evidence for the U.S. Preventive Services Task Force. Ann Intern Med 137, 347-360 (2002).
37. Esteller, M. Cancer epigenomics: DNA methylomes and histone-modification maps. Nat Rev Genet. 8, 286-298 (2007).
38. Moving AHEAD with an international human epigenome project. Nature 454, 711-715 (2008).
39. Rago, C. et al. Serial assessment of human tumor burdens in mice by the analysis of circulating DNA. Cancer Res 67, 9364-9370 (2007).
40. Warnecke, P. M. et al. Detection and measurement of PCR bias in quantitative methylation analysis of bisulphite-treated DNA. Nucleic Acids Res 25, 4422-4426 (1997).

1. A method for determining fraction of molecules comprising a methylated sequence in a sample of analyte DNA molecules comprising the sequence, comprising:
treating a sample of analyte DNA molecules with a reagent which selectively modifies methylated cytosine residues or which selectively modifies unmethylated cytosine residues;
forming microemulsions comprising the treated analyte DNA molecules;
amplifying a portion of a treated analyte DNA molecule in the microemulsions in the presence of beads, wherein said portion comprises one or more 5′-CpG methylation sites, wherein the beads are bound to a plurality of molecules of a primer for amplifying the analyte DNA molecules, whereby a plurality of copies of analyte DNA molecule are formed covalently attached to the plurality of molecules of the primer which are bound to beads;
determining nucleotide sequences at the one or more 5′-CpG methylation sites of analyte DNA molecules which are bound to beads and quantitating beads that have modified 5′-CpG methylation sites and beads that have unmodified 5′-CpG methylation sites.

2. The method of claim 1 wherein the amplified portion of analyte DNA molecule comprises at least two 5′-CpG sites.

3. The method of claim 1 wherein the amplified portion of analyte DNA molecule comprises at least three 5′-CpG sites.

4. The method of claim 1 further comprising the step of isolating beads which are bound to modified analyte DNA molecule from beads which are bound to unmodified analyte DNA molecule.

5. The method of claim 4 wherein the step of isolating is performed using fluorescence activated cell sorting.

6. The method of claim 1 wherein prior to the step of determining, the microemulsions are broken by addition of one or more detergents.

7. The method of claim 1 wherein the step of determining is performed by hybridization to oligonucleotide probes which are differentially labeled.

8. The method of claim 1 wherein the step of determining is performed by nucleotide sequencing.

9. The method of claim 1 wherein the step of determining is performed by primer extension.

10. The method of claim 1 wherein the analyte DNA molecules are genomic DNA.

11. The method of claim 1 wherein the reagent is bisulfite ions which selectively modify unmethylated cytosine residues.

12. The method of claim 11 wherein the bisulfite ion-treated sample is exposed to alkaline conditions, whereby modified cytosine residues are converted to uracil residues.

13. The method of claim 1 wherein the sample is from a human being screened for or suspected of having a neoplasm.

14. The method of claim 1 wherein the sample is from a human being screened for or suspected of having a colon adenoma or colon cancer.

15. The method of claim 1 wherein the sample is from a patient undergoing anti-cancer therapy.

16. The method of claim 1 wherein the sample is from a patient who previously was treated to remove or ablate a cancer.

17. The method of claim 1 wherein the sample is from a tumor margin.

18. The method of claim 1 wherein the sample is from blood, serum, or plasma.

19. The method of claim 1 wherein the sample is from stool.

20. The method of claim 1 wherein the sample is from lymph nodes.

21. The method of claim 1 wherein the sample is from urine.

22. The method of claim 1 wherein the sample is from sputum.

23. The method of claim 1 wherein the sample is from a tissue.

24. The method of claim 1 wherein the amplified DNA comprises a portion of a vimentin gene.

25. The method of claim 1 wherein the amplified DNA comprises a converted sequence derived from all or a portion of
(SEQ ID NO: 1)
5′-CTCGTCCTCCT ACCGCAGGAT GTTCGGCGGC CCGGGCACCG CGAGCCGGCC
GAGCTCCAGC CGGAGCTACG TGACTACGTC CACCCGCACC TACAGCCTGG GCAGC-3′.

26. The method of claim 1 wherein the amplified DNA comprises a converted sequence derived from all or a portion of
5′-CTCGTCCTCCT ACCGCAGGAT GTTCGGCGGC CCGGGCACCG CGAGCCGGCC GAGCTCCAGC CGGAGCTACG TGACTACGTC CACCCGCACC TACAGCCTGG GCAGC-3′ (SEQ ID NO: 1), in which the converted sequence is derived by replacing each of one or more unmethylated C bases with a T base.

27. The method of claim 1 wherein the amplified DNA comprises a converted sequence derived from all or a portion of the reverse complement of SEQ ID NO:1, 5′-GAGCAGGAGGATGGCGTCCTACAAGCCGCCGGGCCCGTGGCGCTCGGCCGGCTC GAGGTCGGCCTCGATGCACTGATGCAGGTGGGCGTGGATGTCGGACCCGTCG-3′ (SEQ ID NO: 28)

28. The method of claim 1 wherein the amplified DNA comprises a converted sequence derived from all or a portion of the reverse complement of SEQ ID NO:1 in which the converted sequence is derived by replacing each of one or more unmethylated C bases with a T base.

29. The method of claim 1 wherein the amplified DNA comprises all or a portion of
(SEQ ID NO: 29)
5′-
GTTGTTTAGGTTGTAGGTGCGGGTGGACGTAGTTACGTAGTTTCGGTTGG
AGTTCGGTCGGTTCGCGGTGTTCGGGTCGTCGAATATTTTGCGGTAGGAG
GACGAG-3′.

30. The method of claim 1 wherein the primer for amplifying the analyte DNA molecules is selected from the group consisting of 5′-GTTGTTTAGG TTGTAGGTGN GGG-3 (SEQ ID NO: 2) and 5′-CTCNTCCTCC TACCNCAAAA TATTC-3′(SEQ ID NO: 3).

31. The method of claim 1 wherein the primer for amplifying the analyte DNA molecules comprises at least 15 contiguous nucleotides selected from the group consisting of 5′-GTTGTTTAGG TTGTAGGTGN GGG-3 (SEQ ID NO: 2) and 5′-CTCNTCCTCC TACCNCAAAA TATTC-3′(SEQ ID NO: 3).

32. The method of claim 1 wherein nucleotide sequences are determined by hybridization to fluorescent probes.

33. The method of claim 1 wherein the amplified portion of analyte DNA comprises between 50 and 200 nucleotides.

34. The method of claim 1 wherein the amplified portion of analyte DNA comprises between 75 and 125 nucleotides.

35. The method of claim 1 further comprising testing for the presence of one or more mutations in a tumor suppressor or oncogene in the sample.

36. A bead which is bound to a plurality of molecules of a primer for amplifying DNA molecules, wherein the primer comprises at least 15 contiguous nucleotides selected from the group consisting of 5″-GTTGTTTAGG TTGTAGGTGN GGG-3 (SEQ ID NO: 2) 5′-CTCNTCCTCC TACCNCAAAA TATTC-3′ (SEQ ID NO: 3); and the complements thereof.

37. The bead of claim 36 wherein the primer is covalently bound to the bead.

38. The bead of claim 36 wherein the primer is non covalently bound to the bead.

39. The bead of claim 36 which is superparamagnetic.
